SEQUENCE LISTING (177 sequences)

SEQ ID NO: 1 - protein, 71 aa, synthetic construct
MACKHGYPLMCPLCTALDKTSDGLFTLLFDNEWYPTDLLTVDLEDEVFYPDDPHMEWTDLPLIQDIEMEPQ

SEQ ID NO: 2 - protein, 67 aa, synthetic construct
MATTMEQETCAHSLTFEECPKCSALQYRNGFYLLKYDEEWYPEELLTDGEDDVFDPELDMEVVFELQ

SEQ ID NO: 3 - protein, 127 aa, synthetic construct
NYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEE

SEQ ID NO: 4 - protein, 219 aa, synthetic construct
MVTMAGNMICNVFAGLATEICSPKQGPLLDNELPLPLELAEFPNKDNNCWVAALSHYYTLCDVTNHVTKVTPTTSGIRYYLTAWQSILQTDLFNGYYPAAFAVETGLCHGPFPMQQHGYVRNATSHPYNFCLCSEPVPGEDYWHAVVKVDLSRTEARVDKWLCIDDDRMYLSGPPTRVKLASSYKIPTWIESLAQFCLQLHPVQHRRTLANSLRNEQCR

SEQ ID NO: 5 - protein, 67 aa, synthetic construct
MATTMEQEICAHSMTFEECPKCSALQYRNGFYLLKYDEEWYPEESLTDGEDDVFDPDLDMEVVFETQ

SEQ ID NO: 6 - protein, 71 aa, synthetic construct
MACKHGYPFLCPLCTAIDTTHDGSFTLLIDNEWYPTDLLTVDLDDDVFHPDDSVMEWTDLPLIQDVVMEPQ

SEQ ID NO: 7 - protein, 149 aa, synthetic construct
GFGHQNKAVYTAGYKICNYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEEEAMEQ

SEQ ID NO: 8 - protein, 182 aa, synthetic construct
GPEEEFGMSIIKNNTCVVTTTNGKFTGLGIYDRILILPTHADPGSEIQVNGIHTKVLDSYDLFNKEGVKLEITVLKLDRNEKFRDIRKYIPESEDDYPECNLALVANQTEPTIIKVGDVVSYGNILLSGTQTARMLKYNYPTKSGYCGGVLYKIGQILGIHVGGNGRDGFSSMLLRSYFTEQ

SEQ ID NO: 9 - protein, 229 aa, synthetic construct
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHTHCEGRAYLPHRMGKTPPMLNVPEHFRRPFNIGLYKGTIELTMTIYDDESLEAAPMIWDHFNSSKFSDFREKALMFGLIVEKKASGAWVLDSISHFK

SEQ ID NO: 10 - protein, 230 aa, synthetic construct
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 11 - protein, 512 aa, synthetic construct
MATDIDMLIDLGLDLSDSDLDEDPPEPAESRRDDLESDSSGECSSSDEDMEDPHGEDGPEPILDAARPAVRPSRPEDPGVPSTQTPRPTERQGPNDPQPAPHSVWSRLGARRPSCSPEQHGGKVARLQPPPTKAQPARGGRRGRRRGRGRGGPGAADGLSDPRRRAPRTNRNPGGPRPGAGWTDGPGAPHGEAWRGSEQPDPPGGQRTRGVRQAPPPLMTLAIAPPPADPRAPAPERKAPAADTIDATTRLVLRSISERAAVDRISESFGRSAQVMHDPFGGQPFPAANSPWAPVLAGQGGPFDAETRRVSWETLVAHGPSLYRTFAGNPRAASTAKAMRDCVLRQENFIEALASADETLAWCKMCIHHNLPLRPQDPIIGTTAAVLDNLATRLRPFLQCYLKARGLCGLDELCSRRRLADIKDIASFVFVILARLANRVERGVAEIDYATLGVGVGEKMHFYLPGACMAGLIEILDTHRQECSSRVCELTASHIVAPPYVHGKYFYCNSLF

SEQ ID NO: 12 - protein, 76 aa, synthetic construct
MACKHGYPDVCPICTAVDATPGFEYLLMADGEWYPTDLLCVDLDDDVFWPSDTSNQSQTMDWTDVPLIRDIVMEPQ

SEQ ID NO: 13 - protein, 121 aa, synthetic construct [sequence omitted]

SEQ ID NO: 14 - protein, 107 aa, synthetic construct [sequence omitted]

SEQ ID NO: 15 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 120 aa, synthetic construct [sequence omitted]

SEQ ID NO: 17 - protein, 111 aa, synthetic construct [sequence omitted]

SEQ ID NO: 18 - protein, 113 aa, synthetic construct [sequence omitted]

SEQ ID NO: 19 - protein, 107 aa, synthetic construct [sequence omitted]

SEQ ID NO: 20 - protein, 120 aa, synthetic construct [sequence omitted]

SEQ ID NO: 21 - protein, 107 aa, synthetic construct [sequence omitted]

SEQ ID NO: 22 - protein, 123 aa, synthetic construct [sequence omitted]

SEQ ID NO: 23 - protein, 107 aa, synthetic construct [sequence omitted]

SEQ ID NO: 24 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 25 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 26 - protein, 122 aa, synthetic construct [sequence omitted]

SEQ ID NO: 27 - protein, 106 aa, synthetic construct [sequence omitted]

SEQ ID NO: 28 - protein, 127 aa, synthetic construct [sequence omitted]

SEQ ID NO: 29 - protein, 108 aa, synthetic construct [sequence omitted]

SEQ ID NO: 30 - protein, 121 aa, synthetic construct [sequence omitted]

SEQ ID NO: 31 - protein, 112 aa, synthetic construct [sequence omitted]

SEQ ID NO: 32 - protein, 122 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 33 - protein, 112 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIK

SEQ ID NO: 34 - protein, 126 aa, synthetic construct [sequence omitted]

SEQ ID NO: 35 - protein, 107 aa, synthetic construct [sequence omitted]

SEQ ID NO: 36 - protein, 116 aa, synthetic construct
QVQLVESGGGVVQPGRSLRLSCAASGFTFNNYGMHWVRQAPGMGLEWVAVIWHDGSDKYYADSVKGRFTISRDNSKNTLYLQMNSLRAEDTAVYYCARNWNLFDYWGQGTLVTVSS

SEQ ID NO: 37 - protein, 108 aa, synthetic construct
DIQMTQSPSSLSASVGDRITITCRASQSISTYLHWYQQKPGKAPKLLIYAASTLQSGVPSRFSGSGSGTDFTLTISSLQPEDFATYYCQQSFSTPPINFGQGTKLEIK

SEQ ID NO: 38 - protein, 118 aa, synthetic construct
EVQLVESGGGLVQPGGSLRLSCAASGFTSSSYAMNWVRQAPGKGLEWVSTISGMGGSTYYADSVKGRFTISRDNSKNTLYLQMNSLRAEDTAVYYCAKRGYPHSFDIWGQGTMVTVSS

SEQ ID NO: 39 - protein, 106 aa, synthetic construct
DIQMTQSPSSLSASVGDRVTITCRASQSISSFLNWYQQKPGKAPKLLIYAASSLQSGVPSRFSGSGSGTDFTLTISSLQPEDFATYYCQQSYSTLTFGQGTRLEIK

SEQ ID NO: 40 - protein, 118 aa, synthetic construct
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARTWFGELYFDYWGQGTLVTVSS

SEQ ID NO: 41 - protein, 113 aa, synthetic construct
DIVMTQSPDSLAVSLGERATINCKSSQSVLYSSNNKNYLAWYQQKPGQPPKLLIYWASTRESGVPDRFSGSGSGTEFTLTITSLQAEDVAVYYCQQYYSSPLTFGGGTKVEIK

SEQ ID NO: 42 - protein, 120 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 43 - protein, 107 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 44 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 45 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 46 - protein, 120 aa, synthetic construct
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 47 - protein, 111 aa, synthetic construct
EIVMTQSPSTLSASVGDRVIITCQASEIIHSWLAWYQQKPGKAPKLLIYLASTLASGVPSRFSGSGSGAEFTLTISSELQPDDFATYYCQNVYLASTNGANFGQGTKLTVL

SEQ ID NO: 48 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 49 - protein, 415 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 50 - protein, 506 aa, synthetic construct
EFARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQDLLVDTM

SEQ ID NO: 51 - protein, 398 aa, synthetic construct
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 52 - protein, 626 aa, synthetic construct
APHLVQVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 53 - protein, 525 aa, synthetic construct
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 54 - protein, 339 aa, synthetic construct
GPHSMRYFETAVSRPGLEEPRYISVGYVDNKEFVRFDSDAENPRYEPRAPWMEQEGPEYWERETQKAKGQEQWFRVSLRNLLGYYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVIVAVLGVLGAMAIIGAVVAFVMKRRRNTGGKGGDYALAPGSQSSEMSLRDCKAR

SEQ ID NO: 55 - protein, 100 aa, synthetic construct
IQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMR

SEQ ID NO: 56 - protein, 1916 aa, synthetic construct
MDAESIRLNNENLWAWLVRLLSKNPEWLSAKLRSFLPTMDLDCSYEPSNPEVIHRQLNRLFAQGMATWKSFINDLCFELDVPLDMEIPLVSIWGPRDEFSKQLGAGEESCPGPQLYHGAKRPFQSYGSSPRRKNSKKQQLELAKKYLKLLKTSAQQWHGGVCPGAWLTPHSPQTYIPPVLQWSRATAPLDAQEGATLGDPEAADNIDVSIQDLFSFKAHKGPRVTVLLGKAGMGKTTLAYRLRWRWAQGQLDRFQALFLFEFRQLNMITQLPTLPQLLFDLYLMPESEPDAVFQYLKENAQEVLLIFDGLDEALHADSVGTDNAGSALTLFSELCHGNLLPGCWVMTTSRPGKLPSCVPTEAATVHMWGFDGLRVEKYVTCFFSDLLSQELALKEMRTNARLRGMCAIPALCTVTCFCLRRLLPGSSPGQSAALLPTITQLYLQMVETFSPSETLLDTSILGFGKVALRGLDTGKVVFSVEDISPQLMSFGAVHSLLTSFCIHTRPGHEEIGYAFVHLSLQEFFAALYLMASHTVDKDTLVEYVTLNSHWVLRTKGRLGLSDHLPAFLAGLASHTCHMFLCQLAQQDRAWVGSRQAAVIQVLRKLASRKLTGPKMIELYHCVAETQDLELARFTAQSLPSRLSFHNFPLTHADLAALANILEHRDDPIHLDFDGCPLEPHCPEALVGCGQVENLSFKSRKCGDAFAEALCRSLPTMGSLKTLGLTGSRITAQGISHLIQTLPLCSQLEEVSLHDNQLKDPEVLSLVELLPSLPKLQKLDLSRNSFSRSILLSLVKVAITCPTVRKLQVRELDLIFYLSPVTETATQQSGASDVQGKDSLKEGQSRSLQLRLQKCQLRIRDAEALVELFQKSPQLEEVNLSGNHLEDDGCRLVAEAASQLHIAQKLDLSDNGLSQTGVTYVLKAMSTCGTLEDLHISLLNNTVVLTFAQEPREQEGSCKGRAPLISFVSPVTSELSQRSRRIRLTHCGFLAKHTETLCEALRASCQTHNLDHLDLSDNSLGGKGVILLTELLPGLGPLKSLNLSRNGLSMDAVFSLVQCLSSLQWVFHLDVSLESDCIFLRGAGTSRDALEPKFQTGVQVLELSQRYTSRSFCLQECQLEPTSLTFLCATLEKSPGPLEVQLSCKSLSDDSLKILLQCLPQLPQLSLLQLRHTVLSSRSPFLLADIFNLCPRVRKVTLRSLCHAVLHFDSNEEQEGVCCGFPGCSLSQEHMETLCCALSKCNALSQLDLTDNLLGDIGLRCLLECLPQLPISGWLDLSHNNISQEGILYLLETLPSYPNIQEVSVSLSSEQIFRMCFSKKEGAGTSLRLCECSFSPEQVSKLASSLSQAQQLTELWLTKCHLDLPQLTMLLNLVNRPTGLLGLRLEEPWVDSVSLPALMEVCAQASGCLTELSISEIQRKLWLQLEFPHQEGNSDSMALRLAHCDLETEHSHLMIQLVETYARLQQLSLSQVSFNDNDGTSSKLLQNILLSSCELKSFRLTFSQVSTKSLTHLAFGLGHCHHLEELDFSNNSLREEDTELLMGALQGTCRLKKLHLSFLPLGASSLALLIQGLSRMTLLQDLCLSHNQIGDVGTQCLAAILPKLPELRKFDLSHNQIGDVGTQCLAAILPKLPELRKFNLSHNQIGHVGTQCLAAILPKLPELRKFDLSRNQIGDVGTQCLAAILPKLPELRKFDLSGNRIGPAGGVQLVKSLTHFEHLEEIKLGNNALGEPTALELAQRLPPQLRVLCLPSSHLGPEGALGLAQALEQCPHIEEVSLAENNLAGGVPRFSKRLPLLRQIDLEFCKIEDQAARHLAANLTLFPALEKLLLSGNLLGDEVAAELAQVLPQMGQLKKVNLEWNRITARGAQLLAQGLVQGSCVPVIRLWNNPILNDVAQSLQSQEPRLDFSITDQQTLR

SEQ ID NO: 57 - protein, 365 aa, synthetic construct
MAVMAPRTLLLLLSGALALTQTWAGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 58 - protein, 99 aa, synthetic construct [sequence omitted]

SEQ ID NO: 59 - protein, 1866 aa, synthetic construct
MDPVGLQLGNKNLWSCLVRLLTKDPEWLNAKMKFFLPNTDLDSRNETLDPEQRVILQLNKLHVQGSDTWQSFIHCVCMQLEVPLDLEVLLLSTFGYDDGFTSQLGAEGKSQPESQLHHGLKRPHQSCGSSPRRKQCKKQQLELAKKYLQLLRTSAQQRYRSQIPGSGQPHAFHQVYVPPILRRATASLDTPEGAIMGDVKVEDGADVSISDLFNTRVNKGPRVTVLLGKAGMGKTTLAHRLCQKWAEGHLNCFQALFLFEFRQLNLITRFLTPSELLFDLYLSPESDHDTVFQYLEKNADQVLLIFDGLDEALQPMGPDGPGPVLTLFSHLCNGTLLPGCRVMATSRPGKLPACLPAEAAMVHMLGFDGPRVEEYVNHFFSAQPSREGALVELQTNGRLRSLCAVPALCQVACLCLHHLLPDHAPGQSVALLPNMTQLYMQMVLALSPPGHLPTSSLLDLGEVALRGLETGKVIFYAKDIAPPLIAFGATHSLLTSFCVCTGPGHQQTGYAFTHLSLQEFLAALHLMASPKVNKDTLTQYVTLHSRWVQRTKARLGLSDHLPTFLAGLASCTCRPFLSHLAQGNEDCVGAKQAAVVQVLKKLATRKLTGPKVVELCHCVDETQEPELASLTAQSLPYQLPFHNFPLTCTDLATLTNILEHREAPIHLDFDGCPLEPHCPEALVGCGQIENLSFKSRKCGDAFAEALSRSLPTMGRLQMLGLAGSKITARGISHLVKALPLCPQLKEVSFRDNQLSDQVVLNIVEVLPHLPRLRKLDLSSNSICVSTLLCLARVAVTCPTVRMLQAREADLIFLLSPPTETTAELQRAPDLQESDGQRKGAQSRSLTLRLQKCQLQVHDAEALIALLQEGPHLEEVDLSGNQLEDEGCRLMAEAASQLHIARKLDLSNNGLSVAGVHCVLRAVSACWTLAELHISLQHKTVIFMFAQEPEEQKGPQERAAFLDSLMLQMPSELPLSSRRMRLTHCGLQEKHLEQLCKALGGSCHLGHLHLDFSGNALGDEGAARLAQLLPGLGALQSLNLSENGLSLDAVLGLVRCFSTLQWLFRLDISFESQHILLRGDKTSRDMWATGSLPDFPAAAKFLGFRQRCIPRSLCLSECPLEPPSLTRLCATLKDCPGPLELQLSCEFLSDQSLETLLDCLPQLPQLSLLQLSQTGLSPKSPFLLANTLSLCPRVKKVDLRSLHHATLHFRSNEEEEGVCCGRFTGCSLSQEHVESLCWLLSKCKDLSQVDLSANLLGDSGLRCLLECLPQVPISGLLDLSHNSISQESALYLLETLPSCPRVREASVNLGSEQSFRIHFSREDQAGKTLRLSECSFRPEHVSRLATGLSKSLQLTELTLTQCCLGQKQLAILLSLVGRPAGLFSLRVQEPWADRARVLSLLEVCAQASGSVTEISISETQQQLCVQLEFPRQEENPEAVALRLAHCDLGAHHSLLVGQLMETCARLQQLSLSQVNLCEDDDASSLLLQSLLLSLSELKTFRLTSSCVSTEGLAHLASGLGHCHHLEELDLSNNQFDEEGTKALMRALEGKWMLKRLDLSHLLLNSSTLALLTHRLSQMTCLQSLRLNRNSIGDVGCCHLSEALRAATSLEELDLSHNQIGDAGVQHLATILPGLPELRKIDLSGNSISSAGGVQLAESLVLCRRLEELMLGCNALGDPTALGLAQELPQHLRVLHLPFSHLGPGGALSLAQALDGSPHLEEISLAENNLAGGVLRFCMELPLLRQIDLVSCKIDNQTAKLLTSSFTSCPALEVILLSWNLLGDEAAAELAQVLPQMGRLKRVDLEKNQITALGAWLLAEGLAQGSSIQVIRLWNNPIPCDMAQHLKSQEPRLDFAFFDNQPQAPWGT

SEQ ID NO: 60 - protein, 313 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 61 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 62 - protein, 883 aa, synthetic construct
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 63 - protein, 1438 aa, synthetic construct
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 64 - protein, 2791 aa, synthetic construct
AEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 65 - protein, 67 aa, synthetic construct
MATTMEQETCAHSLTFEECPKCSALQYRNGFYLLQYDEEWYPEELLTDGEDDVFDPELDMEVVFELQ

SEQ ID NO: 66 - protein, 67 aa, synthetic construct
MATTMEQETCAHSLTFEECPKCSALQYRNGFYLLKYAEEWYPEELLTDGEDDVFDPELDMEVVFELQ

SEQ ID NO: 67 - protein, 67 aa, synthetic construct
MATTMEQETCAHSLTFEECPKCSALQYRNGFYLLKFDEEWYPEELLTDGEDDVFDPELDMEVVFELQ

SEQ ID NO: 68 - protein, 67 aa, synthetic construct
MATTMEQETCAHSLTFEECPKCSALQYRNGFYLLKYDEAWYPEELLTDGEDDVFDPELDMEVVFELQ

SEQ ID NO: 69 - protein, 67 aa, synthetic construct; identical to SEQ ID NO: 2

SEQ ID NO: 70 - DNA, 19 bp, synthetic construct
  source 1..19: mol_type = other DNA
taatacgactcactatagg

SEQ ID NO: 71 - DNA, 46 bp, synthetic construct
  source 1..46: mol_type = other DNA
ggaaataagagagaaaagaagagtaagaagaaatataagagccacc

SEQ ID NO: 72 - DNA, 104 bp, synthetic construct
  source 1..104: mol_type = other DNA
gctgccttctgcggggcttgccttctggccatgcccttcttctctcccttgcacctgtacctcttggtctttgaataaagcctgagtaggaagtgagggtctag

SEQ ID NO: 73 - DNA, 34 bp, synthetic construct
  source 1..34: mol_type = other DNA
aaaaaaaaaaaaaaaaaaaaaaaaaaaaaaaaaa

SEQ ID NO: 74 - DNA, 516 bp, synthetic construct
  source 1..516: mol_type = other DNA
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtag

SEQ ID NO: 75 - protein, 190 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAAAANWQAAYIELAAIGMPP

SEQ ID NO: 76 - DNA, 3822 bp, synthetic construct
  source 1..3822: mol_type = other DNA
atgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacctag

SEQ ID NO: 77 - DNA, 117 bp, synthetic construct
  source 1..117: mol_type = other DNA
atggagttttcaagtccttccagagaggaatgtcccaagcctttgagtagggtaagcatcatggctggcagcctcacaggattgcttctacttcaggcagtgtcgtgggcatcaggt

SEQ ID NO: 78 - DNA, 1491 bp, synthetic construct
  source 1..1491: mol_type = other DNA
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccag

SEQ ID NO: 79 - DNA, 44 bp, synthetic construct
  source 1..44: mol_type = other DNA
atgggcggcgcatgagagaagcccagaccaattacctacccaaa

SEQ ID NO: 80 - DNA, 7569 bp, synthetic construct
  source 1..7569: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcat

SEQ ID NO: 81 - DNA, 1653 bp, synthetic construct
  source 1..1653: mol_type = other DNA
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtag

SEQ ID NO: 82 - DNA, 565 bp, synthetic construct
  source 1..565: mol_type = other DNA
tcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 83 - DNA, 204 bp, synthetic construct
  source 1..204: mol_type = other DNA
atggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 84 - DNA, 298 bp, synthetic construct
  source 1..298: mol_type = other DNA
gaattggcaagctgcttacatagaactcgcggcgattggcatgccgccttaaaatttttattttattttttcttttcttttccgaatcggattttgtttttaatatttcaaaaaaaaaaaaaaaaaaaaaaaaacgcgaaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaattaataaaaaaaaaaaaaaaaaaaaaaaaaaaaaaaaaaaaaaaaaaaaaaaaaa

SEQ ID NO: 85 - DNA, 85 bp, synthetic construct
  source 1..85: mol_type = other DNA
atggcggatgtgtgacatacacgacgccaaaagattttgttccagctcctgccacctcggctacgcgagagattaaccacccacg

SEQ ID NO: 86 - DNA, 7334 bp, synthetic construct
  source 1..7334: mol_type = other DNA
atggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccattccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaggaggcaaacgaacggatatgcctatacgcgctgggcgaaacaatggacaacatcggatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggccgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatgaccaccttggcgagggacattaaggcgtttaagaaattgagaggacctgttatacacctctacggcggtcctagattggtgcgttaatacacagaattctgattatagcgcactattatagcacc

SEQ ID NO: 87 - DNA, 699 bp, synthetic construct
  source 1..699: mol_type = other DNA
atggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagataa

SEQ ID NO: 88 - DNA, 565 bp, synthetic construct; identical to SEQ ID NO: 82
  source 1..565: mol_type = other DNA

SEQ ID NO: 89 - DNA, 204 bp, synthetic construct; identical to SEQ ID NO: 83
  source 1..204: mol_type = other DNA

SEQ ID NO: 90 - DNA, 343 bp, synthetic construct
  source 1..343: mol_type = other DNA
acgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcg

SEQ ID NO: 91 - DNA, 471 bp, synthetic construct
  source 1..471: mol_type = other DNA
tatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataagttagggtaggcaatggcattgatatagcaagaaaattgaaaacagaaaaagttagggtaagcaatggcatataaccataactgtataacttgtaacaaagcgcaacaagacctgcgcaattggccccgtggtccgcctcacggaaactcggggcaactcatattgacacattaattggcaataattggaagcttacataagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaa

SEQ ID NO: 92 - DNA, 44 bp, synthetic construct; identical to SEQ ID NO: 79
  source 1..44: mol_type = other DNA

SEQ ID NO: 93 - DNA, 7569 bp, synthetic construct; identical to SEQ ID NO: 80
  source 1..7569: mol_type = other DNA

SEQ ID NO: 94 - DNA, 1701 bp, synthetic construct
  source 1..1701: mol_type = other DNA
atgaaggccatcctggtggtgctgctgtacaccttcgccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgagaggcgtggcccctctgcacctgggcaagtgcaacatcgccggctggatcctgggcaaccctgagtgcgagagcctgagcaccgctagcagctggagctacatcgtggagacccctagcagcgacaacggcacctgcttccctggcgacttcatcgactacgaggagctgagagagcagctgagcagcgtgagcagcttcgagagattcgagatcttccctaagacaagcagctggcctaatcatgacagtaataagggagtgaccgccgcctgccctcacgccggcgccaagagcttctacaagaacctgatctggctggtgaaggagggcaacagctaccctaagctgagcaagagctacatcaacgacaagggcaaagaggtgctggtgctgtggggcatccaccaccctagcacaagcgccgatcagcagagcctgtatcagaacgccgacgcctacgtgttcgtgggcagcagcagatacagcaagaagttcaagcctgagatcgccatcagacctaaggtgagaggccaagagggcagaatgaactactactggaccctggtggagcctggcgacaagatcaccttcgaggccaccggcaacctggtggtgcctagatacgccttcgccatggagagaaacgccggcagcggcatcatcatcagcgacacccctgtgcacgactgcaacaccacctgtcagacccctaagggcgccatcaacacaagcctgccttttcagaacatccaccctatcaccatcggcaagtgccctaagtacgtgaaaagtaccaagctgagactggccaccggcctgagaaacatccctagcattcagagcagaggcctgttcggcgccatcgccggcttcatcgagggcggctggaccggcatggtggacggctggtacggctaccaccatcagaacgagcaaggaagcggctacgccgccgacctgaaaagtacacagaacgccatcgacgagatcaccaacaaggtgaacagcgtgatcgagaagatgaacacacagttcaccgccgtgggcaaggagttcaaccacctggagaagagaatcgagaacctgaacaagaaggtggacgacggcttcctggacatctggacctacaacgccgagctgctggtgctgctggagaacgagagaaccctggactaccacgacagtaatgttaaaaacctgtacgagaaggtgagatctcagctgaagaacaacgccaaggagatcggcaacggctgcttcgagttctaccacaagtgcgacaacacctgcatggagagcgtgaagaacggcacctacgactaccctaagtacagcgaggaggccaagctgaacagagaggagatcgacggcgtgaagctggagagcacaagaatctatcagatcctggccatctacagcaccgtggctagcagcctggtgctggtggtgagcctgggcgccatcagcttctggatgtgcagcaacggcagcctgcagtgcagaatctgcatctag

SEQ ID NO: 95 - DNA, 565 bp, synthetic construct; identical to SEQ ID NO: 82
  source 1..565: mol_type = other DNA

SEQ ID NO: 96 - DNA, 204 bp, synthetic construct; identical to SEQ ID NO: 83
  source 1..204: mol_type = other DNA

SEQ ID NO: 97 - DNA, 298 bp, synthetic construct; identical to SEQ ID NO: 84
  source 1..298: mol_type = other DNA

SEQ ID NO: 98 - DNA, 50 bp, synthetic construct
  source 1..50: mol_type = other DNA
acatttgcttctgacacaactgtgttcactagcaacctcaaacagacacc

SEQ ID NO: 99 - DNA, 1071 bp, synthetic construct
  source 1..1071: mol_type = other DNA
atggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttg

SEQ ID NO: 100 - DNA, 45 bp, synthetic construct
  source 1..45: mol_type = other DNA
ggaggcagcggcggaggaagcggtggaggctctggaggcggaagc

SEQ ID NO: 101 - DNA, 588 bp, synthetic construct
  source 1..588: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagatgtggaggagaaccctggacctatagaacaatag

SEQ ID NO: 102 - DNA, 134 bp, synthetic construct
  source 1..134: mol_type = other DNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 103 - DNA, 50 bp, synthetic construct; identical to SEQ ID NO: 98
  source 1..50: mol_type = other DNA

SEQ ID NO: 104 - DNA, 1254 bp, synthetic construct
  source 1..1254: mol_type = other DNA
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagaggccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaa

SEQ ID NO: 105 - DNA, 45 bp, synthetic construct; identical to SEQ ID NO: 100
  source 1..45: mol_type = other DNA

SEQ ID NO: 106 - DNA, 588 bp, synthetic construct; identical to SEQ ID NO: 101
  source 1..588: mol_type = other DNA

SEQ ID NO: 107 - DNA, 134 bp, synthetic construct; identical to SEQ ID NO: 102
  source 1..134: mol_type = other DNA

SEQ ID NO: 108 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 109 - DNA, 110 bp, synthetic construct
  source 1..110: mol_type = other DNA
ggtttagtgaaccgtcagatccgctagcgctaccggactcagatctcgagctcaagcttcgaattctgcagtcgacggtaccgcgggcccgggatccaccggtcgccacc

SEQ ID NO: 110 - DNA, 1608 bp, synthetic construct
  source 1..1608: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccag

SEQ ID NO: 111 - DNA, 12 bp, synthetic construct
  source 1..12: mol_type = other DNA
ggctcgagcggc

SEQ ID NO: 112 - DNA, 513 bp, synthetic construct
  source 1..513: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 113 - DNA, 248 bp, synthetic construct
  source 1..248: mol_type = other DNA
ttacagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 114 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 115 - DNA, 89 bp, synthetic construct
  source 1..89: mol_type = other DNA
ggtttagtgaaccgtcagatcagatctttgtcgatcctaccatccactcgacacacccgccagcggccgctgccaagcttgccgccacc

SEQ ID NO: 116 - DNA, 579 bp, synthetic construct
  source 1..579: mol_type = other DNA
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacaga

SEQ ID NO: 117 - DNA, 45 bp, synthetic construct; identical to SEQ ID NO: 100
  source 1..45: mol_type = other DNA

SEQ ID NO: 118 - DNA, 513 bp, synthetic construct; identical to SEQ ID NO: 112
  source 1..513: mol_type = other DNA

SEQ ID NO: 119 - DNA, 171 bp, synthetic construct
  source 1..171: mol_type = other DNA
gctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 120 - DNA, 44 bp, synthetic construct; identical to SEQ ID NO: 79
  source 1..44: mol_type = other DNA

SEQ ID NO: 121 - DNA, 1605 bp, synthetic construct
  source 1..1605: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggcc

SEQ ID NO: 122 - DNA, 2382 bp, synthetic construct
  source 1..2382: mol_type = other DNA
ggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaattcttacaagctttcatcaaccttgaccaacatttatacaggttccagactccacgaagccggatgt

SEQ ID NO: 123 - DNA, 1671 bp, synthetic construct
  source 1..1671: mol_type = other DNA
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 124 - DNA, 1867 bp, synthetic construct
  source 1..1867: mol_type = other DNA
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagtctagcat

SEQ ID NO: 125 - DNA, 54 bp, synthetic construct
  source 1..54: mol_type = other DNA
atgaagtgggtgaccttcatcagcctgctgttcctgttttcttccgcctacagc

SEQ ID NO: 126 - DNA, 513 bp, synthetic construct
  source 1..513: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtag

SEQ ID NO: 127 - DNA, 565 bp, synthetic construct; identical to SEQ ID NO: 82
  source 1..565: mol_type = other DNA

SEQ ID NO: 128 - DNA, 204 bp, synthetic construct; identical to SEQ ID NO: 83
  source 1..204: mol_type = other DNA

SEQ ID NO: 129 - DNA, 298 bp, synthetic construct; identical to SEQ ID NO: 84
  source 1..298: mol_type = other DNA

SEQ ID NO: 130 - DNA, 44 bp, synthetic construct; identical to SEQ ID NO: 79
  source 1..44: mol_type = other DNA

SEQ ID NO: 131 - DNA, 1605 bp, synthetic construct; identical to SEQ ID NO: 121
  source 1..1605: mol_type = other DNA

SEQ ID NO: 132 - DNA, 2382 bp, synthetic construct; identical to SEQ ID NO: 122
  source 1..2382: mol_type = other DNA

SEQ ID NO: 133 - DNA, 1671 bp, synthetic construct; identical to SEQ ID NO: 123
  source 1..1671: mol_type = other DNA

SEQ ID NO: 134 - DNA, 1867 bp, synthetic construct; identical to SEQ ID NO: 124
  source 1..1867: mol_type = other DNA

SEQ ID NO: 135 - DNA, 54 bp, synthetic construct; identical to SEQ ID NO: 125
  source 1..54: mol_type = other DNA

SEQ ID NO: 136 - DNA, 513 bp, synthetic construct; identical to SEQ ID NO: 126
  source 1..513: mol_type = other DNA

SEQ ID NO: 137 - DNA, 565 bp, synthetic construct; identical to SEQ ID NO: 82
  source 1..565: mol_type = other DNA

SEQ ID NO: 138 - DNA, 204 bp, synthetic construct
  source 1..204: mol_type = other DNA
atggccacaaccatggaacaagagacttgcgcgcactctctcacttttgaggaatgcccaaaatgctctgctctacaataccgtaatggattttacctgctaaagtatgatgaagcatggtacccagaggagttattgactgatggagaggatgatgtctttgatcccgaattagacatggaagtcgttttcgagttacagtaa

SEQ ID NO: 139 - DNA, 298 bp, synthetic construct; identical to SEQ ID NO: 84
  source 1..298: mol_type = other DNA

SEQ ID NO: 140 - DNA, 44 bp, synthetic construct; identical to SEQ ID NO: 79
  source 1..44: mol_type = other DNA

SEQ ID NO: 141 - DNA, 1605 bp, synthetic construct; identical to SEQ ID NO: 121
  source 1..1605: mol_type = other DNA

SEQ ID NO: 142 - DNA, 2382 bp, synthetic construct; identical to SEQ ID NO: 122
  source 1..2382: mol_type = other DNA

SEQ ID NO: 143 - DNA, 1671 bp, synthetic construct; identical to SEQ ID NO: 123
  source 1..1671: mol_type = other DNA

SEQ ID NO: 144 - DNA, 1867 bp, synthetic construct; identical to SEQ ID NO: 124
  source 1..1867: mol_type = other DNA

SEQ ID NO: 145 - DNA, 54 bp, synthetic construct; identical to SEQ ID NO: 125
  source 1..54: mol_type = other DNA

SEQ ID NO: 146 - DNA, 513 bp, synthetic construct; identical to SEQ ID NO: 126
  source 1..513: mol_type = other DNA

SEQ ID NO: 147 - DNA, 565 bp, synthetic construct; identical to SEQ ID NO: 82
  source 1..565: mol_type = other DNA

SEQ ID NO: 148 - DNA, 204 bp, synthetic construct
  source 1..204: mol_type = other DNA
atggccacaaccatggaacaagagacttgcgcgcactctctcacttttgaggaatgcccaaaatgctctgctctacaataccgtaatggattttacctgctacagtatgatgaagaatggtacccagaggagttattgactgatggagaggatgatgtctttgatcccgaattagacatggaagtcgttttcgagttacagtaa

SEQ ID NO: 149 - DNA, 298 bp, synthetic construct; identical to SEQ ID NO: 84
  source 1..298: mol_type = other DNA

SEQ ID NO: 150 - DNA, 44 bp, synthetic construct; identical to SEQ ID NO: 79
  source 1..44: mol_type = other DNA

SEQ ID NO: 151 - DNA, 1605 bp, synthetic construct; identical to SEQ ID NO: 121
  source 1..1605: mol_type = other DNA

SEQ ID NO: 152 - DNA, 1605 bp, synthetic construct; identical to SEQ ID NO: 121
  source 1..1605: mol_type = other DNA

SEQ ID NO: 153 - DNA, 1671 bp, synthetic construct; identical to SEQ ID NO: 123
  source 1..1671: mol_type = other DNA

SEQ ID NO: 154 - DNA, 1867 bp, synthetic construct; identical to SEQ ID NO: 124
  source 1..1867: mol_type = other DNA

SEQ ID NO: 155 - DNA, 54 bp, synthetic construct; identical to SEQ ID NO: 125
  source 1..54: mol_type = other DNA

SEQ ID NO: 156 - DNA, 513 bp, synthetic construct; identical to SEQ ID NO: 126
  source 1..513: mol_type = other DNA

SEQ ID NO: 157 - DNA, 565 bp, synthetic construct; identical to SEQ ID NO: 82
  source 1..565: mol_type = other DNA

SEQ ID NO: 158 - DNA, 192 bp, synthetic construct
  source 1..192: mol_type = other DNA
atagaacaatagacttgagctgactagctgacttttgaggaatgaccaaaatgatctgctctagaataccgtaatggattttagctgctaaagtaggatgaagaatggtacccagaggagttattgactgatggagaggatgatgtctttgatcccgaattagacatggaagtcgttttcgagttacagtaa

SEQ ID NO: 159 - DNA, 298 bp, synthetic construct; identical to SEQ ID NO: 84
  source 1..298: mol_type = other DNA

SEQ ID NO: 160 - RNA, 204 nt, synthetic construct
  source 1..204: mol_type = other RNA
atggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 161 - protein, 67 aa, synthetic construct; identical to SEQ ID NO: 2

SEQ ID NO: 162 - RNA, 204 nt, synthetic construct
  source 1..204: mol_type = other RNA
atggccacaaccatggaacaagagacttgcgcgcactctctcacttttgaggaatgcccaaaatgctctgctctacaataccgtaatggattttacctgctaaagtatgatgaagcatggtacccagaggagttattgactgatggagaggatgatgtctttgatcccgaattagacatggaagtcgttttcgagttacagtaa

SEQ ID NO: 163 - protein, 67 aa, synthetic construct; identical to SEQ ID NO: 68

SEQ ID NO: 164 - RNA, 201 nt, synthetic construct
  source 1..201: mol_type = other RNA
atggccacaaccatggaacaagagacttgcgcgcactctctcacttttgaggaatgcccaaaatgctctgctctacaataccgtaatggattttacctgctacagtatgatgaagaatggtacccagaggagttattgactgatggagaggatgatgtctttgatcccgaattagacatggaagtcgttttcgagttacag

SEQ ID NO: 165 - protein, 67 aa, synthetic construct; identical to SEQ ID NO: 65

SEQ ID NO: 166 - RNA, 204 nt, synthetic construct; identical to SEQ ID NO: 160
  source 1..204: mol_type = other RNA

SEQ ID NO: 167 - protein, 67 aa, synthetic construct; identical to SEQ ID NO: 67

SEQ ID NO: 168 - RNA, 204 nt, synthetic construct; identical to SEQ ID NO: 160
  source 1..204: mol_type = other RNA

SEQ ID NO: 169 - protein, 67 aa, synthetic construct; identical to SEQ ID NO: 66

SEQ ID NO: 170 - RNA, 192 nt, synthetic construct
  source 1..192: mol_type = other RNA
atagaacaatagacttgagctgactagctgacttttgaggaatgaccaaaatgatctgctctagaataccgtaatggattttagctgctaaagtaggatgaagaatggtacccagaggagttattgactgatggagaggatgatgtctttgatcccgaattagacatggaagtcgttttcgagttacagtaa

SEQ ID NO: 171 - protein, 5 aa, synthetic construct
LTFEE

SEQ ID NO: 172 - protein, 9 aa, synthetic construct
SALEYRNGF

SEQ ID NO: 173 - protein, 31 aa, synthetic construct
DEEWYPEELLTDGEDDVFDPELDMEVVFELQ

SEQ ID NO: 174 - RNA, 46 nt, synthetic construct
  source 1..46: mol_type = other RNA
ggaaataagagagaaaagaagagtaagaagaaatataagagccacc

SEQ ID NO: 175 - RNA, 100 nt, synthetic construct
  source 1..100: mol_type = other RNA
gctgccttctgcggggcttgccttctggccatgcccttcttctctcccttgcacctgtacctcttggtctttgaataaagcctgagtaggaagtgagggt

SEQ ID NO: 176 - RNA, 566 nt, synthetic construct
  source 1..566: mol_type = other RNA
tcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataata

SEQ ID NO: 177 - RNA, 470 nt, synthetic construct
  source 1..470: mol_type = other RNA
ggaaataagagagaaaagaagagtaagaagaaatataagagccaccatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaagctgccttctgcggggcttgccttctggccatgcccttcttctctcccttgcacctgtacctcttggtctttgaataaagcctgagtaggaagtgagggtaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0018024 A1
(43) Pub. Date: Jan. 16, 2025

(54) COMPOSITIONS AND METHODS FOR PROTEIN EXPRESSION WITH RNA

(71) Applicant: ExcepGen Inc, San Francisco, CA (US)

(72) Inventors: Barbara Mertins, San Francisco, CA (US); Thomas Folliard, San Francisco, CA (US); Imre Mäger, San Francisco, CA (US); Jason Wojcechowskyj, San Francisco, CA (US)

(21) Appl. No.: 18/893,827

(22) Filed: Sep. 23, 2024

Related U.S. Application Data
(63) Continuation of application No. PCT/US2023/064883, filed on Mar. 23, 2023.
(60) Provisional application No. 63/323,332, filed on Mar. 24, 2022.
(60) Provisional application No. 63/329,050, filed on Apr. 8, 2022.
(60) Provisional application No. 63/375,350, filed on Sep. 12, 2022.
(60) Provisional application No. 63/478,633, filed on Jan. 5, 2023.

(51) Int. Cl. A61K 39/125; A61K 39/00; A61P 29/00; A61P 37/06; C12N 7/00

(52) U.S. Cl. CPC A61K 39/125; A61P 29/00; A61P 37/06; C12N 7/00; A61K 2039/53; C12N 2770/32034

(57) ABSTRACT

The compositions and methods provided herein include a ribonucleic acid (RNA) encoding a nuclear cytoplasmic transport (NCT) inhibitor protein to improve target protein expression, e.g., a target protein encoded by a DNA vector, a messenger RNA, a self-amplifying RNA, or an RNA comprising an unmodified uridine nucleotide. The compositions and methods provided herein may be used to improve the expression of any target protein, for example a viral protein antigen for use in a vaccine.

### CROSS REFERENCE TO RELATED APPLICATIONS

This application is a continuation of International Application No. PCT/US2023/064883, filed on Mar. 23, 2023, which claims priority to U.S. Provisional Patent Application No. 63/323,332, filed on Mar. 24, 2022; U.S. Provisional Patent Application No. 63/329,050, filed on Apr. 8, 2022; U.S. Provisional Patent Application No. 63/375,350, filed on Sep. 12, 2022; and U.S. Provisional Patent Application No. 63/478,633, filed on Jan. 5, 2023, the contents of each which are incorporated herein by reference in their entireties.

### INCORPORATION BY REFERENCE OF SEQUENCE LISTING

The contents of the electronic sequence listing (EXCI_004_04WO_SeqList_ST26.xml; Size: 258,708 bytes; and Date of Creation: Mar. 23, 2023) are herein incorporated by reference in their entirety.

### BACKGROUND

Therapeutic applications of target protein expression for disease treatment and prevention have advanced considerably in recent years. For example, exogenous proteins have been designed to replace abnormal or deficient proteins for a multitude of diseases and to induce immune responses as vaccines. However, long-standing challenges remain to improve target protein expression: exogenous ribonucleic acid (RNA) and deoxyribonucleic acid (DNA) elicit cell stress signals which directly inhibit protein production; RNA is rapidly degraded in the cell resulting in poor protein expression levels; and DNA typically results in over-expression of a protein. Therefore, a need exists for compositions and methods that overcome these challenges and improve target protein expression.

### SUMMARY

Provided herein are compositions comprising an RNA encoding a nucleocytoplasmic transport (NCT) inhibitor protein and a deoxyribonucleic acid (DNA) or a ribonucleic acid (RNA) encoding a target protein. Also provided are methods for improving expression of a target protein with an RNA encoding an NCT inhibitor protein.

In some aspects, a composition of the disclosure comprises a self-amplifying ribonucleic acid (saRNA) encoding a nucleocytoplasmic transport (NCT) inhibitor protein. In some aspects, the RNA encoding the target protein and/or the RNA encoding the NCT inhibitor protein is a messenger RNA (mRNA).

In some aspects, the saRNA encoding the NCT inhibitor protein encodes the target protein. In some aspects, the composition comprising an saRNA encoding an NCT inhibitor protein comprises an mRNA encoding the target protein.

In some aspects, the mRNA or saRNA encoding the target protein comprises at least one unmodified uridine nucleotide.

In some embodiments, the saRNA comprises a sequence of any one of SEQ ID NOS: 79-97, or a sequence with at least 70%, at least 75%, at least 80%, at least 85%, at least 90%, at least 95%, at least 96%, at least 97%, at least 98%, or at least 99% sequence identity thereto.

In some embodiments, the NCT inhibitor protein comprises a sequence of Table 4, a sequence of SEQ ID NOS: 65, 66, 67, or 68, or a sequence with at least 70%, at least 75%, at least 80%, at least 85%, at least 90%, at least 95%, at least 96%, at least 97%, at least 98%, or at least 99% sequence identity thereto.

In some embodiments, the target protein is a viral antigen. In some embodiments, the viral antigen is selected from Table 3.

In some embodiments, the composition comprises a delivery vehicle. In some embodiments, the delivery vehicle is a lipid nanoparticle (LNP).

In some aspects, provided herein is a method of improving the expression of a target protein, comprising expressing a ribonucleic acid (RNA) encoding a nucleocytoplasmic transport (NCT) inhibitor protein, wherein the target protein is encoded by a ribonucleic acid (RNA) or a deoxyribonucleic acid (DNA).

In some embodiments of the method, the RNA encoding the target protein is a self-amplifying ribonucleic acid (saRNA). In some embodiments of the method, the RNA encoding the target protein is a messenger ribonucleic acid (mRNA). In some embodiments of the method, the RNA encoding the NCT inhibitor protein is a messenger RNA (mRNA). In some embodiments of the method, the saRNA encoding the target protein also encodes the NCT inhibitor protein.

In some embodiments of the method, the RNA encoding the target protein comprises at least one unmodified uridine nucleotide.

In some embodiments of the method, expression of the NCT inhibitor protein increases the target protein expression level by about 2-fold, about 3-fold, about 4-fold, about 5-fold, about 6-fold, about 7-fold, about 8-fold, about 9-fold, about 10-fold, about 20-fold, about 30-fold, about 40-fold, 50-fold, about 60-fold, about 70-fold, about 80-fold, about 90-fold, about 100-fold, about 200-fold, about 500 fold, or about 1000-fold.

In some embodiments of the method, expression of the NCT inhibitor protein reduces the expression level of the target protein by about 10%, about 20%, about 30%, about 40%, about 50%, about 60%, about 70%, about 80%, or about 90%.

In some embodiments of the method, expression of the NCT inhibitor protein increases the activity of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments of the method, expression of the NCT inhibitor protein reduces the EC50 of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments of the method, the NCT inhibitor protein and the target protein are expressed in a cell or subject. In some embodiments of the method, the NCT inhibitor protein increases the duration of time in which the target protein is found in the cell or subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20-fold.

In some embodiments of the method, co-expression of the NCT inhibitor protein decreases the coefficient of variation (CV %) of the target protein in the tissue of the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

In some embodiments of the method, the NCT inhibitor protein comprises a sequence of Table 4, a sequence of SEQ ID NOS: 65, 66, 67, or 68, or a sequence with at least 70%, at least 75%, at least 80%, at least 85%, at least 90%, at least 95%, at least 96%, at least 97%, at least 98%, or at least 99% sequence identity thereto.

In some embodiments of the method, the target protein is a viral antigen.

In some aspects, provided herein is a method of reducing cell signaling in response to a stressor comprising introducing a polynucleotide encoding an NCT inhibitor to a cell.

In some embodiments of the method, the cell signaling in response to a stressor is expression of interferon-beta, and introducing the NCT inhibitor reduces interferon-beta expression in the cell by about 10-fold, about 100-fold, about 1000-fold, about 10,000 fold, or about 100,000 fold.

### BRIEF DESCRIPTION OF THE DRAWINGS

FIGS. 1A-1E show example DNA templates for RNA preparation.

FIGS. 2A and 2B show RNA constructs encoding luciferase. FIG. 2A shows an example RNA encoding luciferasc. FIG. 2B shows an example RNA encoding an NCT inhibitory protein that may be co-expressed with the RNA of FIG. 2A.

FIGS. 3A and 3B show RNA constructs encoding a Sars-CoV2 Spike protein. FIG. 3A shows an example RNA encoding a Sars-CoV2 Spike protein. FIG. 3B shows an example RNA encoding an NCT inhibitory protein that may be co-expressed with the RNA of FIG. 3A.

FIGS. 4A and 4B show RNA constructs encoding Glucosylceramidasc (GBA) FIG. 4A shows an example RNA encoding GBA protein. FIG. 4B shows an example RNA encoding an NCT inhibitory protein that may be co-expressed with the RNA of FIG. 4A.

FIGS. 5A and 5B show DNA construct configurations used for in vitro transcription (IVT) to generate single mRNA constructs encoding the L protein (L) and nano luciferase (nLuc). FIG. 5A shows a DNA construct used for IVT to generate a single mRNA construct encoding L and nLuc separated by a p2A separating element (L-NLuc). FIG. 5B shows a DNA construct used for IVT to generate a single mRNA construct encoding nLuc and L separated by a p2A separating element (NLuc-L).

FIGS. 6A and 6B show luciferase activity assays when luciferase is expressed from mammalian cells 24 hours, 48 hours, and 72 hours post transfection. Measurements were done in quadruplicates bar charts to present the mean. Error bars indicate standard deviation. The figures show total luciferase (nLuc) activity measured in relative light units (RLU) with transfection of mRNA encoding either the L protein alone (L), nano Luficerase (nLuc) alone, or the two configurations of a single mRNA encoding L and nLuc as shown in FIGS. 5A (L-nLuc) and 5B (nLuc-L). These mRNA contained about 50% natural and about 50% pseudouridine. FIG. 6A shows the result of mouse C2C12 cell transfection. The presence of L protein in combination with nLuc showed an increased activity as result of higher expression of nLuc in the presence of L protein by up to 3.5 fold or 5.5 fold for L-nLuc and nLuc-L respectively after 72 hours. FIG. 6B shows the result from transfection of human BJ cells. The effect from the presence of L protein in combination with nLuc shows more pronounced increased activity as result of higher expression of nLuc in the presence of L by up to 6.5 fold or 14 fold for L-nLuc and nLuc-L respectively after 72 hours in the presence of mRNA containing about 50% natural uridine.

FIG. 7 shows luciferase activity assays expressed from mammalian cells in the presence of an external source of interferon (IFN) beta. C2C12 cells were transfected with mRNA encoding either the L protein alone (L), nLuc alone, or the two configurations of a single mRNA encoding L and nLuc as shown in FIGS. 5A (L-nLuc) and 5B (nLuc-L). These mRNA contained about 50% natural and about 50% pseudouridine. 6 hours post transfection, a 10 fold dilution series of IFN-beta was added to the cells. Luciferase activity was then measured 48 hours after IFN-beta stimulation. The graph shows decreased luciferase activity as a result of decreased luciferase expression in the presence of IFN-beta (as a percentage of the total amount of expression of nLuc in the absence of IFN-beta). The approximate EC50 value of IFN-beta required to decrease luciferase expression for each construct is shown, as indicated by the dotted line. The presence of the L protein protects cells from the presence of external IFN-beta 10-fold and 12 fold (for L-nLuc and nLuc-L, respectively) as demonstrated by the increased approximate EC50 values.

FIG. 8 shows IFN-beta production of cells as an indication of the cell internal stress response. mRNA encoding the L protein alone (L), nLuc alone, or the two configurations of a single mRNA encoding L and nLuc as shown in FIGS. 5A (L-nLuc) and 5B (nLuc-L) were transfected in human BJ cells. These mRNA contained about 50% natural and about 50% pseudouridine. The presence of the protein L inhibited IFN-beta production in the cells to baseline level.

FIG. 9 shows the effects of mRNA based L protein expression on Poly I:C induced down-regulation of gene of interest expression.

FIG. 10 shows the effects of mRNA based L protein expression on IFN-beta induced down-regulation of gene of interest expression.

FIGS. 11A-11C show the effects of mRNA based L protein expression on natural nucleoside induced down-regulation of gene of interest expression. FIG. 11A shows the effects of L protein on gene of interest expression from mRNAs containing only natural nucleosides, including natural uridine, using different concentrations of L protein. FIG. 11B shows the effect of L protein expression on modified mRNA containing only non-inflammatory nucleosides, including pseudo-uridine, using different concentrations of L protein. FIG. 11C compares the effects of the L protein over a time course of 20 days post transfection on gene of interest expression from natural nucleoside-only mRNA and modified mRNA.

FIGS. 12A and 12B show the effects of L protein expression on gene of interest expression with self-amplifying RNA (saRNA). FIG. 12A shows the effects of L protein expression on gene of interest expression with saRNA, using different concentrations of L protein. FIG. 12B compares the effects of different concentrations of L protein over a time course of 20 days post transfection on gene of interest expression from saRNA.

FIG. 13 shows the effects of L protein expression on gene of interest expression with self-amplifying RNA (saRNA), using mutated forms of the L protein (SEQ ID NOS: 65, 66, 67, 68) at different concentrations on day 6 after transfection.

FIG. 14 shows the effects of L protein expression on gene of interest expression with self-amplifying RNA (saRNA), using mutated forms of the L protein (SEQ ID NOS: 65, 66, 67, 68) at different concentrations on day 9 after transfection.

FIGS. 15A and 15B show the effects of L protein, and self-amplifying RNA (saRNA) expressing of a gene interest, on the associated expression of inflammation mediators IFN-beta (FIG. 15A) and IP-10 (FIG. 15B).

FIG. 16 shows the effects of L protein expression on self-amplifying RNA (saRNA) based gene of interest expression over time in vivo. saRNA expression of the L protein increased significantly target protein expression by the saRNA construct.

FIG. 17 shows the effects of L protein expression on tetherin expression in the presence of IFN-beta. Tetherin is downstream of IFN-beta and is a negative regulator of VLP production.

FIGS. 18A and 18B show mRNA expression vectors in which the genes of interest (GOIs) were tagged with the NanoLuc reporter protein via a linker peptide. The coding sequences of G6PC1 (FIG. 18A, SEQ ID NO: 99) and SERPINA1 (FIG. 18B, SEQ ID NO: 104) were flanked by hBG 5′ UTR (SEQ ID NO: 98) and hBG 3′UTR (SEQ ID NO: 102). mRNA expression vectors contained the Cap1 mRNA cap structure, N1-methylpseudouridine modifications and a 120-nt polyA tail. mRNAs were produced using in vitro transcription (IVT).

FIGS. 19A-19D show the expression of G6PC1 and SERPINA1 mRNA in cell lysates. Each experiment was performed using three technical replicates. A total of five independent experiments (biological replicates) were performed. FIG. 19A shows G6PC1 expression from L protein expression (EG Tech) in relative light units (RLU). FIG. 19B shows G6PC1 expression from L protein expression (EG Tech) as a fold change relative to no L protein. FIG. 19C shows SERPINA1 expression from L protein expression (EG Tech) in relative light units (RLU). FIG. 19D shows SERPINA1 expression from L protein expression (EG Tech) as a fold change relative to no L protein.

FIGS. 20A and 20B show the plasmid DNA expression vectors encoding a lysosomal acid glucosylceramidase (GBA) gene (FIG. 20A) and an erythropoietin (EPO) gene (FIG. 20B), each tagged with the NanoLuc reporter protein via a peptide linker and driven by a CMV promoter.

FIG. 21 shows GBA expression 48 hours after transfection with the DNA expression vector of FIG. 20A and an mRNA encoding the L protein (EG Tech). The stimulation of cells with pro-inflammatory molecules IFN-beta and Poly I:C reduced GBA expression. However, in IFN-beta and Poly I:C stimulated cells, L protein mRNA expression increased GBA expression relative to no L protein.

FIG. 22 shows CCL2 induction in GBA-expressing plasmid DNA-transfected cells. CCL2 induction was reduced when L protein mRNA was also added to the cells.

FIG. 23A-23E shows cells transfected with the GBA-encoding plasmid DNA of FIG. 20A and also stimulated with Poly I:C to induce a high level of secretion of inflammation related molecules CCL2 (FIG. 23A), CCL5 (FIG. 23B), CXCL10 induction (FIG. 24C), IFN-alpha (FIG. 23D) and IFN-beta (FIG. 23E). However, in the presence of L protein mRNA, the level of each inflammation molecule was reduced to the baseline levels of untransfected and non-stimulated cells.

FIGS. 24A-24E shows cells transfected with the EPO-encoding plasmid DNA of FIG. 20B and also stimulated with Poly I:C to induce a high level of secretion of inflammation related molecules CCL2 (FIG. 24A), CCL5 (FIG. 24B), CXCL10 (FIG. 24C), IFN-alpha (FIG. 24D) and IFN-beta (FIG. 24E). However, in the presence of L protein mRNA, the level of each inflammation molecule was reduced to the baseline of untransfected and non-stimulated cells.

FIGS. 25A-25C show self-amplifying RNA vector constructs encoding the L protein coding sequence (FIG. 25A), an L protein mutant coding sequence (FIG. 25B), or a scrambled negative control sequence (FIG. 25C) together with a NanoLuciferase reporter gene.

FIG. 26 shows self-amplifying RNA construct mediated expression levels of the L protein as shown in FIGS. 25A-25C, on days 2 and 5 post transfection. The L protein significantly increased saRNA mediated expression of NanoLuciferase.

FIGS. 27A-27C show the effect of single constructs encoding both the L protein and the luciferase gene, in which the mRNA contained a pro-inflammatory ARCA cap and a 50%-50% mixture of natural uridines and pseudo-uridines, 48 hours after transfection in human fibroblasts (FIG. 27A), A549 cells (FIG. 27B), and C2C12 mouse muscle cells (FIG. 27C).

FIGS. 28A-28C show mRNA expression of the L protein virtually eliminated the induction of IFN-beta expression by the ARCA-capped and 50% natural uridine-containing mRNA in human fibroblasts (FIG. 28A), A549 cells (FIG. 28B), and C2C12 mouse muscle cells (FIG. 28C), as measured by IFN-beta ELISA.

FIGS. 29A-29D show that in the experiment described in FIGS. 27A-27C and FIGS. 28A-28D, the induction of other proinflammatory modulators: IFN beta FIG. 29A; IFN lambda1 FIG. 29B; IFN lambda 2/3 FIG. 29C; and IP-10 FIG. 29D; were also eliminated mRNA expression of the L protein.

FIGS. 30A-30H show example configurations of self-amplifying RNA constructs encoding non-structural proteins (NSP1, NSP2, NSP3, and NSP4) subgenomic promoters (SGP), the gene of interest (GOI), internal ribosome entry sites (IRES), the P2A self-cleaving site, the L protein (EG tech) and 5′ and 3′ UTRs, as well as RNA caps and poly-adenosine tails. The saRNA constructs provided herein may be designed as cis-replicons in which the gene of interest (GOI), L protein, and saRNA replicase cassettes are part of the same sequence of the saRNA (FIGS. 30A-30C), as trans-replicons in which at least one RNA molecule containing the GOI and/or L protein does not contain the saRNA replicase cassette and the saRNA replicase cassette is provided on a different saRNA molecule (FIG. 30D-30F), or, as a partial saRNA replicon combination in which at least one of the saRNA components such as a component of the replicase cassette GOI and/or the L protein is provided as a replication incompetent RNA, e.g., as a messenger RNA (FIGS. 30G-30H).

### DETAILED DESCRIPTION

Disclosed herein are various compositions, methods, and kits for improving target protein expression. In one embodiment, the compositions, methods, and kits of the disclosure provide ribonucleic acids (RNA) encoding a nucleocytoplasmic transport (NCT) inhibitor protein to improve target protein expression. As provided herein, an RNA encoding an NCT inhibitor protein may improve the duration, quality, and/or quantity of expression of a target protein encoded by an RNA or DNA that is introduced into a cell. In some embodiments, the NCT inhibitor protein improves self-amplifying RNA (saRNA) expression of a target protein introduced into a cell. In some embodiments, an RNA encoding an NCT inhibitor protein improves the expression of a target protein RNA containing at least one unmodified uridine nucleotide introduced into a cell.

In some embodiments of the disclosure, the target protein is encoded by a DNA and the NCT inhibitor protein is encoded by an RNA, e.g., a messenger RNA. In some embodiments, the target protein is encoded by an RNA, e.g., a messenger RNA and the NCT inhibitor protein is encoded by the same or a separate messenger RNA. In some embodiments, the target protein is encoded by a self-amplifying RNA (saRNA) and the NCT inhibitor protein is encoded by the same or a separate saRNA, or by an mRNA. As provided herein, an RNA encoding an NCT inhibitor protein may improve the duration, quality, and/or quantity of expression of a target protein encoded by DNA or RNA, e.g., an saRNA or an mRNA which is introduced into a cell.

In some embodiments, an mRNA or saRNA of the disclosure encoding a target protein comprises at least one unmodified uridine nucleotide. As provided herein, introduction of an RNA encoding an NCT inhibitor protein may improve the duration, quality, and/or quantity of expression of a target protein encoded by an RNA comprising at one unmodified uridine nucleotide which is introduced into the cell.

The combination of a target protein encoded by an RNA, and an NCT inhibitor protein encoded by a DNA is also provided herein.

In some embodiments, the nucleocytoplasmic transport (NCT) inhibitor may be selected from the group consisting of a picornavirus leader (L) protein, a picornavirus 2A protease, a rhinovirus 3C protease, a herpes simplex virus (HSV) ICP27 protein, and a rhabdovirus matrix (M) protein.

As discussed herein throughout, RNA mediated inhibition of nucleocytoplasmic transport by an NCT inhibitor protein may inhibit cell signaling, e.g., interferon-beta, in response to cell stress, and may improve the cell response to innate immune signaling; pro-inflammatory signaling; delivery vector induced inflammation; natural nucleoside mRNA transfection; self-amplifying RNA transfection; and expression of a target protein. Without being bound by theory, it is thought that the compositions and methods of the disclosure prevent regulatory mechanisms of the cell from activating in response to expression of a target protein, and that this improves yields and/or functionality of the target protein.

In some embodiments, the methods and systems of the disclosure, e.g., introduction of an RNA encoding an NCT inhibitor, are shown to improve regulation of the cellular stress response and prevent disadvantageous effects on target protein expression caused by induction of inflammation and interferon in vivo associated with the introduction of DNA and RNA into a cell.

### I. Definitions

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

It must be noted that, as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “native” or “wild-type” as used herein refers to a nucleotide sequence, e.g. gene, or gene product, e.g. RNA or polypeptide, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence.

The terms “individual,” “subject,” and “patient” are used interchangeably herein and refer to any subject for whom treatment or therapy is desired. The subject may be a mammalian subject. Mammalian subjects include, e.g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion or service animal (e.g. cats or dogs).

The terms “treating” or “treatment” are used herein to generally mean obtaining a desired pharmacologic and/or physiologic effect with a therapeutic agent. The terms treating or treatment can include the treatment of a cell, a plurality of cells, a tissue, or a subject. The effect may be prophylactic in terms of completely or partially preventing a disease or symptom thereof, e.g. reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein includes any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject therapy will be administered before the symptomatic stage of the disease; and, in some embodiments, during the symptomatic stage of the disease; and, in some embodiments, after the symptomatic stage of the disease.

It is to be understood that this invention is not limited to the particular methodology, products, apparatus and factors described, as such methods, apparatus and formulations may, of course, vary. It is also to be understood that the terminology used herein is for the purpose of describing particular embodiments only, and it is not intended to limit the scope of the present invention which will be limited only by appended claims.

### II. Target Proteins

Provided herein are compositions comprising an RNA encoding a nucleocytoplasmic transport (NCT) inhibitor protein, and a deoxyribonucleic acid (DNA) or ribonucleic acid (RNA) encoding a target protein. Also provided herein are methods for improvement of expression of a target protein with an RNA encoding an NCT inhibitor protein. Examples of target protein sequences that may be introduced into a cell with an RNA encoding an NCT inhibitor protein include, but are not limited to, those of Tables 1 and 2.

In some embodiments, the target protein is an antibody; an antibody-like molecule; a receptor; a monoclonal antibody; antibody parts or fragments; a nanobody; a bi-specific or multi-specific antibody; or a bi-specific or multi-specific antibody-like molecule. In some embodiments, the antibody is adalimumab. In some embodiments, the antibody is Abciximab, Alemtuzumab, Alirocumab, Amivantamab, Atezolizumab, Avelumab, Basiliximab, Belimumab, Benralizumab, Bevacizumab, Bezlotoxumab, Blinatumomab, Brentuximab vedotin, Brodalumab, Brolucizumab, Burosumab, Canakinumab, Caplacizumab, Capromab, Catumaxomab, Cemiplimab, Certolizumab pegol, Cetuximab, Crizanlizumab, Daclizumab, Daratumumab, Denosumab, Dinutuximab, Dupilumab, Durvalumab, Eculizumab, Elotuzumab, Emapalumab, Emicizumab, Enfortumab vedotin, Eptinezumab, Erenumab, Ertumaxomab, Etaracizumab, Evolocumab, Fremanezumab, Galcanezumab, Gemtuzumab ozogamicin, Golimumab, Guselkumab, Ibalizumab, Ibritumomab tiuxetan, Idarucizuma, Imciromab, Infliximab, Inotuzumab ozogamicin, Ipilimumab, Isatuximab, Itolizumab, Ixekizumab, Lanadelumab, Lokivetmab, Mepolizumab, Mogamulizumab, Moxetumomab Pasudotox, Natalizumab, Necitumumab, Nimotuzumab, Nivolumab, Obiltoxaximab, Obinutuzumab, Ocrelizumab, Ofatumumab, Olaratumab, Omalizumab, Palivizumab, Panitumumab, Pembrolizumab, Pertuzumab, Polatuzumab vedotin, Racotumomab, Ramucirumab, Ranibizumab, Raxibacumab, Ravulizumab, Reslizumab, Risankizumab, Rituximab, Rmab, Romosozumab, Rovelizumab, Ruplizumab, Sacituzumab govitecan, Sarilumab, Secukinumab, Siltuximab, Talquetamab, Teclistamab, Teprotumumab, Tildrakizumab, Tocilizumab, Tositumomab, Trastuzumab, Trastuzumab duocarmazine, Trastuzumab emtansine, Ustekinumab, and Vedolizumab. Polypeptide sequences for such antibodies are publicly available—for example, in the Thera-SAbDab database (at opig.stats.ox.ac.uk), described in Raybould et al. (2020) Thera-SAbDab: the Therapeutic Structural Antibody Database. Nucleic Acids Res. 48 (D1): gkz827.

In some embodiments, the target protein is a bi-specific or multi-specific antibody; or a bi-specific or multi-specific antibody-like molecule. In some embodiments, the bispecific antibody is Blinatumomab and Emicizumab. In some embodiments, the target protein is a bi-specific T-cell engager (BiTE), such as, for example, Blinatumomab (MT103) and Solitomab. In some embodiments, the target protein is a binding ligand or binder based on protein scaffold (such as, adnectin, anticalin, avimer, fynomer, Kunitz domain, Knottin, Affibody or DARPin).

In some embodiments, the target protein is a blood protein. Non-limiting examples of a blood protein include transferrin, t-PA, hirudin, C1 esterase inhibitor, anti-thrombin, plasma kallikrein inhibitor, plasmin, pro-thrombin complex, complement components, Prealbumin (transthyretin), Alpha 1 antitrypsin, Alpha-1-acid glycoprotein, Alpha-1-fetoprotein, alpha2-macroglobulin, Gamma globulins, Beta-2 microglobulin, Haptoglobin, Ceruloplasmin, Complement component 3, Complement component 4, C-reactive protein (CRP), Lipoproteins (chylomicrons, very low density lipoprotein (VLDL), low density lipoprotein (LDL), high density lipoprotein (HDL)), Transferrin, Prothrombin, mannose binding lectin (MBL), albumins, globulins, fibrinogen, regulatory factors, and coagulation factors, such as, Factor I, Factor II, Factor III, Factor IV, Factor V, Factor VI, Factor VII, Factor IX, Factor X, Factor XI, Factor XII, Factor XIII, von Willeband factor, prekallikrein, Fitzgerald factor, fibronectin, anti-thrombin III, heparin cofactor II, protein C, protein S, protein Z, protein Z-related protease inhibitor, plasminogen, alpha 2-antiplasmin, tissue plasminogen activator, urokinase, plasminogen activator inhibitor-1, plasminogen activator inhibitor-2, and cancer procoagulant. In some embodiments, the target protein is a thrombolytic. Non-limiting examples of thrombolytics include Eminase (anistreplase), Retavase (reteplase), Streptase (streptokinase, kabikinase), alteplase, t-PA (class of drugs that includes Activase), TNKase (tenecteplase), Abbokinase, and Kinlytic (rokinase).

In some embodiments, the target protein is a growth factor. Non-limiting examples of growth factors include erythropoietin (EPO), Insulin like growth factor-1 (IGF-1), Granulocyte colony-stimulating factor (G-CSF), Granulocyte-macrophage colony-stimulating factor (GM-GCF), Bone morphogenetic protein-2 (BMP-2), Bone morphogenetic protein-7 (BMP-7), keratinocyte growth factor (KGF), Platelet-derived growth factor (PDGF), Adrenomedullin (AM), Angiopoictin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Colony-stimulating factors, Macrophage colony-stimulating factor (M-CSF), Epidermal growth factor (EGF), Ephrins-Ephrin A1, Ephrin A2, Ephrin A3, Ephrin A4, Ephrin A5, Ephrin B1, Ephrin B2, Ephrin B3, each of Fibroblast growth factor (FGF) 1, FGF2, FGF3, FGF4, FGF5, FGF6, FGF7, FGF8, FGF9, FGF10, FGF 11, FGF12, FGF13, FGF14, FGF15, FGF16, FGF17, FGF18, FGF19, FGF20, FGF21, FGF22, FGF23, Foetal Bovine Somatotrophin (FBS), GDNF family of ligands, Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factors, Insulin-like growth factor-1 (IGF-1), Insulin-like growth factor-2 (IGF-2), Interleukin-1 (IL-1), IL-2, IL-3, IL-4, IL-5, IL-6, IL-7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1) Neuregulin 2 (NRG2), Neuregulin 3 (NRG3), Neuregulin 4 (NRG4), Neurotrophins, Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS), T-cell growth factor (TCGF), Thrombopoietin (TPO), Transforming growth factor alpha (TGF-α), Transforming growth factor beta (TGF-β), Vascular endothelial growth factor (VEGF), and Wnt Signaling Pathway. In some embodiments, the target protein is a hormone. Non-limiting examples of hormones include glucagon like peptide-1, insulin, human growth hormone, follicle stimulating hormone, calcitonin, lutropin, glucagon like peptide-2, leptin, parathyroid hormone, chorionic gonadotropin, thyroid stimulating hormone, erythropoietin, and glucagon.

In some embodiments, the target protein is an enzyme. Non-limiting examples of an enzyme include Alpha-glycosidase, glucocerebrosidase, iduronate-2-sulfate, alpha-galactosidase, urate oxidase, N-acetyl-galactosidase, carboxypeptidase, hyaluronidase, DNAse, asparaginasc, uricase, adenosine deaminase and other enterokinases, cyclases, caspases, cathepsins, oxidoreductases, transferases, hydrolases, lyases, isomerases, and ligases. A target protein for expression through the use of the present compositions and methods may include proteins related to enzyme replacement, such as Agalsidase beta, Agalsidase alfa, Imiglucerase, Taligulcerase alfa, Velaglucerase alfa, Alglucerase, Sebelipase alpha, Laronidase, Idursulfase, Elosulfase alpha, Galsulfase, Alglucosidase alpha, C3 inhibitor, Glucose-6-phosphatase catalytic subunit 1, Phenylalanine hydroxylase, Hurler and Hunter corrective factors. In some embodiments, the present compositions and methods are used for enzyme production. Such enzymes may be useful in the production of clinical testing kits or other diagnostic assays.

In some embodiments, the target protein is a membrane protein. Illustrative membrane proteins include ion channels, gap junctions, ionotropic receptors, transporters, integral membrane proteins such as cell surface receptors, proteins that shuttle between the membrane and cytosol in response to signaling, and the like. In some embodiments, the cell surface receptor is G-protein coupled receptors (GPCRs), tyrosine kinase receptors, integrins and the like. In some embodiments, the cell surface receptor is a G protein-coupled receptor. In some embodiments, the target protein is a seven-(pass)-transmembrane domain receptor, 7-transmembrane (7-TM) receptor, heptahelical receptor, serpentine receptor, or G protein-linked receptor (GPLR). In some embodiments, the target protein is a Class A GPCR, Class B GPCR, Class C GPCR, Class D GPCR, Class E GPCR, or Class F GPCR. In some embodiments, the target protein is a Class 1 GPCR, Class 2 GPCR, Class 3 GPCR, Class 4 GPCR, Class 5 GPCR, or Class 6 GPCR. In some embodiments, the target protein is a Rhodopsin-like GPCR, a Secretin receptor family GPCR, a Metabotropic glutamate/pheromone GPCR, a Fungal mating pheromone receptor, a Cyclic AMP receptor, or a Frizzled/Smoothened GPCR. In some embodiments, the cell surface receptor is IL-1 receptor, IL-1Ra, tumor necrosis factor receptor (TNFR), or vascular endothelial growth factor receptor (VEGFR). In some embodiments, the target protein is a receptor mimic. In some embodiments, the target protein is a protein that shuttles between the membrane and cytosol in response to signaling, such as, Ras protein, Rac protein, Raf protein, Ga subunits, arrestin, Src protein and other effector proteins.

In some embodiments, the target protein is a nucleosidase, an NAD+ nucleosidase, a hydrolase, a glycosylase, a glycosylasc that hydrolyzes N-glycosyl compounds, an NAD+ glycohydrolase, an NADase, a DPNase, a DPN hydrolase, an NAD hydrolase, a diphosphopyridine nucleosidase, a nicotinamide adenine dinucleotide nucleosidase, an NAD glycohydrolase, an NAD nucleosidase, or a nicotinamide adenine dinucleotide glycohydrolase. In some embodiments, the target protein is an enzyme that participates in nicotinate and nicotinamide metabolism and calcium signaling pathway.

In some embodiments, the target protein is selected from the group consisting of Abatacept, Aflibercept, Agalsidase beta, Albiglutide, Aldesleukin, Alefacept, Alglucerase, Alglucosidase alfa, Aliskiren, Alpha-1-proteinase inhibitor, Alteplase, Anakinra, Ancestim, Anistreplase, Anthrax immune globulin human, Antihemophilic Factor, Antithrombin Alfa, Antithrombin III human, Antithymocyte globulin, Anti-thymocyte Globulin (Equine), Anti-thymocyte Globulin (Rabbit), Aprotinin, Arcitumomab, Asfotase Alfa, Asparaginase, Asparaginase Erwinia chrysanthemi, Becaplermin, Belatacept, Beractant, Bivalirudin, Botulinum Toxin Type A, Botulinum Toxin Type B, Buscrelin, C1 Esterase Inhibitor (Human), C1 Esterase Inhibitor, Choriogonadotropin alfa, Chorionic Gonadotropin (Human), Chorionic Gonadotropin, Coagulation factor IX, Coagulation factor VIIa, Coagulation factor X human, Coagulation Factor XIII A-Subunit, Collagenase, Conestat alfa, Corticotropin, Cosyntropin, Daptomycin, Darbepoetin alfa, Defibrotide, Denileukin diftitox, Desirudin, Dornase alfa, Drotrecogin alfa, Dulaglutide, Efalizumab, Efmoroctocog alfa, Elosulfase alfa, Enfuvirtide, Epoetin alfa, Epoetin zeta, Eptifibatide, Etanercept, Exenatide, Factor IX Complex (Human), Fibrinogen Concentrate (Human), Fibrinolysin aka plasmin, Filgrastim, Filgrastim-sndz, Follitropin alpha, Follitropin beta, Galsulfase, Gastric intrinsic factor, Glatiramer acetate, Glucagon recombinant, Glucarpidase, Gramicidin D, Hepatitis A Vaccine, Hepatitis B immune globulin, Human calcitonin, Human Clostridium tetani toxoid immune globulin, Human rabies virus immune globulin, Human Rho (D) immuno globulin, Human Serum Albumin, Human Varicella-Zoster Immune Globulin, Hyaluronidase, Hyaluronidase, Ibritumomab, Idursulfase, Imiglucerase, Immune Globulin Human, Infliximab, Insulin aspart, Insulin Beef, Insulin Degludec, Insulin detemir, Insulin Glargine, Insulin glulisine, Insulin Lispro, Insulin Pork, Insulin Regular, Insulin Regular, Insulin, porcine, Insulin, isophane, Interferon Alfa-2a, Recombinant, Interferon alfa-2b, Interferon alfacon-1, Interferon alfa-n1, Interferon alfa-n9, Interferon beta-1a, Interferon beta-1b, Interferon gamma-1b, Intravenous Immunoglobulin, Ipilimumab, Ixekizumab, Laronidase, Lenograstim, Lepirudin, Leuprolide, Liraglutide, Lucinactant, Lutropin alfa, Lutropin alfa, Mecasermin, Menotropins, Epoetin beta, Metreleptin, Muromonab, alpha interferon, Nesiritide, Ocriplasmin, Omalizumab, Oprelvekin, OspA lipoprotein, Oxytocin, Palifermin, Pancrelipase, Poractant alfa, Pramlintide, Precotact, Protein S human, Rasburicase, Reteplase, Rilonacept, Rituximab, Romiplostim, Sacrosidase, Salmon Calcitonin, Sargramostim, Satumomab Pendetide, Sebelipase alfa, Secretin, Secukinumab, Sermorelin, Serum albumin, Serum albumin iodonated, Simoctocog Alfa, Sipuleucel-T, Somatotropin Recombinant, Somatropin recombinant, Streptokinase, Sulodexide, Susoctocog alfa, Taliglucerase alfa, Teduglutide, Teicoplanin, Tenecteplase, Teriparatide, Tesamorelin, Thrombomodulin alfa, Thymalfasin, Thyroglobulin, Thyrotropin Alfa, Thyrotropin Alfa, Tocilizumab, Tositumomab, Tuberculin Purified Protein Derivative, Turoctocog alfa, Urofollitropin, Urokinase, Vasopressin, and Velaglucerase alfa.

In some embodiments, the target protein is a biosimilar. In some embodiments, the target protein is a therapeutic polypeptide, such as, a biopharmaceutical drug also known as biologics; a biomarker-enabling polypeptides, such as, a diagnostic, prognostic, or predictive biomarkers; a prophylactic polypeptide, such as, adjuvants, soluble antigens, subviral particles, virus like particles; an auxiliary polypeptides, such as polypeptides supporting an activity or binding of another molecule or inhibiting another protein-protein interaction; a polypeptide used in research, such as antigens for generating novel monoclonal and polyclonal antibodies in animals, reporter proteins, or tool polypeptides for studying physiological or pathological processes and the effect of drugs on these processes in animal models. In some embodiments, the target protein is a protein that has applications in microscopy and imaging, such as, a fluorescent protein. In some embodiments, the target protein is not a reporter protein, such as, for example, luciferase. In some embodiments, the target protein is a human protein.

In some embodiments, the target protein is an immunomodulator. Non-limiting examples of immunomodulators include cytokines, chemokines, interleukins, interferons. In some embodiments, the target protein is an antigen for use as a vaccine or for research. In some embodiments, the target protein is a structural protein, such as a structural protein that functions in protein complex assembly. In some embodiments, the target protein is an anti-microbial polypeptide; or an anti-viral polypeptide. In some embodiments, the target protein is a tumor suppressor. In some embodiments, the target protein is a transcription factor or a translation factor. In some embodiments, the target protein is a pharmacokinetics modulating protein, a small molecule binding protein, an RNA binding protein, or a protein binding protein.

In some embodiments, the target protein is Dopamine receptor 1 (DRD1), Cystic fibrosis transmembrane conductance regulator (CFTR), C1 esterase inhibitor (C1-Inh), IL2 inducible T cell kinase (ITK), or an NADase. In some embodiments, the target protein is a firefly luciferase.

In some embodiments, the target protein is an antigen of a pathogenic organism, e.g., a virus or bacterium. In some embodiments, the target protein is a bacterial antigen—a protein or glycoprotein antigen. In some embodiments, the target protein is a viral antigen. In some embodiments, the viral antigen is derived from a virus selected from the group consisting of coronavirus, influenza virus, Hepatitis B virus, Human Papilloma virus (HPV), West Nile virus, and Human Immunodeficiency Virus (HIV) virus. In some embodiments, the viral protein is derived from a coronavirus. In some embodiments, the coronavirus is a betacoronavirus. In some embodiments, the betacoronavirus is severe acute respiratory syndrome (SARS) virus. In some embodiments, the SARS virus is a SARS-CoV-2 virus. In some embodiments, the betacoronavirus is Middle East respiratory syndrome (MERS) virus.

### Example Target Protein Sequences

In some embodiments, an RNA encoding an NCT inhibitor protein, e.g., an L protein, improves the expression of a target protein, wherein the target protein is an antibody selected from the group comprising (also see Table 1): Adalimumab, Pembrolizumab, Nivolumab, Trastuzumab, Bevacizumab, Ustekinumab, Ocrelizumab, Secukinumab, Vedolizumab, Ibalizumab, Nirsevimab, Atoltivimab, Maftivimab, Odesivimab, Casirivimab, Imdevimab, and Brolucizumab. In some embodiments, an RNA of the present disclosure encodes one or more sequences of Table 1, or an amino acid sequence with at least 70%, at least 75%, at least 80%, at least 85%, at least 90%, at least 95%, at least 96%, at least 97%, at least 98%, or at least 99% sequence identity thereto.

TABLE 1
Example antibody target proteins

| Antibody target protein | VH SEQ ID NO: | VL SEQ ID NO: |
|---|---|---|
| Adalimumab | 13 | 14 |
| Pembrolizumab | 16 | 17 |
| Nivolumab | 18 | 19 |
| Trastuzumab | 20 | 21 |
| Bevacizumab | 22 | 23 |
| Ustekinumab | 24 | 25 |
| Ocrelizumab | 26 | 27 |
| Secukinumab | 28 | 29 |
| Vedolizumab | 30 | 31 |
| Ibalizumab | 32 | 33 |
| Nirsevimab | 34 | 35 |
| Atoltivimab | 36 | 37 |
| Maftivimab | 38 | 39 |
| Odesivimab | 40 | 41 |
| Casirivimab | 42 | 43 |
| Imdevimab | 44 | 45 |
| Brolucizumab | 46 | 47 |

In some embodiments, introduction of an RNA encoding an NCT inhibitor protein, e.g., an L protein, improves the expression of a target protein, wherein the target protein is a blood protein or immune-oncology protein selected from the group comprising (also see Table 2): rFIX-Fc Coagulation Factor IX, Taliglucerase, Agalsidase beta, Alglucosidase alfa, Laronidase, Idursulfase, HLA Class I alpha chain (mouse K2-D1) & B2m (mouse), Nlrc5 (mouse), NLRC5 (human), scIL-12 (mouse), scIL-12 (human), and HLA Class I alpha chain (human) and B2M (human). In some embodiments, an RNA of the present disclosure encodes one or more sequences of Table 2, or an amino acid sequence with at least 70%, at least 75%, at least 80%, at least 85%, at least 90%, at least 95%, at least 96%, at least 97%, at least 98%, or at least 99% sequence identity thereto.

TABLE 2
Example blood and immuno-oncology target proteins

| | Amino acid |
|---|---|
| Target protein | SEQ ID NO: |
| Glucosylceramidase (GBA) | 48 |
| rFIX-Fc Coagulation Factor IX | 49 |
| Taliglucerase | 50 |
| Agalsidase beta | 51 |
| Laronidase | 52 |
| Idursulfase | 53 |
| HLA Class I alpha chain (mouse K2-D1) | 54 |
| B2m (mouse) | 55 |
| Nlrc5 (mouse) | 56 |
| HLA Class I alpha chain | 57 |
| B2M (Human) | 58 |
| Nlrc5 (human) | 59 |
| scIL-12 (mouse) | 60 |
| scIL-12 (human) | 61 |
| Alglucosidase alfa | 62 |
| B-domain deleted human Factor VIII (BDD FVIII) | 63 |
| Von Willebrand Factor, recombinant | 64 |

In some embodiments, an RNA encoding an NCT inhibitor protein, e.g., an L protein, improves the expression of a target protein, wherein the target protein is an antigen. In some embodiments, the target protein is a bacterial antigen—a protein or glycoprotein antigen. In some embodiments, the target protein is a viral antigen. In some embodiments, the viral antigen protein is a viral antigen protein is derived from a virus selected from the group consisting of coronavirus, influenza virus, Hepatitis B virus, Human Papilloma virus (HPV), West Nile virus, and Human Immunodeficiency Virus (HIV) virus. In some embodiments, the viral protein is derived from a coronavirus. In some embodiments, the coronavirus is a betacoronavirus. In some embodiments, the betacoronavirus is severe acute respiratory syndrome (SARS) virus. In some embodiments, the SARS virus is a SARS-COV-2 virus. In some embodiments, the betacoronavirus is Middle East respiratory syndrome (MERS) virus.

In some embodiments, the viral protein is derived from West Nile virus. In some embodiments, the viral protein is precursor membrane protein (preM), envelope glycoprotein (E), or a combination thereof.

In some embodiments, the viral antigen comprises one or more of the proteins derived from a virus listed below in Table 3. In some embodiments, an RNA of the present disclosure encodes one or more sequences of Table 3, or an amino acid sequence with at least 70%, at least 75%, at least 80%, at least 85%, at least 90%, at least 95%, at least 96%, at least 97%, at least 98%, or at least 99% sequence identity thereto.

TABLE 3
Example Viral antigen proteins

| Virus | Viral protein |
|---|---|
| Artevirus | GP2, GP3, GP4, GP5 |
| Astrovirus | VP25, VP27, VP34 |
| Avian influenza (H5N1) | HA, NA and M1 |
| BFDV | VP1 |
| BTV | VP3 and VP7 |
| Chikungunya Virus | E1, E2, C |
| Ebola | VP40 and glycoprotein |
| Enterovirus 71 | P1 and 3CD |
| GHPV | VP1 and VP2 |
| HBV | sAg (S protein) |
| HBV | sAg (S protein) and VSPalphaS |
| HBV | sAg (M protein) |
| HBV | Core antigen |
| HBV | Surface and core antigens |
| HBV | sAg (S protein), preS1 and preS2 |
| HCV | Core protein, E1 and E2 |
| HDV | HBsAg and L-HDAg |
| HEV | Capsid protein |
| HIV | Pr55gag |
| HIV | Pr160gag-pol |
| HIV | Gag protein |
| HIV | Pr55gag and RT |
| HIV | Pr55gag and ENV |
| HPV11 | L1 protein |
| HPV16 | L1 protein |
| IBDV | VP2 and VP3 |
| Influenza A | M1 and ESAT6-HA |
| Influenza A | HA (H1N1) and M1 (H3N2) |
| Influenza A | HA (H1N1) and M1 (H3N2) |
| Influenza A | HA (H3N2) and M1 (H1N1) |
| Influenza A H1N1 | HA and M1 |
| Influenza A H3N2 | HA and M1 |
| Influenza A | HA, NA, and M1 |
| Influenza A | HA and/or NA |
| Influenza B | HA and/or NA |
| IPCV | Coat protein |
| LASV | GP |
| JC polyomavirus | VP1 |
| Marburg | VP40 and glycoprotein |
| MS2 | Coat protein |
| NDV | HN, F, NP and MP |
| Norovirus | VP1 |
| No | Capsid protein |
| No | VP1 |
| Nv | Capsid protein |
| PhMV | Coat protein |
| PhMV | Coat protein, CPV epitopes |
| | and F protein (CDV) |
| Polyomavirus | VP1 |
| PPV | VP2 |
| RHDV | VP60 |
| Rhinovirus | Capsid |
| RSV | F, M, and P |
| RSV | F, M, G |
| Rotavirus | VP2, VP6 and VP7 |
| Rotavirus | VP2 and VP6 |
| SARS | SP, EP and MP |
| SIV | Pr55gag and envelope protein |
| SV40 | VP1 |
| SVDV | P1 and 3CD |
| WNV | prM-E |
| Zika virus | prM-E |

### III. Nuclear Cytoplasmic Transport (NCT) Inhibitor Proteins

Provided herein are compositions comprising an RNA encoding a nucleocytoplasmic transport (NCT) inhibitor protein and a deoxyribonucleic acid (DNA) or ribonucleic acid (RNA) encoding a target protein. Also provided herein are methods for improvement of expression of the target protein with an RNA encoding an NCT inhibitor protein. Examples of NCT inhibitor proteins, e.g., L proteins, include but are not limited to, those of Table 4, or an amino acid sequence with at least 70%, at least 75%, at least 80%, at least 85%, at least 90%, at least 95%, at least 96%, at least 97%, at least 98%, or at least 99% sequence identity thereto.

In some embodiments, the NCT inhibitor protein is a nuclear pore blocking viral protein. In some embodiments, the NCT inhibitor protein is a native or synthetic peptide that is capable of blocking the nuclear pore, thereby inhibiting nucleocytoplasmic transport (“NCT”). In some embodiments, the NCT inhibitor protein is a viral protein. In some embodiments, the NCT inhibitor protein is selected from the group consisting of a picornavirus leader (L) protein, a picornavirus 2A protease, a rhinovirus 3C protease, a coronavirus ORF6 protein, an ebolavirus VP24 protein, a Venezuelan equine encephalitis virus (VEEV) capsid protein, a herpes simplex virus (HSV) ICP27 protein, and a rhabdovirus matrix (M) protein.

The NCT inhibitor protein may be a functional variant of any of the proteins disclosed herein. As used herein, the term “functional variant” refers to a protein that is homologous to an original protein and/or shares substantial sequence similarity to that original protein (e.g., an amino acid sequence with at least 70%, at least 75%, at least 80%, at least 85%, at least 90%, at least 95%, at least 96%, at least 97%, at least 98%, or at least 99% sequence identity thereto), and shares one or more functional characteristics of the original protein. For example, a functional variant of an NCT inhibitor protein retains the ability to inhibit NCT.

In some embodiments, the NCT inhibitor protein is a leader (L) protein from a picornavirus or a functional variant thereof. In some embodiments, the NCT inhibitor protein is a leader protein from the Cardiovirus, Hepatovirus, or Aphthovirus genera. For example, the NCT inhibitor protein may be from Bovine rhinitis A virus, Bovine rhinitis B virus, Equine rhinitis A virus, Foot-and-mouth disease virus, Hepatovirus A, Hepatovirus B, Marmota himalayana hepatovirus, Phopivirus, Cardiovirus A, Cardiovirus B, Theiler's Murine encephalomyelitis virus (TMEV), Vilyuisk human encephalomyelitis virus (VHEV), Theiler-like rat virus (TRV), or Saffold virus (SAF-V).

In some embodiments, the NCT inhibitor protein is the L protein of Theiler's virus or a functional variant thereof. In some embodiments, the L protein comprises SEQ ID NO: 1. In some embodiments, the NCT inhibitor protein may comprise an amino acid sequence with at least 70%, at least 75%, at least 80%, at least 85%, at least 90%, at least 95%, at least 96%, at least 97%, at least 98%, or at least 99% sequence identity to SEQ ID NO: 1.

In some embodiments, the L protein is the L protein of Encephalomyocarditis virus (EMCV) or a functional variant thereof. In some embodiments, the L protein comprises SEQ ID NO: 2, or an amino acid sequence with at least 70%, at least 75%, at least 80%, at least 85%, at least 90%, at least 95%, at least 96%, at least 97%, at least 98%, or at least 99% sequence identity thereto.

In some embodiments, the L protein is selected from the group consisting of the L protein of poliovirus, the L protein of HRV16, the L protein of mengo virus, and the L protein of Saffold virus 2 or a functional variant thereof.

In some embodiments, the NCT inhibitor protein is a picornavirus 2A protease or a functional variant thereof. In some embodiments, the NCT inhibitor protein is a 2A protease from Enterovirus, Rhinovirus, Aphtovirus, or Cardiovirus.

In some embodiments, the NCT inhibitor protein is a rhinovirus 3C protease or a functional variant thereof. In some embodiments, the NCT inhibitor protein is a picornavirus 3C protease. In some embodiments, the NCT inhibitor protein is a 3C protease from enterovirus, rhinovirus, aphtovirus, or cardiovirus. For example, in some non-limiting embodiments, the NCT inhibitor protein is a 3C protease from Poliovirus, Coxsackievirus, Rhinovirus, Foot-and-mouth disease virus, or Hepatovirus A.

In some embodiments, the NCT inhibitor protein is a coronavirus ORF6 protein or a functional variant thereof. In some embodiments, the NCT inhibitor protein is a viral protein that disrupts nuclear import complex formation and/or disrupts STAT1 transport into the nucleus.

In some embodiments, the NCT inhibitor protein is an ebolavirus VP24 protein or a functional variant thereof. In some embodiments, the NCT inhibitor protein is an ebolavirus VP40 protein or VP35 protein. In some embodiments, the NCT inhibitor protein is a viral protein that binds to the importin protein karyopherin-α (KPNA). In some embodiments, the NCT inhibitor protein is a viral protein that inhibits the binding of STAT1 to KPNA.

In some embodiments, the NCT inhibitor protein is a Venezuelan equine encephalitis virus (VEEV) capsid protein or a functional variant thereof. In some embodiments, the NCT inhibitor protein is a viral capsid protein that interacts with the nuclear pore complex.

In some embodiments, the NCT inhibitor protein is a herpes simplex virus (HSV) ICP27 protein or a functional variant thereof. In some embodiments, the NCT inhibitor protein is an HSV ORF57 protein.

In some embodiments, the NCT inhibitor protein is a rhabdovirus matrix (M) protein or a functional variant thereof. In some embodiments, the NCT inhibitor protein is an M protein from Cytorhabdovirus, Dichorhavirus, Ephemerovirus, Lyssavirus, Novirhabdovirus, Nucleorhabdovirus, Perhabdovirus, Sigmavirus, Sprivivirus, Tibrovirus, Tupavirus, Varicosavirus, or Vesiculovirus.

In some embodiments, an NCT inhibitor protein may have an amino acid sequence comprising one or more of SEQ ID NO: 1, 2, 3, 4, 5, 6, 7, 8, 9, 10, or 11, or an amino acid sequence with at least 70%, at least 75%, at least 80%, at least 85%, at least 90%, at least 95%, at least 96%, at least 97%, at least 98%, or at least 99% sequence identity thereto.

TABLE 4
Example NCT inhibitor proteins
pore

| Nuclear pore | | | |
|---|---|---|---|
| blocking viral | | | |
| protein | Origin | Family | Amino acid sequence |
| Leader protein | Theiler's virus | Picornaviridae | MACKHGYPDVCPICTAVDATPGFE |
| | | | YLLMADGEWYPTDLLCVDLDDDV |
| | | | FWPSDTSNQSQTMDWTDVPLIRDIV |
| | | | MEPQ |
| | | | (SEQ ID NO: 12) |
| Leader protein | Theiler's-like | Picornaviridae | MACKHGYPLMCPLCTALDKTSDGL |
| | cardiovirus | | FTLLFDNEWYPTDLLTVDLEDEVFY |
| | | | PDDPHMEWTDLPLIQDIEMEPQ |
| | | | (SEQ ID NO: 1) |
| Leader protein | EMCV | Picornaviridae | MATTMEQETCAHSLTFEECPKCSA |
| | | | LQYRNGFYLLKYDEEWYPEELLTD |
| | | | GEDDVFDPELDMEVVFELQ |
| | | | (SEQ ID NO: 2) |
| Leader protein | Poliovirus | Picornaviridae | NYHLATQDDLQNAVNVMWSRDLL |
| | (Enterovirus C) | | VTESRAQGTDSIARCNCNAGVYYC |
| | | | ESRRKYYPVSFVGPTFQYMEANNY |
| | | | YPARYQSHMLIGHGFASPGDCGGIL |
| | | | RCHHGVIGIITAGGEGLVAFSDIRDL |
| | | | YAYEE |
| | | | (SEQ ID NO: 3) |
| Leader protein | Equine rhinitis B | Picornaviridae | MVTMAGNMICNVFAGLATEICSPK |
| | virus 1 | | QGPLLDNELPLPLELAEFPNKDNNC |
| | | | WVAALSHYYTLCDVTNHVTKVTPT |
| | | | TSGIRYYLTAWQSILQTDLENGYYP |
| | | | AAFAVETGLCHGPFPMQQHGYVRN |
| | | | ATSHPYNFCLCSEPVPGEDYWHAV |
| | | | VKVDLSRTEARVDKWLCIDDDRM |
| | | | YLSGPPTRVKLASSYKIPTWIESLAQ |
| | | | FCLQLHPVQHRRTLANSLRNEQCR |
| | | | (SEQ ID NO: 4) |
| Leader protein | Mengo virus | Picornaviridae | MATTMEQEICAHSMTFEECPKCSA |
| | (Cardiovirus) | | LQYRNGFYLLKYDEEWYPEESLTD |
| | | | GEDDVFDPDLDMEVVFETQ |
| | | | (SEQ ID NO: 5) |
| Leader protein | Saffold virus 2 | Picornaviridae | MACKHGYPFLCPLCTAIDTTHDGSF |
| | (Cardiovirus) | | TLLIDNEWYPTDLLTVDLDDDVFHP |
| | | | DDSVMEWTDLPLIQDVVMEPQ |
| | | | (SEQ ID NO: 6) |
| 2A protease | Poliovirus | Picornaviridae | GFGHQNKAVYTAGYKICNYHLATQ |
| | (Enterovirus C) | | DDLQNAVNVMWSRDLLVTESRAQ |
| | | | GTDSIARCNCNAGVYYCESRRKYY |
| | | | PVSFVGPTFQYMEANNYYPARYQS |
| | | | HMLIGHGFASPGDCGGILRCHHGVI |
| | | | GIITAGGEGLVAFSDIRDLYAYEEE |
| | | | AMEQ |
| | | | (SEQ ID NO: 7) |
| 3C protease | HRV16 | Picornaviridae | GPEEEFGMSIIKNNTCVVTTTNGKF |
| | | | TGLGIYDRILILPTHADPGSEIQVNGI |
| | | | HTKVLDSYDLFNKEGVKLEITVLKL |
| | | | DRNEKFRDIRKYIPESEDDYPECNL |
| | | | ALVANQTEPTIIKVGDVVSYGNILL |
| | | | SGTQTARMLKYNYPTKSGYCGGVL |
| | | | YKIGQILGIHVGGNGRDGFSSMLLR |
| | | | SYFTEQ |
| | | | (SEQ ID NO: 8) |
| M protein | Vesicular stomatitis | Rhabdoviridae | MSSLKKILGLKGKGKKSKKLGIAPP |
| | virus | | PYEEDTSMEYAPSAPIDKSYFGVDE |
| | | | MDTYDPNQLRYEKFFFTVKMTVRS |
| | | | NRPFRTYSDVAAAVSHWDHMYIG |
| | | | MAGKRPFYKILAFLGSSNLKATPAV |
| | | | LADQGQPEYHTHCEGRAYLPHRM |
| | | | GKTPPMLNVPEHFRRPFNIGLYKGT |
| | | | IELTMTIYDDESLEAAPMIWDHENS |
| | | | SKFSDFREKALMFGLIVEKKASGA |
| | | | WVLDSISHFK |
| | | | (SEQ ID NO: 9) |
| Non-structural | Influenza A virus | Orthomyxoviridae | MDPNTVSSFQVDCFLWHVRKRVA |
| Protein 1 | | | DQELGDAPFLDRLRRDQKSLRGRG |
| | | | STLGLDIETATRAGKQIVERILKEES |
| | | | DEALKMTMASVPASRYLTDMTLEE |
| | | | MSRDWSMLIPKQKVAGPLCIRMDQ |
| | | | AIMDKNIILKANFSVIFDRLETLILLR |
| | | | AFTEEGAIVGEISPLPSLPGHTAEDV |
| | | | KNAVGVLIGGLEWNDNTVRVSETL |
| | | | QRFAWRSSNENGRPPLTPKQKREM |
| | | | AGTIRSEV |
| | | | (SEQ ID NO: 10) |
| Immediate-early | Simplexvirus | Herpesviridae | MATDIDMLIDLGLDLSDSDLDEDPP |
| protein IE63 | | | EPAESRRDDLESDSSGECSSSDEDM |
| | | | EDPHGEDGPEPILDAARPAVRPSRP |
| | | | EDPGVPSTQTPRPTERQGPNDPQPA |
| | | | PHSVWSRLGARRPSCSPEQHGGKV |
| | | | ARLQPPPTKAQPARGGRRGRRRGR |
| | | | GRGGPGAADGLSDPRRRAPRTNRN |
| | | | PGGPRPGAGWTDGPGAPHGEAWR |
| | | | GSEQPDPPGGQRTRGVRQAPPPLM |
| | | | TLAIAPPPADPRAPAPERKAPAADTI |
| | | | DATTRLVLRSISERAAVDRISESFGR |
| | | | SAQVMHDPFGGQPFPAANSPWAPV |
| | | | LAGQGGPFDAETRRVSWETLVAHG |
| | | | PSLYRTFAGNPRAASTAKAMRDCV |
| | | | LRQENFIEALASADETLAWCKMCIH |
| | | | HNLPLRPQDPIIGTTAAVLDNLATR |
| | | | LRPFLQCYLKARGLCGLDELCSRRR |
| | | | LADIKDIASFVFVILARLANRVERG |
| | | | VAEIDYATLGVGVGEKMHFYLPGA |
| | | | CMAGLIEILDTHRQECSSRVCELTA |
| | | | SHIVAPPYVHGKYFYCNSLF |
| | | | (SEQ ID NO: 11) |

In some embodiments, an NCT inhibitor, e.g., an L protein, comprises a sequence of SEQ ID NO: 161, a mutant sequence of any one of 163, 164, 166, or 168, or a sequence with about 70%, about 80%, about 90%, about 91%, about 92%, about 93%, about 94%, about 95%, about 96%, about 97%, about 98%, or about 99% sequence identity thereto.

In some embodiments, expression of an NCT inhibitor protein increases target protein expression level by about 2-fold, about 3-fold, about 4-fold, about 5-fold, about 6-fold, about 7-fold, about 8-fold, about 9-fold, about 10-fold, about 20-fold, about 30-fold, about 40-fold, 50-fold, about 60-fold, about 70-fold, about 80-fold, about 90-fold, about 100-fold, about 200-fold, about 500 fold, or about 1000-fold.

In some embodiments, expression of an NCT inhibitor protein reduces the expression level of a target protein by about 10%, about 20%, about 30%, about 40%, about 50%, about 60%, about 70%, about 80%, or about 90%.

In some embodiments, expression of an NCT inhibitor protein increases the activity of a target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments, expression of an NCT inhibitor protein reduces the EC₅₀ of a target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments, an NCT inhibitor protein and a target protein are expressed in a cell or subject.

In some embodiments, expression of an NCT inhibitor protein increases the duration of time in which a target protein is found in the cell or subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20-fold.

In some embodiments, expression of an NCT inhibitor protein decreases the coefficient of variation (CV %) of the target protein in the tissue of the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

In some embodiments, expression of an NCT inhibitor protein reduces cell signaling in response to a stressor. In some embodiments, the cell signaling in response to a stressor is expression of interferon-beta. In some embodiments, introducing the NCT inhibitor reduces interferon-beta expression in the cell by about 10-fold, about 100-fold, about 1000-fold, about 10,000 fold, or about 100,000 fold.

In some embodiments, the NCT inhibitor protein improves target protein expression characteristics including but not limited to: yield, quality, folding, posttranslational modification, activity, localization, and downstream activity. In some embodiments, the NCT inhibitor protein may reduce one or more of misfolding, altered activity, incorrect posttranslational modifications, and/or toxicity. In some embodiments, expression of the NCT inhibitor protein increases the duration, quality and/or quantity of the target protein.

In some embodiments, an RNA encoding an NCT inhibitor protein lessens the interferon sensitivity and inflammation associated with target protein expression.

It is also contemplated therein than antibodies to NCT proteins and short interfering RNA (siRNA) of NCT proteins may improve target protein expression. For example, an mRNA encoding for anti-Xpo7 nanobody, an mRNA encoding for anti-NFT2 antibody, an mRNA encoding for Ran Q69L mutant protein, an mRNA encoding for MxB K131A mutant protein, an mRNA encoding a myristoylated ERK-derived phosphomimetic peptide, an mRNA encoding myristoylated SMAD-derived phosphomimetic peptide, an siRNA targeted to Ran mRNA, an siRNA targeted to NTF2 mRNA, an siRNA targeted to MxB mRNA, an siRNA targeted to Xpo7 mRNA, or an siRNA targeted to Nup93 mRNA may be used. NCT inhibitory siRNAs, for example, are commercially obtained as chemically synthesized sequences (IDT Technologies).

### IV. RNA Compositions

Provided herein are compositions comprising an RNA encoding a nucleocytoplasmic transport (NCT) inhibitor protein and a deoxyribonucleic acid (DNA) or ribonucleic acid (RNA) encoding a target protein. In some embodiments, an RNA encoding an NCT inhibitor protein and an RNA encoding a target protein are messenger RNA. In some embodiments, an RNA encoding an NCT inhibitor protein and an RNA encoding a target protein are self-amplifying RNA. In some embodiments, an RNA encoding an NCT inhibitor protein is a messenger RNA and an RNA encoding a target protein is a self-amplifying RNA. In some embodiments, an RNA encoding an NCT inhibitor protein is a self-amplifying RNA and an RNA encoding a target protein is a messenger RNA.

In some embodiments, a composition of the disclosure comprises a messenger RNA encoding both the NCT inhibitor protein and the target protein. In some embodiments, a composition comprises a first messenger RNA encoding an NCT inhibitor protein and a second messenger RNA encoding a target protein.

In some embodiments, an RNA of the disclosure, e.g., an saRNA or an mRNA encoding an NCT inhibitor is delivered to a cell at the same time or on the same day as an saRNA, an mRNA, or a DNA encoding a target protein. In some embodiments, an RNA of the disclosure, e.g., an saRNA or an mRNA encoding an NCT inhibitor is delivered to a cell separately or on a different day from an saRNA, an mRNA, or a DNA encoding a target protein.

In some embodiments, an RNA, e.g., an saRNA or an mRNA encoding an NCT inhibitor is delivered to a cell before an saRNA, an mRNA, or a DNA encoding a target protein. In some embodiments, an RNA, e.g., an saRNA or an mRNA encoding an NCT inhibitor is delivered to a cell about 1 hour, about 2 hours, about 3 hours, about 4 hours, about 5 hours, about 10 hours, about 14 hours, about 1 day, about 2 days, about 3 days, about 4 days, about 5 days, about 1 week, about 2 weeks, or about 3 weeks before an saRNA, an mRNA, or a DNA encoding a target protein is delivered to a cell.

In some embodiments, an RNA, e.g., an saRNA or an mRNA encoding an NCT inhibitor is delivered to a cell after an saRNA, an mRNA, or a DNA encoding a target protein. In some embodiments, an RNA, e.g., an saRNA or an mRNA encoding an NCT inhibitor is delivered to a cell about 1 hour, about 2 hours, about 3 hours, about 4 hours, about 5 hours, about 10 hours, about 14 hours, 1 day, about 2 days, about 3 days, about 4 days, or about 5 days, about 1 week, about 2 weeks, or about 3 weeks after an saRNA, an mRNA, or a DNA encoding a target protein is delivered to a cell.

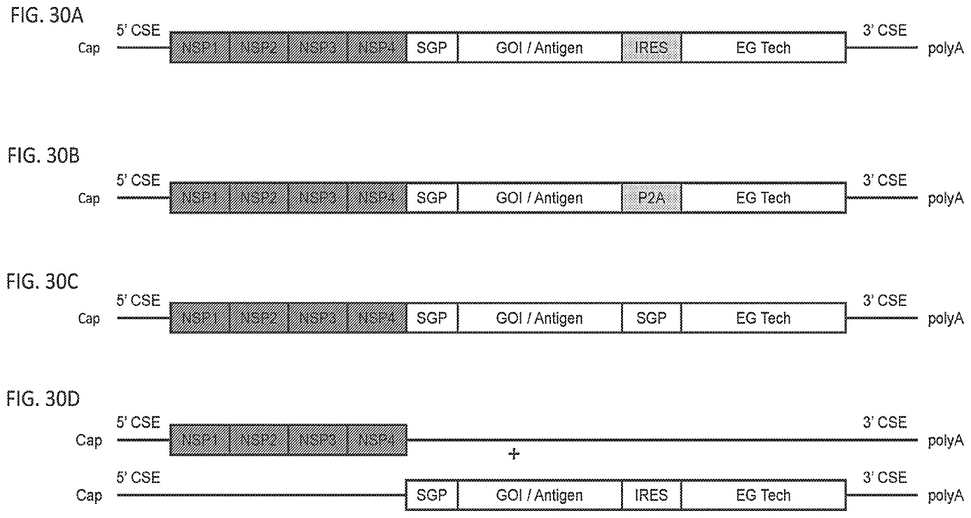

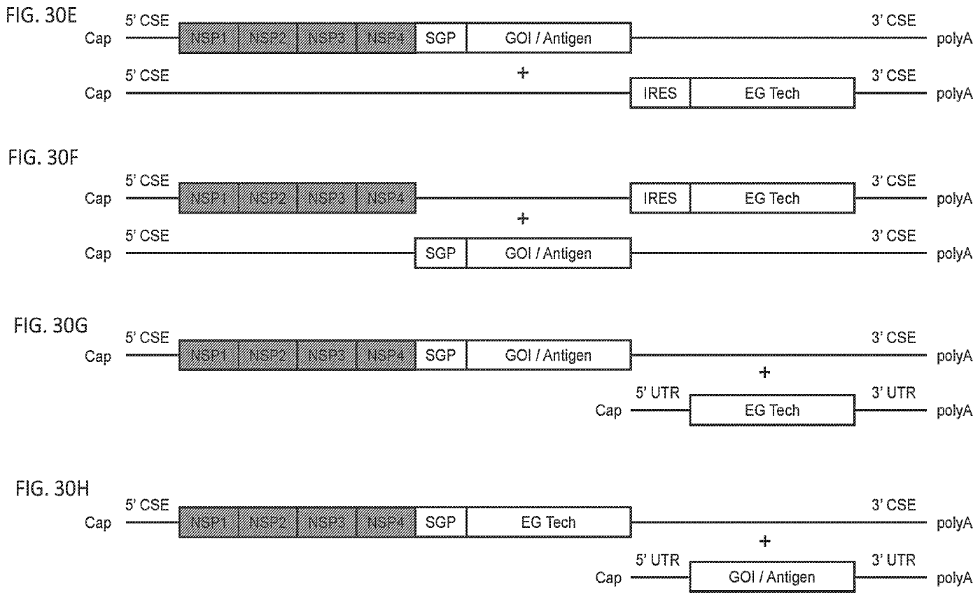

In some embodiments, the L protein and/or the gene of interest (GOI) are expressed using one or more saRNA vectors. Any saRNA vector known in art, including ones based on alphaviruses and flaviviruses, or any novel saRNA vectors may be used. Non-exhaustive examples include saRNA vectors derived from Venezuelan equine encephalitis virus (VEEV), Semliki forest virus (SFV), Sindbis virus (SINV), VEE-SINV chimeras, Tick-borne encephalitis virus (TBEV), Classical swine fever virus (CSFV), and others. saRNA expression vectors can be designed as cis-replicons (in which GOI, L protein and saRNA replicase cassettes are part of the same RNA sequence of the saRNA), as trans-replicons (in which at least one RNA molecule containing GOI and/or L protein does not contain the saRNA replicase cassette, whereas the saRNA replicase cassette is provided on a different saRNA molecule), or as partial saRNA replicon combination (in which at least one of the saRNA components, such as a component of the replicase cassette, GOI and/or the L protein, is provided as a replication incompetent RNA, e.g. as a regular mRNA). Illustrations of selected cis-, trans- and partial saRNA replicons are provided in FIGS. 30A-30H.

Example NCT Inhibitor mRNA Sequences

In some embodiments, a messenger RNA (mRNA) encoding a wild type NCT inhibitor, e.g., an L protein, is encoded by SEQ ID NO: 160, or a sequence with at least 70%, at least 80%, at least 90%, at least 95%, at least 96%, at least 97%, at least 98%, or at least 99% sequence identity thereto.

In some embodiments, a messenger RNA (mRNA) encoding a mutant NCT inhibitor, e.g., an L protein with an E39A mutation, is encoded by SEQ ID NO: 162, or a sequence with at least 70%, at least 80%, at least 90%, at least 95%, at least 96%, at least 97%, at least 98%, or at least 99% sequence identity thereto.

In some embodiments, a messenger RNA (mRNA) encoding a mutant NCT inhibitor, e.g., an L protein with a K35Q mutation, is encoded by SEQ ID NO: 164, or a sequence with at least 70%, at least 80%, at least 90%, at least 95%, at least 96%, at least 97%, at least 98%, or at least 99% sequence identity thereto.

In some embodiments, a messenger RNA (mRNA) encoding a mutant NCT inhibitor, e.g., an L protein with a Y36F mutation, is encoded by SEQ ID NO: 166, or a sequence with at least 70%, at least 80%, at least 90%, at least 95%, at least 96%, at least 97%, at least 98%, or at least 99% sequence identity thereto.

In some embodiments, a messenger RNA (mRNA) encoding a mutant NCT inhibitor, e.g., an L protein with a D37A mutation, is encoded by SEQ ID NO: 168, or a sequence with at least 70%, at least 80%, at least 90%, at least 95%, at least 96%, at least 97%, at least 98%, or at least 99% sequence identity thereto.

In some embodiments, a messenger RNA (mRNA) encoding an NCT inhibitor, e.g., an L protein comprises a UTR sequence encoded by a sequence of SEQ ID NO: 174 and/or SEQ ID NO: 175.

In some embodiments, a messenger RNA (mRNA) encoding an NCT inhibitor, e.g., an L protein is encoded by the sequence of SEQ ID NO: 177.

Example Target Protein mRNA Sequences

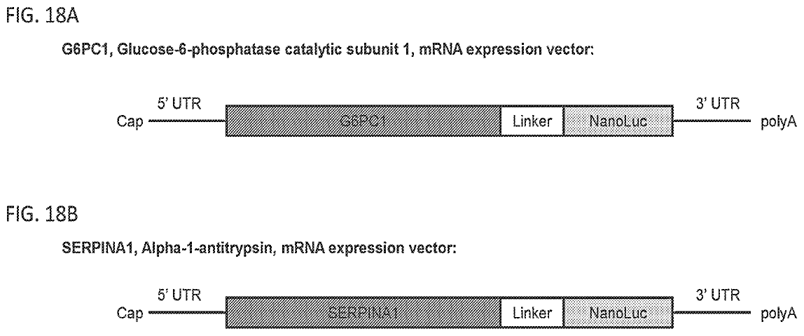

In some embodiments, a messenger RNA (mRNA) of the disclosure is encoded by the following expression vector, in the sequence order represented in FIGS. 18A and 18B, or a sequence with at least 70%, at least 80%, at least 90%, at least 95%, at least 96%, at least 97%, at least 98%, or at least 99% sequence identity thereto. In some embodiments, an mRNA of the disclosure is encoded by the sequences the following expression vector with a different coding sequence, e.g. SERPINA1 (SEQ ID NO: 104) or other coding sequence, or a sequence with at least 70%, at least 80%, at least 90%, at least 95%, at least 96%, at least 97%, at least 98%, or at least 99% sequence identity thereto:
hBG 5′UTR sequence: (SEQ ID NO: 98),
G6PC1 coding sequence: (SEQ ID NO: 99),
Linker sequence: (SEQ ID NO: 100),
NanoLuc reporter coding sequence: (SEQ ID NO: 101), and
hBg 3′UTR sequence: (SEQ ID NO: 102)
   Example saRNA Sequences

In some embodiments, a self-amplifying RNA (saRNA) of the disclosure is encoded by the following expression vector, or a sequence with at least 70%, at least 80%, at least 90%, at least 95%, at least 96%, at least 97%, at least 98%, or at least 99% sequence identity thereto. In some embodiments, an saRNA of the disclosure is encoded by the following expression vector with a different gene of interest (GOI) sequence, following the organization presented in FIG. 30A as a Venezuelan equine encephalitis virus (VEEV) based replicon, or a sequence with at least 70%, at least 80%, at least 90%, at least 95%, at least 96%, at least 97%, at least 98%, or at least 99% sequence identity thereto. In some embodiments, an saRNA of the disclosure is encoded by the following expression vector with a different gene of interest (GOI) sequence, following the organization presented in FIG. 30A as a Venezuelan equine encephalitis virus (VEEV) based replicon, or a sequence with at least 70%, at least 80%, at least 90%, at least 95%, at least 96%, at least 97%, at least 98%, or at least 99% sequence identity thereto:
5′CSE: (SEQ ID NO: 79),
NSP1-4 & SGP cassette: (SEQ ID NO: 80),
GOI/antigen cassette (example: Firefly luciferase): (SEQ ID NO: 81),
Internal Ribosome Entry Site (IRES): (SEQ ID NO: 82),
EG Tech L protein coding sequence: (SEQ ID NO: 83), and
3′CSE & PolyA: (SEQ ID NO: 84).

In some embodiments, an saRNA of the disclosure is encoded by the following expression vector, or a sequence with at least 70%, at least 80%, at least 90%, at least 95%, at least 96%, at least 97%, at least 98%, or at least 99% sequence identity thereto. In some embodiments, an saRNA of the disclosure is encoded by the following expression vector with a different gene of interest (GOI) sequence, following the organization presented in FIG. 30A as a Semliki Forest Virus (SFV) based replicon, or a sequence with at least 70%, at least 80%, at least 90%, at least 95%, at least 96%, at least 97%, at least 98%, or at least 99% sequence identity thereto:
5′CSE: (SEQ ID NO: 85),
NSP & SGP cassette: (SEQ ID NO: 86),
GOI/antigen cassette (example: mKate2): (SEQ ID NO: 87),
Internal Ribosome Entry Site (IRES): (SEQ ID NO: 88),
EG Tech L protein coding sequence: (SEQ ID NO: 89),
Part A of SFV 26S RNA: (SEQ ID NO: 90), and
Part B of SFV 26S RNA, 3′CSE and polyA: (SEQ ID NO: 91).

In some embodiments, an saRNA of the disclosure is encoded by the following expression vector, or a sequence with at least 70%, at least 80%, at least 90%, at least 95%, at least 96%, at least 97%, at least 98%, or at least 99% sequence identity thereto. In some embodiments, an saRNA of the disclosure is encoded by the following expression vector with a different gene of interest (GOI) sequence, following the organization presented in FIG. 30A as a Venezuelan equine encephalitis virus (VEEV) based replicon, or a sequence with at least 70%, at least 80%, at least 90%, at least 95%, at least 96%, at least 97%, at least 98%, or at least 99% sequence identity thereto:
5′CSE: (SEQ ID NO: 92),
NSP1-4 & SGP cassette: (SEQ ID NO: 93),
GOI/antigen cassette (example: full-length influenza HA as an antigen): (SEQ ID NO: 94),
Internal Ribosome Entry Site (IRES): (SEQ ID NO: 95),
EG Tech L protein coding sequence: (SEQ ID NO: 96), and
3′CSE & PolyA: (SEQ ID NO: 97).

### RNA Modifications

The RNA provided herein, e.g., a messenger RNA or a self-amplifying RNA for the expression of a target protein and an NCT inhibitor protein may be modified as described herein. For example, an RNA may be modified to reduce immunogenicity in the cell or tissue. An RNA as provided herein may comprise one or more of an untranslated region (UTR), a 5′ cap, and a poly-adenosine tail.

In some embodiments, an RNA may be synthesized as an unmodified or modified RNA. An RNA may be modified to enhance stability and/or evade immune detection and degradation. A modified RNA may include, for example, one or more of a nucleotide modification, a nucleoside modification, a backbone modification, a sugar modification, and/or a base modification. In some embodiments, the modified nucleoside is pseudouridine or a pseudouridine analog. In some embodiments, the pseudouridine analog is N-1-methylpseudouridine.

In some embodiments, uracil nucleosides of the RNA are about 10%, about 20%, about 30%, about 40%, about 50%, about 60%, about 70%, about 80%, about 90%, 95%, 99%, or 100% depleted and replaced with a uracil nucleoside analog, e.g., pseudouridine, 5-methoxyuridine, or N-1-methyl-pseudouridine.

In some embodiments, an RNA may contain an RNA backbone modification. Typically, a backbone modification is a modification in which the phosphates of the backbone of the nucleotides contained in the RNA are chemically modified. Example backbone modifications may include, but are not limited to, modifications in which the phosphodiester linkage is replaced with a member from the group consisting of peptides, methylphosphonates, methylphosphoramidates, phosphoramidates, phosphorothioates (e.g., cytidine 5′-0-(l-thiophosphate)), boranophosphates, and/or positively charged guanidimum groups, or other means of replacing the phosphodiester linkage.

### Cap Structure

In some embodiments, an RNA may include a 5′ cap structure. A 5′ cap may comprise for example, a triphosphate linkage and a guanine nucleotide in which the 7-nitrogen is methylated. In some embodiments, the 5′ cap is 5′ 7-methyl guanosine (m7G). A variety of m7G cap analogs are known in the art, many of which are commercially available. These include the m7 GpppG described above, as well as the ARCA 3′—OCH3 and 2′—OCH3 cap analogs (Jemielity, J. et al., RNA, 9:1108-1122 (2003)). Additional cap analogs for use in embodiments of the invention include N7-benzylated dinucleoside tetraphosphate analogs (described in Grudzien, E. et at, RNA, 10:1479-1487 (2004)), phosphorothioate cap analogs (described in Grudzien-Nogalska, E., et al, RNA, 13:1745-1755 (2007)), and cap analogs (including biotinylated cap analogs) described in U.S. Pat. Nos. 8,093,367 and 8,304,529, incorporated herein by reference.

In some embodiments, the 5′ cap comprises or consists of an internal ribosome entry site (IRES). In some embodiments the IRES is within the 5′ UTR. In some embodiments, the 5′ cap comprises or consists of a 2A self-cleavage peptide, e.g, one or more of P2A, T2A, E2A and F2A.

### Tail Structure

The presence of a “tail” may serve to protect an RNA from exonuclease degradation. The poly-A tail is thought to stabilize natural messengers and synthetic sense RNA. Therefore, in certain embodiments a long poly-A tail can be added to an RNA molecule thus rendering the RNA more stable. Poly-A tails can be added using a variety of art-recognized techniques. For example, long poly-A tails can be added to synthetic or in vitro transcribed RNA using poly-A polymerase (Yokoe, et al. Nature Biotechnology. 1996; 14:1252-1256). A transcription template can also encode long poly-A tails. In addition, poly-A tails can be added by transcription directly from PCR products. Poly-A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

In some embodiments, an RNA may include a 3′ poly(A) tail structure. The length of the poly-A tail may be at least about 5, about 10, about 20, about 30, about 40, about 50, about 60, about 70, about 80, about 100, about 200, about 300, about 400, or about 500 nucleotides in length.

### 5′ and 3′ Untranslated Regions (UTRs)

In some embodiments, an RNA may include a 5′ untranslated region (UTR) and/or a 3′ UTR. In some embodiments, a 5 ‘UTR may include one or more elements that affect the stability or translation of an RNA. In some embodiments, a 5’ UTR may be between about 50 to about 100, or from about 50 to about 500 nucleotides in length. In some embodiments, a 3′ UTR includes one or more of a poly-A signal, a binding site for proteins that may affect RNA stability or localization, or one or more binding sites for miRNAs. In some embodiments, a 3′ UTR may be between about 0 and about 50 nucleotides, or about 50 to about 100 nucleotides in length.

Example 3′ and 5′ UTR sequences may be derived from RNAs with relatively long half-lives (e.g., globin, actin, GAPDH, tubulin, histone, or citric acid cycle enzymes) to increase the stability of the sense RNA molecule. For example, a 5′ UTR sequence may include a partial sequence of a cytomegalovirus (CMV) immediate-early 1 (IE1) gene, or a fragment thereof to improve the nuclease resistance and/or improve the half-life of the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion. In some embodiments, a UTR may improve tissue specific expression. Example UTRs include hBG 5′ UTR (SEQ ID NO: 98) and hBG 3′UTR (SEQ ID NO: 102).

### DNA Templates for RNA Production In Vitro

RNAs of the present disclosure, e.g. encoding an NCT inhibitor protein or a target protein, may be produced by in vitro transcription (IVT) of one or more DNA templates having polynucleotide sequence(s) encoding the desired RNA. The DNA template may comprise one or more promoters that enable transcription. In some embodiments, for example, a template may comprise a T7 promoter configured for transcription of the target protein-encoding nucleotide sequence and/or the NCT inhibitor protein encoding nucleotide sequence. Alternatively, the RNA may be prepared synthetically.

A template may comprise a separating element for separate expression of the proteins. In various embodiments, the template is a bicistronic template or a polycistronic template. The separating element may be an internal ribosomal entry site (IRES) or 2A element. In some embodiments, a template may comprise a nucleic acid encoding a 2A self-cleaving peptide. Illustrative 2A self-cleaving peptides include P2A, E2A, F2A, and T2A.

In some embodiments, the first polynucleotide or the second polynucleotide, or both, are operatively linked to an internal ribosome entry site (IRES).

In some embodiments, the first polynucleotide or the second polynucleotide, or both, are operatively linked to a 2A element.

In some embodiments, the first polynucleotide or the second polynucleotide, or both, are operatively linked with a stem-loop structure that induces translation stop codon readthrough. Illustrative stem-loop structures are described in Napthine et al. RNA 18:241-52 (2012) and Houck-Loomis et al. Nature 480:561-64 (2011).

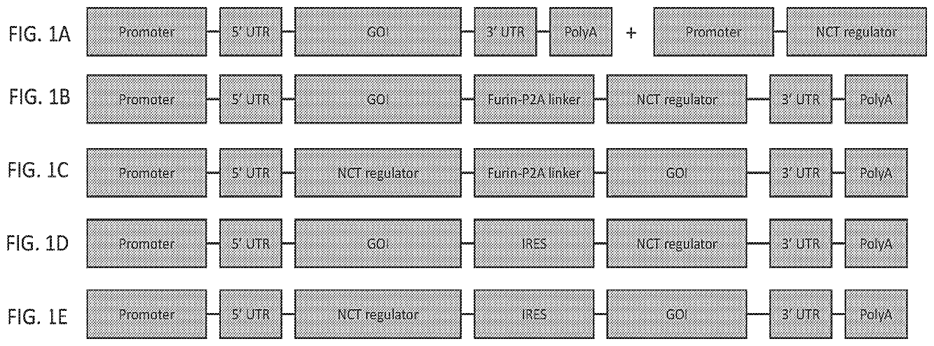

In some embodiments, the DNA templates encoding the RNAs of the disclosure may comprise or encode the following sequences in the sequence order presented in any one of FIG. 1A-1E, or a sequence with at least 70%, at least 80%, at least 90%, at least 95%, at least 96%, at least 97%, at least 98%, or at least 99% sequence identity thereto. In some embodiments, the DNA templates encoding the RNAs of the disclosure may comprise or encode the following sequences in the sequence order presented in any one of FIG. 1A-1E, or a sequence with at least 70%, at least 80%, at least 90%, at least 95%, at least 96%, at least 97%, at least 98%, or at least 99% sequence identity thereto, but encode a different target protein sequence:
T7 promoter SEQ ID NO: 70,
5′ UTR SEQ ID NO: 71,
3′ UTR SEQ ID NO: 72,
polyA tract SEQ ID NO: 73,
Nano-luciferase—Nucleotide target protein sequence SEQ ID NO: 74,
Nano-luciferase—Amino acid sequence SEQ ID NO: 75,
SARS-COV-2 spike-Nucleotide target protein sequence SEQ ID NO: 76,
SARS-COV-2 spike-Amino acid sequence SEQ ID NO: 15,
GBA signal peptide-Nucleotide target protein sequence SEQ ID NO: 77, and
GBA mature protein-Nucleotide target protein sequence SEQ ID NO: 78.

### V. DNA Compositions

Any DNA sequence encoding a target protein may be used with the methods and compositions described herein, i.e., an RNA encoding an NCT inhibitor protein.

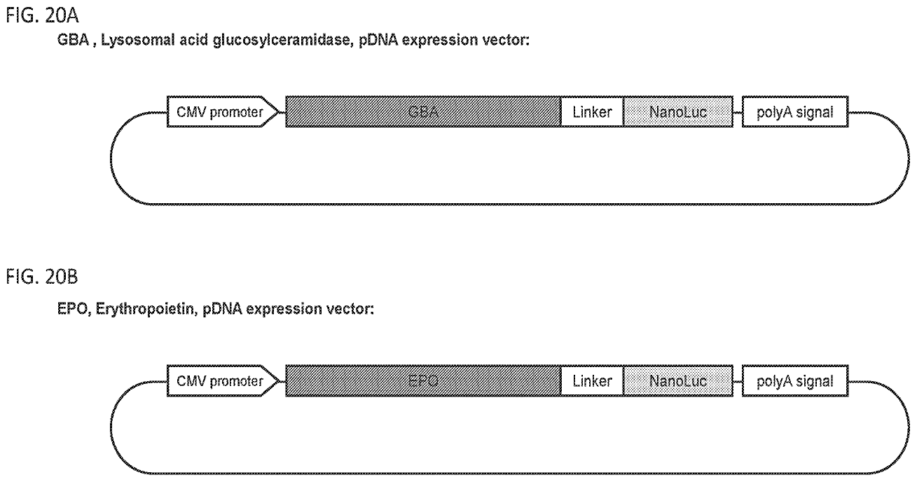

In some embodiments, a plasmid expression vector may comprise the following sequences in the sequence order presented in FIGS. 20A and 20B, or a sequence with at least 70%, at least 80%, at least 90%, at least 95%, at least 96%, at least 97%, at least 98%, or at least 99% sequence identity thereto. In some embodiments, a plasmid expression vector, may comprise the following sequences in the sequence order presented in FIGS. 20A and 20B, or a sequence with at least 70%, at least 80%, at least 90%, at least 95%, at least 96%, at least 97%, at least 98%, or at least 99% sequence identity thereto, but encode a different NCT inhibitor protein or target protein sequence:
GBA-NanoLuc/EPO Nano-Luc expression plasmid sequence, expression cassette:
CMV enhancer and promoter sequence: (SEQ ID NO: 108),
Multiple cloning site (MCS) sequence: (SEQ ID NO: 109),
GBA coding sequence: (SEQ ID NO: 110) or EPO coding sequence: (SEQ ID NO: 116),
Linker sequence: (SEQ ID NO: 111),
NanoLuciferase reporter sequence: (SEQ ID NO: 112), and
3′ UTR and SV40 poly(A) signal sequence: (SEQ ID NO: 113).

### VI. Delivery Vehicles

In some embodiments, one or more RNA and/or may be delivered to a cell or tissue via delivery vehicles. In some embodiments the delivery vehicle is a viral delivery vehicle. In some embodiments the delivery vehicle is a non-viral delivery vehicle.

### Non-Viral Delivery Vehicles

In some embodiments a delivery vehicle may be a nanoparticle. In some embodiments, the delivery vehicle is a lipid nanoparticle (LNP) including but not limited to a nanoparticle comprising lipids and/or polymers, a liposome, a liposomal nanoparticle, a cationic lipid, or an exosome. In other embodiments, the delivery vehicle may comprise calcium phosphate nucleotides, aptamers, cell-penetrating peptides or other vectorial tags.

In some embodiments, the nanoparticle is a polymeric nanoparticle. In some embodiments, the nanoparticle is a metal nanoparticle.

In some embodiments, an RNA may be delivered via a lipid nanoparticle (LNP). Example LNPs may comprise one or more different lipids and/or polymers. In some embodiments, an LNP comprises one or more of ionizable or cationic lipids, neutral lipids, cholesterols, and/or stabilizing lipids (e.g., PEGylated lipids).

In some embodiments, an LNP may comprise an ionizable lipid. An ionizable lipid may refer to any of a number of lipid species that have a net positive charge at a selected pH, such as a physiological pH. An ionizable lipid may also, for example, refer to a lipid in an ionized state, e.g., a cationic lipid, e.g., one or more of 2,2-dilinoleyl-4-dimethylaminoethyl-[1,3]-dioxolane (DLin-KC2-DMA), N-[1-(2,3-dioleyloxy) propyl]-N,N,N-trimethylammonium chloride (DOTMA), 5-carboxyspermylglycinedioctadecylamide (DOGS), 2,3-dioleyloxy-N-[2 (spermine-carboxamido) ethyl]-N,N-dimethyl-1-propanaminium (DOSPA), 1,2-Diolcoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane or (DLenDMA), N-dioleyl-N,N-dimethylammonium chloride (DODAC), or variations thereof.

In some embodiments, an LNP may comprise a neutral or zwitterionic lipid. In some embodiments the neutral lipid is selected from one more of: distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), diolcoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or variations thereof.

In some embodiments, the LNP comprises a PEGylated lipid, a cholesterol, and one or more ionizable lipids. An example LNP formulation comprises about 1.5% DMG-PEG (2000), 38.5% cholesterol, 10% DSPC, and 50% DLin-MC3-DMA.

Lipid nanoparticles (LNPs) or liposomes carrying RNA may be produced, for example, by mixing the lipids or polymers in an organic solvent, e.g., ethanol, with one or more RNAs in an aqueous buffer, and then purifying the resulting nanoparticles by filtration. In some embodiments, the LNP or liposome may be produced using a microfluidic device to rapidly mix reagents and form monodisperse particles of controlled size. In some embodiments, the LNP or liposome may encapsulate an RNA and/or associate with one or more RNAs through electrostatic interactions.

### Viral Delivery Vehicles

In other embodiments, the delivery vehicle comprises a recombinant virus or virus-like particle, e.g., an adenovirus, adeno-associated virus (AAV), herpesvirus, or retrovirus, e.g., lentivirus. In some embodiments, the delivery vehicle comprises a modified viral vector, e.g., an adenovirus dodecahedron or recombinant adenovirus conglomerate.

### VII. Methods of Treatment

Methods of treatment as described herein refer to the treatment of a disease or disorder in a subject in need thereof by administration of one or more RNAs for the expression of a target protein with an NCT inhibitor protein, e.g. an L protein. In some embodiments, an RNA encoding an NCT inhibitor protein is a messenger RNA. In some embodiments, an RNA encoding an NCT inhibitor protein is a self-amplifying RNA.

In some embodiments, the RNA or DNA provided herein for the expression of a target protein with an RNA encoding an NCT inhibitor protein, e.g. an L protein, encode an adalimumab protein, CDR sequence, or variable light or heavy chain sequence. In some embodiments, RNA or DNA encoding an adalimumab protein or sequence and an RNA encoding an NCT inhibitor protein may treat one or more of rheumatoid arthritis, psoriatic arthritis, ankylosing spondylitis, Crohn's disease, ulcerative colitis, psoriasis, hidradenitis suppurativa, uveitis, and juvenile idiopathic arthritis.

In some embodiments, the RNAs of the disclosure are associated with an immune reaction, and may be used as a vaccine. In some embodiments, the vaccine is used to treat or prevent a disease or infection associated with the pathogen from which the antigen protein is derived, or from a related pathogen. In some embodiments, the antigen protein is a viral antigen protein. As provided herein, an RNA or DNA of the disclosure, encoding a target protein, may encode any of the viral antigen proteins of Table 3 to be administered with an RNA encoding an NCT inhibitor protein as a vaccine. In some embodiments, the RNA or DNA encoding a target protein of the disclosure provides a vaccine against a Coronavirus, e.g., severe acute respiratory syndrome (SARS), Middle East respiratory syndrome (MERS), or West Nile Virus. In some embodiments, the RNA or DNA of the disclosure encoding a target protein provides a vaccine against influenza virus, Hepatitis B virus, Human Papilloma virus (HPV), West Nile virus, or Human Immunodeficiency Virus (HIV) virus.

In some embodiments, an RNA encoding an L protein, e.g., a messenger RNA or a self-amplifying RNA is administered to a cell in a concentration of about 0.01 ng, about 0.05 ng, about 0.1 ng, about 0.15 ng, about 0.2 ng, about 0.3 ng, about 0.4 ng, about 0.5 ng, about 0.6 ng, about 0.7 ng, about 0.8 ng, about 0.9 ng, about 1.0 ng, about 1.1 ng, about 1.2 ng, about 1.3 ng, about 1.4 ng, about 1.5 ng, about 1.6 ng, about 1.7 ng, about 1.8 ng, or about 2.0 ng per well of a 96 well plate, or an equivalent thereof.

In some embodiments, expression of the NCT inhibitor protein and the target protein increases the expression level of the target protein in a cell or a subject. In some embodiments, expression of the NCT inhibitor protein and the target protein increases the expression level of the target protein in a cell or a subject by about 2-fold, about 3-fold, about 4-fold, about 5-fold, about 6-fold, about 7-fold, about 8-fold, about 9-fold, about 10-fold, about 20-fold, about 30-fold, about 40-fold, 50-fold, about 60-fold, about 70-fold, about 80-fold, about 90-fold, about 100-fold, about 200-fold, about 500 fold, or about 1000-fold.

In some embodiments, an RNA encoding an NCT inhibitor protein reduces the expression level of a target protein in a cell or a subject. In some embodiments, expression of the NCT inhibitor protein and the target protein reduces the expression level of the target protein in a cell or a subject by about 10%, about 20%, about 30%, about 40%, about 50%, about 60%, about 70%, about 80%, or about 90%.

In some embodiments, an RNA encoding an NCT inhibitor protein increases the expression level of a target protein in a cell or a subject by about 10%, about 20%, about 30%, about 40%, about 50%, about 60%, about 70%, about 80%, about 90%, about 100% about 200%, about 300%, about 400%, about 500%, or about 1000%,

In some embodiments of the disclosure, an RNA encoding an NCT inhibitor protein increases the activity of the target protein in a cell of the subject or the subject. In some embodiments, expression of the NCT inhibitor protein and the target protein increases the activity of the target protein in a cell of the subject or the subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

In some embodiments of the disclosure, an RNA encoding an NCT inhibitor protein increases the duration of time in which the target protein is found in a cell of the subject or the subject. In some embodiments, an RNA encoding an NCT inhibitor protein increases the duration of time in which the target protein is found in a cell of the subject or the subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20-fold.

In some embodiments of the disclosure, an RNA encoding an NCT inhibitor protein reduces the coefficient of variation (CV %) of the target protein in the tissue of the subject or the subject. In some embodiments, an RNA encoding an NCT inhibitor protein reduces the coefficient of variation (CV %) of the target protein in the tissue of the subject or the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

In some embodiments of the disclosure, an RNA encoding an NCT inhibitor protein reduces the degradation of the target protein. In some embodiments, an RNA encoding an NCT inhibitor protein reduces the degradation of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

### Routes of Administration

In some embodiments, one or more an RNAs encoding an NCT inhibitor protein, e.g. an L protein, may be delivered orally, subcutaneously, intravenously, intranasally, intradermally, transdermally, intraperitoneally, intramuscularly, intrapulmonary, vaginally, rectally, or intraocularly. In example embodiments RNAs may be administered intramuscularly or subcutaneously.

### VIII. Kits

Also provided herein are kits comprising an RNA encoding an NCT inhibitor protein of the disclosure for the improvement of target protein expression. The kit may comprise for example, a suitable container, e.g., a vial or tube, and instructions for use thereof.

In some embodiments, a kit of the disclosure comprises an RNA encoding an NCT inhibitor, e.g., an L protein, comprising a sequence of Table 4, a sequence of SEQ ID NO: 161, a mutant sequence of any one of 163, 164, 166, or 168, or a sequence with about 70%, about 80%, about 90%, about 91%, about 92%, about 93%, about 94%, about 95%, about 96%, about 97%, about 98%, or about 99% sequence identity thereto.

In some embodiments, a kit of the disclosure comprises an RNA encoding an NCT inhibitor packaged in, or together with, a delivery vehicle, e.g, an LNP, or delivery vehicle. In some embodiments of the disclosure, a kit comprising an RNA encoding an L protein, e.g., a messenger RNA or a self-amplifying RNA, is present in a vial or tube in an amount of about 0.01 ng, about 0.05 ng, about 0.1 ng, about 0.15 ng, about 0.2 ng, about 0.3 ng, about 0.4 ng, about 0.5 ng, about 0.6 ng, about 0.7 ng, about 0.8 ng, about 0.9 ng, about 1.0 ng, about 1.1 ng, about 1.2 ng, about 1.3 ng, about 1.4 ng, about 1.5 ng, about 1.6 ng, about 1.7 ng, about 1.8 ng, or about 2.0 ng, for use per well of a 96 well plate, or equivalents thereof.

### ENUMERATED EMBODIMENTS

Embodiment I-1. A method of improving expression of a target protein in a cell or subject, comprising administering one or more ribonucleic acids (RNA) encoding a target protein and an NCT inhibitor protein.

Embodiment I-2. The method of embodiment I-1, wherein the NCT inhibitor protein has at least at least 70%, at least 75%, at least 80%, at least 85%, at least 90%, at least 95%, at least 96%, at least 97%, at least 98%, or at least 99% sequence identity to an NCT inhibitor protein amino acid sequence of Table 4.

Embodiment I-3. The method of embodiment I-1, wherein the NCT inhibitor protein has at least 70%, at least 75%, at least 80%, at least 85%, at least 90%, at least 95%, at least 96%, at least 97%, at least 98%, or at least 99% sequence identity to the L protein sequence of SEQ ID NO: 2.

Embodiment I-4. The method of any one of embodiments I-1 to I-3, wherein the target protein is selected from any of the sequences of Tables 1, 2, and 3, or an amino acid sequence with at least 70%, at least 75%, at least 80%, at least 85%, at least 90%, at least 95%, at least 96%, at least 97%, at least 98%, or at least 99% sequence identity thereto.

Embodiment I-5. The method of any one of embodiments I-1 to I-4, wherein the target protein is an antibody.

Embodiment I-6. The method of embodiment I-5, wherein the target protein is adalimumab, wherein optionally the target protein comprises the adalimumab heavy chain amino acid sequence of SEQ ID NO: 13 or an amino acid sequence with at least 70%, at least 75%, at least 80%, at least 85%, at least 90%, at least 95%, at least 96%, at least 97%, at least 98%, or at least 99% sequence identity thereto; and/or the adalimumab light chain amino acid sequence of SEQ ID NO: 14 or an amino acid sequence with at least 70%, at least 75%, at least 80%, at least 85%, at least 90%, at least 95%, at least 96%, at least 97%, at least 98%, or at least 99% sequence identity thereto.

Embodiment I-7. The method of any one of embodiments I-1 to I-4, wherein the target protein is an antigen of a pathogen, optionally a SARS-COV-2 spike(S) protein, wherein optionally the target protein comprises the amino acid sequence of SEQ ID NO: 15 or an amino acid sequence with at least 70%, at least 75%, at least 80%, at least 85%, at least 90%, at least 95%, at least 96%, at least 97%, at least 98%, or at least 99% sequence identity thereto.

Embodiment I-8. The method of any one of embodiments I-1 to I-7, wherein the method comprises delivery of the one or more RNAs with a lipid nanoparticle (LNP).

Embodiment I-9. The method of embodiment I-8, wherein the LNP comprises a PEGylated lipid, a cholesterol, and a neutral lipid, and an ionizable lipid.

Embodiment I-10. The method of embodiment I-8, wherein the LNP comprises DMG-PEG (2000), cholesterol, DOPC and DLin-KC2-DMA in a ratio of about 1% to about 5% DMG-PEG (2000), about 30% to about 50% cholesterol, about 5% to about 15% DOPC, and about 40% to about 60% DLin-KC2-DMA.

Embodiment I-11. A method of treating a subject in need thereof for a disease or disorder, comprising administering to the subject one or more messenger ribonucleic acids (RNA) encoding a target protein and an NCT inhibitor protein.

Embodiment I-12. The method of embodiment I-11, wherein the NCT inhibitor protein has at least at least 70%, at least 75%, at least 80%, at least 85%, at least 90%, at least 95%, at least 96%, at least 97%, at least 98%, or at least 99% sequence identity to an NCT inhibitor protein amino acid sequence of Table 4.

Embodiment I-13. The method of embodiment I-11, wherein the NCT inhibitor protein has at least 70%, at least 75%, at least 80%, at least 85%, at least 90%, at least 95%, at least 96%, at least 97%, at least 98%, or at least 99% sequence identity to the L protein sequence of SEQ ID NO: 2.

Embodiment I-14. The method of any one of embodiments I-11 to I-13, wherein the target protein is selected from any of the sequences of Tables 1, 2, and 3, or an amino acid sequence with at least 70%, at least 75%, at least 80%, at least 85%, at least 90%, at least 95%, at least 96%, at least 97%, at least 98%, or at least 99% sequence identity thereto.

Embodiment I-15. A method of vaccinating a subject in need thereof, comprising administering to the subject one or more messenger ribonucleic acids (RNA) encoding a target protein and an NCT inhibitor protein, wherein the target protein is an antigen protein.

Embodiment I-16. The method of embodiment I-15, wherein the NCT inhibitor protein has at least at least 70%, at least 75%, at least 80%, at least 85%, at least 90%, at least 95%, at least 96%, at least 97%, at least 98%, or at least 99% sequence identity to an NCT inhibitor protein amino acid sequence of Table 4.

Embodiment I-17. The method of embodiment I-15 or I-16, wherein the antigen protein is selected from any of the sequences of Table 3, or an amino acid sequence with at least 70%, at least 75%, at least 80%, at least 85%, at least 90%, at least 95%, at least 96%, at least 97%, at least 98%, or at least 99% sequence identity thereto.

Embodiment I-18. The method of any of the preceding embodiments, wherein co-expression of the NCT inhibitor protein and the target protein increases the expression level of the target protein in a cell or a subject by about 2-fold, about 3-fold, about 4-fold, about 5-fold, about 6-fold, about 7-fold, about 8-fold, about 9-fold, about 10-fold, about 20-fold, about 30-fold, about 40-fold, 50-fold, about 60-fold, about 70-fold, about 80-fold, about 90-fold, about 100-fold, about 200-fold, about 500 fold, or about 1000-fold.

Embodiment I-19. The method of any of the preceding embodiments, wherein co-expression of the NCT inhibitor protein and the target protein reduces the expression level of the target protein in a cell or a subject by about 10%, about 20%, about 30%, about 40%, about 50%, about 60%, about 70%, about 80%, or about 90%.

Embodiment I-20. The method of any of the preceding embodiments, wherein co-expression of the NCT inhibitor protein and the target protein increases the activity of the target protein in a cell of the subject or the subject by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment I-21. The method of any of the preceding embodiments, wherein co-expression of the NCT inhibitor protein and the target protein increases the duration of time in which the target protein is found in a cell of the subject or the subject by about 2-fold, about 3-fold, about 4-fold, about 5-fold, about 6-fold, about 7-fold, about 8-fold, about 9-fold, about 10, about 11-fold, about 12-fold, about 13-fold, about 14-fold, about 15-fold, about 16-fold, about 17-fold, about 18-fold, about 19-fold, or about 20-fold.

Embodiment I-22. The method of any of the preceding embodiments, wherein co-expression of the NCT inhibitor protein and the target protein decreases the coefficient of variation (CV %) of the target protein in the tissue of the subject or the subject by about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.7-fold, about 2.8-fold, about 2.9-fold, or about 3-fold.

Embodiment I-23. The method of any of the preceding embodiments, wherein co-expression of the NCT inhibitor protein and the target protein reduces the degradation of the target protein by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment I-24. The method of any of the preceding embodiments, wherein co-expression of the NCT inhibitor protein and the target protein reduces the EC50 of target by about 10-fold, about 20-fold, about 30-fold, about 40-fold, about 50-fold, about 60-fold, about 70-fold, about 80-fold, about 90-fold, about 100-fold, about 150-fold, about 200-fold, or about 300-fold.

Embodiment I-25. A composition, comprising one or more ribonucleic acids (RNA) encoding a target protein and an NCT inhibitor protein.

Embodiment I-26. The composition of embodiment I-24, wherein the NCT inhibitor protein has at least at least 70%, at least 75%, at least 80%, at least 85%, at least 90%, at least 95%, at least 96%, at least 97%, at least 98%, or at least 99% sequence identity to an NCT inhibitor protein amino acid sequence of Table 4.

Embodiment I-27. The composition of embodiment I-24, wherein the NCT inhibitor protein has at least 70%, at least 75%, at least 80%, at least 85%, at least 90%, at least 95%, at least 96%, at least 97%, at least 98%, or at least 99% sequence identity to the L protein sequence of SEQ ID NO: 2.

Embodiment 1-28. The composition of any one of embodiments I-24 to I-27, wherein the target protein is selected from any of the sequences of Tables 1, 2, and 3, or an amino acid sequence with at least 70%, at least 75%, at least 80%, at least 85%, at least 90%, at least 95%, at least 96%, at least 97%, at least 98%, or at least 99% sequence identity thereto.

Embodiment I-29. The composition of any one of embodiments I-24 to I-27, wherein the target protein is an antibody.

Embodiment I-30. The composition of embodiment I-28, wherein the target protein is adalimumab, wherein optionally the target protein comprises the adalimumab heavy chain amino acid sequence of SEQ ID NO: 13 or an amino acid sequence with at least 70%, at least 75%, at least 80%, at least 85%, at least 90%, at least 95%, at least 96%, at least 97%, at least 98%, or at least 99% sequence identity thereto; and/or the adalimumab light chain amino acid sequence of SEQ ID NO: 14 or an amino acid sequence with at least 70%, at least 75%, at least 80%, at least 85%, at least 90%, at least 95%, at least 96%, at least 97%, at least 98%, or at least 99% sequence identity thereto.

Embodiment I-31. The composition of any one of embodiments I-24 to I-27, wherein the target protein is an antigen of a pathogen, optionally a SARS-COV-2 spike(S) protein, wherein optionally the target protein comprises the amino acid sequence of SEQ ID NO: 15 or an amino acid sequence with at least 70%, at least 75%, at least 80%, at least 85%, at least 90%, at least 95%, at least 96%, at least 97%, at least 98%, or at least 99% sequence identity thereto.

Embodiment I-32. The composition of any one of embodiments I-24 to I-30, wherein the target protein is selected from any of the sequences of Tables 1, 2, and 3, or an amino acid sequence with at least 70%, at least 75%, at least 80%, at least 85%, at least 90%, at least 95%, at least 96%, at least 97%, at least 98%, or at least 99% sequence identity thereto.

Embodiment I-33. The composition of any one of embodiments I-24 to I-31, wherein the method comprises delivery of the one or more RNAs with a lipid nanoparticle (LNP).

Embodiment I-34. The composition of embodiment I-32, wherein the LNP comprises a PEGylated lipid, a cholesterol, and a neutral lipid, and an ionizable lipid.

Embodiment I-35. The composition of embodiment I-32, wherein the LNP comprises DMG-PEG (2000), cholesterol, DOPC and DLin-KC2-DMA in a ratio of about 1% to about 5% DMG-PEG (2000), about 30% to about 50% cholesterol, about 5% to about 15% DOPC, and about 40% to about 60% DLin-KC2-DMA.

Embodiment I-36. A system comprising:
(i) one or more polynucleotides encoding an NCT inhibitor; and
(ii) a delivery vehicle.

Embodiment I-37. The system of embodiment I-36, wherein the polynucleotide encoding the NCT inhibitor is RNA.

Embodiment I-38. The system of embodiment I-36, wherein the delivery vehicle is a non-viral delivery vehicle.

Embodiment I-39. The system of embodiment I-38, wherein the non-viral delivery vehicle is an LNP.

Embodiment I-40. The system of embodiment I-36, wherein the delivery vehicle is a viral delivery vehicle.

Embodiment I-41. The system of embodiment I-36, comprising an RNA encoding a target protein.

Embodiment I-42. The system of embodiment I-41, wherein the RNA comprises at least one unmodified uridine nucleotide.

Embodiment I-43. The system of embodiment I-41, wherein the RNA is a self-amplifying RNA (saRNA).

Embodiment I-44. A method of reducing cell signaling in response to a stressor comprising administering a polynucleotide encoding an NCT inhibitor to a cell.

Embodiment I-45. The method of embodiment I-44, wherein the stressor is selected from the group consisting of: innate immune signaling; pro-inflammatory signaling; delivery vector induced inflammation; natural nucleoside mRNA transfection; self-amplifying RNA transfection; and expression of a target protein.

Embodiment I-46. The method of embodiment I-44, wherein the polynucleotide encoding the NCT inhibitor is RNA.

Embodiment I-47. The system of embodiment I-36 wherein the polynucleotide encoding the NCT inhibitor comprises a sequence of SEQ ID NOS: 65, 66, 67, or 68.

Embodiment I-48. The method of embodiment I-11, wherein the target protein is an antigen for use in a vaccine.

Embodiment I-49. The system of any one of embodiments I-41 to I-43, wherein the target protein is an antigen for use in a vaccine.

Embodiment I-50. The method of embodiment I-44, comprising co-expressing a target protein, wherein the target protein is an antigen for use in a vaccine.

### EXAMPLES

### Example 1: Methods of RNA Production

The compositions and methods of the present disclosure provide RNAs encoding a target protein (e.g., therapeutic proteins, viral proteins, antibodies, and reporter proteins) and a nuclear cytoplasmic transport (NCT) inhibitor proteins, e.g. an NCT inhibitor L protein. Inclusion of an RNA encoding an NCT inhibitor protein may provide one or more of: a) prolonged expression time of the target protein, b) reduced cellular inflammation in vitro, c) reduced inflammation in vivo, d) higher quality and/or quantity of the expressed target protein in vivo and in vitro, and/or c) improved cytokine secretion.

### Construction of RNA Molecules

All assemblies were made into a plasmid backbone capable of propagation in E. coli comprising a promoter controlling a high copy number origin of replication (ColE1) followed by a terminator (rrnB T1 and T2 terminator). This is followed by a promoter controlling an antibiotic resistance gene which is isolated from the rest of the plasmid template by a second terminator (transcription terminator from phage lambda). The genes comprising elements of the backbone were synthesized by phosphoramidite chemistry. FIGS. 1A-1E show the gene of interest (GOI) can be delivered on a separate RNA (FIG. 1A) from the NCT inhibitor, e.g. an NCT inhibitor L protein, and co-transfected, or on the same RNA (FIGS. 1B-1E). The NCT inhibitor, e.g. an L protein, on the same RNA may be either before or after the gene of interest in the 5′ to 3′ direction. A protease cleavage site, e.g. a furin site, may be present (FIG. 1B and FIG. 1C). An internal ribosomal entry site (IRES) may also be present (FIG. 1D and FIG. 1E).

Structure genes used for the construction of the plasmids were synthesized by phosphoramidite chemistry, chemistry, amplified and cloned into the vector described above using an isothermal assembly reaction such as NEB HI-FI or Gibson Assembly.

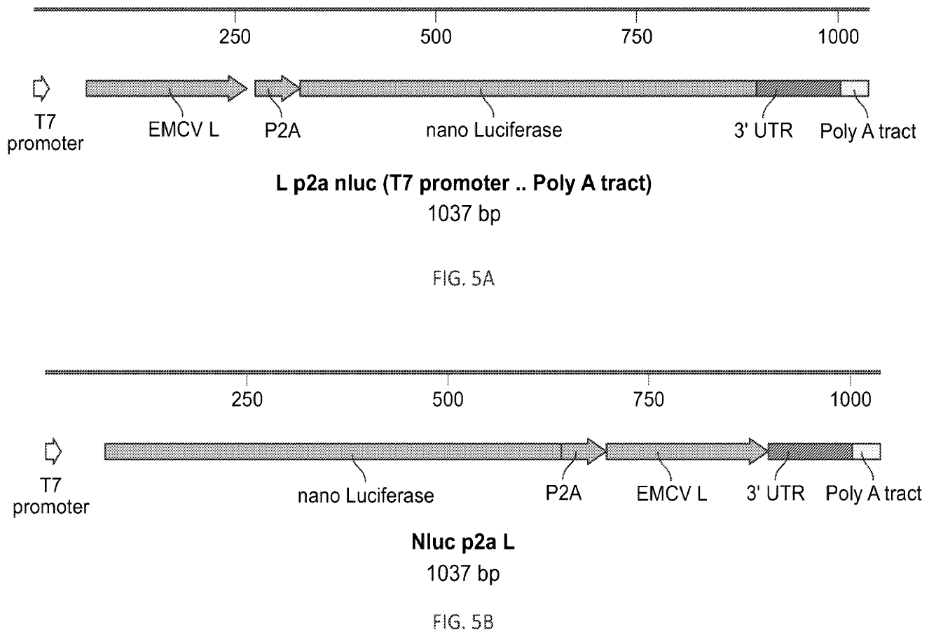

FIGS. 5A and 5B show DNA template construct configurations used for in vitro transcription (IVT) to generate single mRNA constructs encoding the L protein (L) and nano luciferase (nLuc). FIG. 5A shows a DNA template construct used for IVT to generate a single mRNA construct encoding L and nLuc separated by a p2A separating element (L-NLuc). FIG. 5B shows a DNA template construct used for IVT to generate a single mRNA construct encoding nLuc and L separated by a p2A separating element (NLuc-L).

### DNA Template Preparation

Fully cloned DNA templates contained the T7 promoter, 5′ UTR, ORF, 3′UTR, Poly A region, BSAI site. DNA templates were linearized by digest with BSAI (NEB r3733), linearized plasmid was separated from circularized plasmid by gel electrophoresis (1% agarose). The linear fragment was extracted by phenol/chloroform extraction with an equal volume of 1:1 phenol/chloroform. The DNA was precipitated by adding 1/10^{th} volume of 3M sodium acetate at pH 5.2, and two volumes of ethanol. DNA was incubated at −20 degrees centigrade for 30 minutes followed by pelleting the DNA for 15 minutes in a microcentrifuge. The DNA pellet was washed with 500 μl of 70% ethanol and centrifuged for 15 minutes at top speed. The ethanol was removed carefully and the pellet air dried before being resuspended in nuclease free water. In some cases, DNA templates for RNA synthesis were generated using PCR, instead of linearizing the fully cloned DNA using the BSAI restriction enzyme as described above. For these PCR reactions, the Forward primer was designed such that it would hybridize with the T7 promoter and the 3′UTR region of the fully cloned plasmid DNA template, ensuring that the 5′ end of the PCR amplicon would be compatible with the CleanCap reagent RNA capping reagent used downstream. The Reverse primers were designed to hybridize with the 3′UTR region of the fully cloned plasmid DNA template, ensuring the intact 3′UTR of the DNA template would be retained within the amplicon. The PCR-generated DNA templates were gel-purified using agarose gel electrophoresis, the Zymoclean Gel DNA Recovery Kit (Zymo Research) and the Monarch PCR & DNA Cleanup Kit (NEB) according to the manufacturer's instructions. DNA templates, generated via either method, were then used as templates for RNA synthesis, as described below.

### RNA Synthesis

To generate RNA from in vitro transcription, the HiScribe™ T7 ARCA RNA Kit, was used which generates 5′ 7-methyl guanosine (m7G) caps co-transcriptionally. In some examples, RNA in vitro transcription was conducted using the HiScribe T7 mRNA Kit with CleanCap Reagent AG Kit (NEB) with 100% N1-pseudouridine (Trilink) according to the manufacturer's instructions, using respective gel-purified PCR products as templates for RNA synthesis reactions. All components were thawed on ice prior to reaction assembly. The reaction was assembled in ice to the following specifications.

TABLE 5A
RNA synthesis reaction using the HiScribe T7 ARCA RNA Kit

| Reagent | Amount | Final concentration |
|---|---|---|
| Nuclease-free water | To 20 μL | |
| ARCA/NTP Mix (2x) | 10 μl | 1 mM GTP, 4 mM ARCA, |
| | | 1.25 mM CTP, 1.25 mM |
| | | UTP, >1.25 mM ATP |
| Pseudo-UTP, 10 mM | 2.5 μl | 1.25 mM |
| Template DNA | x μl | 1 ug |
| T7 RNA Polymerase Mix | 2 μl | |
| Total reaction volume | 20 μl | |

TABLE 5B
RNA synthesis reaction using the HiScribe
T7 mRNA Kit with CleanCap Reagent AG Kit

| Reagent | Amount | Final concentration |
|---|---|---|
| 10x reaction buffer | 2 μl | |
| ATP | 2 μl | 6 mM |
| GTP | 2 μl | 5 mM |
| CTP | 2 μl | 5 mM |
| N1-Methylpseudouridine | 2 μl | 4 mM |
| CleanCap AG | 2 μl | 4 mM |
| Nuclease free water | 1 μl | |
| DNA template (+water) | 6 μl | 0.1-0.5 μg |
| T7 RNA Polymerase Mix | 2 μl | |
| Total reaction volume | 20 μl | |

The reaction was mixed, pulse spun and incubated at 37° C. for 1-2 hours. To remove input DNA template, 2 uL of DNase I was added, mixed well, and incubated at 37 C for 15 min. The synthesized RNA was purified using the Monarch RNA Cleanup Kit (NEB). Then, the Poly(A) tailing reaction was assembled on ice to the following specifications:

TABLE 6
IVT RNA poly(A) tailing reaction

| Reagent | Amount |
|---|---|
| Nuclease-free water | 20 uL |
| IVT reaction | 20 μl |
| Poly(A) Polymerase reaction Buffer (10x) | 5 μl |
| Poly(A) Polymerase | 5 μl |
| Total reaction volume | 50 μl |

The reaction was mixed, pulse spun and incubated at 37° C. for 30 minutes. The Poly(A)-tailed RNA was purified using the Monarch RNA Cleanup Kit (NEB).

### Purification of RNA

As mentioned above, RNA was purified after the in vitro transcription step as well as after the Poly(A)-tailing step using the Monarch RNA Cleanup Kit (NEB) according to the manufacturer's instructions. In some cases, RNA was purified using Phenol Chloroform extraction and ethanol precipitation. The reaction was adjusted to 180 μl by adding nuclease water before adding 20 μl of 3 M sodium acetate, pH 5.2. The RNA was then extracted with an equal volume of 1:1 phenol/chloroform mixture, followed by two extractions with chloroform. The aqueous phase was collected and transferred to a new tube. The RNA was precipitated by adding 2 volumes of ethanol. The reaction was incubated at −20° C. for at least 30 minutes and the pellet collected by centrifugation. The supernatant was removed and the pellet was rinsed with 500 ul of cold 70% ethanol before resuspending in 50 μl of 0.1 mM EDTA and stored at −20.

### Cell Lines—Culturing and Transfection

HEK293 cells are used to illustrate the application of the present systems, methods, and compositions in human eukaryotic cells. HEK293 adherent cells (CLS) were cultured in Dulbecco's Modified Eagle Medium high glucose (Gibco) supplemented with 10% Fetal Bovine Serum (Gibco) and 50,000 Anti-Anti (Gibco). HEK293 cells were grown to 80% confluency at 37° C. and 5% CO₂ before transiently transfecting using lipofectamine 3000 (ThermoFisher) according to manufacturer's instruction. Protein-expressing cells were analyzed after 48 h as described in the examples.

Vero cells are used to illustrate the application of the present systems, methods, and compositions in eukaryotic animal cells. Vero E6 adherent cells (ATCC) were cultured in Dulbecco's Modified Eagle Medium high glucose (Gibco) supplemented with 10% Fetal Bovine Serum (Gibco) and 50,000 U Anti-Anti (Gibco). Vero cells were grown to 80% confluency at 37° C. and 5% CO₂ before transiently transfecting using lipofectamine 3000 (ThermoFisher) according to manufacturer's instruction. Protein-expressing cells were analyzed after 48 h as described in the examples.

C2C12 cells were used to illustrate improvement of cytokine sensitivity of the present systems, methods, and compositions in animal cells. C2C12 adherent cells (ATCC) were cultured in Dulbecco's Modified Eagle Medium high glucose (Gibco) supplemented with 10% Fetal Bovine Serum (Gibco) and 50,000 Anti-Anti (Gibco). C2C12 cells were grown to 80% confluency at 37° C. and 5% CO₂ before transiently transfecting using lipofectamine 3000 (ThermoFisher) according to manufacturer's instruction. Protein-expressing cells were analyzed after 48 h as described in the examples.

BJ Fibroblast cells were used to illustrate improvement of cytokine sensitivity of the present systems, methods, and compositions in human cells. BJ Fibroblast adherent cells (ATCC) were cultured in Dulbecco's Modified Eagle Medium high glucose (Gibco) supplemented with 10% Fetal Bovine Serum (Gibco) and 50,000 Anti-Anti (Gibco). C2C12 cells were grown to 80% confluency at 37° C. and 5% CO₂ before transiently transfecting using lipofectamine 3000 (ThermoFisher) according to manufacturer's instruction. Protein-expressing cells were analyzed after 48 h as described in the examples.

RNA transfection in vitro was generally conducted using the Mirus TransIT mRNA transfection kit (Mirus), unless indicated otherwise, according to manufacturer's instructions. For co-transfection experiments (i.e., when conducting dose titration studies using multiple RNA molecules in trans), all the indicated RNA molecules were mixed in Opti-MEM first. In these experiments, an inert filler RNA was used to make sure the total RNA concentration for each transfection was the same. The used RNA amounts are indicated in respective examples. Mirus TransIT Boost Reagent was added to the pre-diluted RNA samples. Thereafter, Mirus TransIT Transfection Reagent was added, tubes were gently flicked and inverted to mix well. Tubes were incubated for a maximum of 5 min at room temperature, followed by the addition of prewarmed complete DMEM media (Gibco) to ‘quench’ the reaction. Transfection complexes were then added to the cells. In the experiments where the cells were stimulated with Poly I:C or IFN-beta, the stimulants were added to the cells at indicated final concentrations within 5 minutes from the treatment of cells with the transfection complexes.

### LNP-RNA Production

An example LNP formulation as provided herein is: 1.5% DMG-PEG (2000), 38.5% cholesterol, 10% DSPC, and 50% DLin-MC3-DMA. To produce the LNP-mRNA formulation used in the experiments, the lipids were dissolved in ethanol. The RNA was dissolved in an aqueous acidification buffer, e.g., 50 mM sodium citrate (pH 4). The lipids were combined with the RNA solution by rapid mixing, for example at the volume ratio of 1:3 (lipid: RNA) at the Nitrogen to Phophate ratio of 6:1 (Nitrogens of the ionizable lipid relative to Phosphates of the mRNA). After the rapid mixing, the mixture was further diluted in a 1:1 ratio of pH 6 buffer, followed by diafiltration against 15 mL PBS using an Amicon Ultra spin filter with a 10 kDa molecular weight cutoff.

### Nanoluciferase Assays

For the NanoLuc assay, cell culture supernatants were collected to microcentrifuge tubes and cleared from cell debris by centrifugation. The cells, left adherent onto the 24-well plates, were lysed using 250 μl of 0.2% Triton X-100 in PBS at room temperature. 50 μl of cleared cell culture supernatants or 50 μl of cell lysate was loaded to opaque white 96-well microplates. The collected supernatant and cell lysate samples were assayed for NanoLuc expression using the Nano-Glo Luciferase Assay System (Promega) as per manufacturer's instructions. Briefly, 50 μl of fresh 1×Nano-Glo assay reagent in the respective assay buffer (Promega) was added to each well, incubated 2-5 minutes, followed by luminescence measurement using a Biotex Synergy LX plate reader. Cell lysates were further analysed for their protein concentration using the A660 reagent (Thermo Scientific) according to the manufacturer's instructions. Briefly, 40 μl of samples or standards were mixed with 150 μl A660 assay reagent, the absorbance at 660 nm was measured using a Biotek Synergy LX plate reader, and protein concentration was quantified based on the standard curve. The luminescence of cell lysates was normalized per μg cell lysate protein (i.e., yielding luminescence values in units of RLU/μg protein) while the luminescence of cell culture supernatants was normalized using the volume of supernatant used in the assay (i.e., yielding luminescence values in units of RLU/ml supernatant).

### Animals

For animal studies, C57BL/6 (Charles River Laboratories) or a corresponding disease model are used. Age is indicated in the examples. Animals of the same sex are group housed in polycarbonate cages containing appropriate bedding. Mice are identified with by either visible tattoos on the tail or by implantation of an electronic identification chip. Mice are allowed acclimation for at least 5 days prior to treatment to accustom the animals to the laboratory environment. Housing was set-up as described in the Guide for the Care and Use of Laboratory Animals with social housing and a chewing object for animal environmental enrichment. The targeted environmental conditions are a temperature of 19 to 25° C., a humidity between 30% to 70% and a light cycle of 12 h light and 12 h dark. Food (Lab Diet Certified CR Rodent Diet 5CR4) was provided ad libitum in form of pellets. Water is provided freely available in form of municipal tap water, treated by reverse osmosis and ultraviolet irradiation.

### Example 2: In Vitro Expression of Reporter Gene (Luciferase) from RNA with L Protein

To test the effects of the L protein in mRNA constructs, interferon producing mouse C2C12 and human BJ fibroblast cells were transfected with single mRNA constructs encoding both luciferase and the L protein (NCT inhibitor) protein separated by a p2A element (FIGS. 5A and 5B). These mRNA constructs contained the ARCA cap and a 50%-50% mixture of natural uridines and pseudo-uridines. Due to the use of natural uridines in the mRNA, these mRNA constructs are inherently pro-inflammatory as they can activate innate immune signaling pathways in transfected cells, as known in the art. Both positions of the NCT inhibitor relative to the gene of interest (5′ to the gene and 3′ to the gene) were tested, demonstrating a flexible approach of the position of the NCT inhibitor. Interestingly the position of the L protein also tuned the influence of the L protein on gene of interest expression.

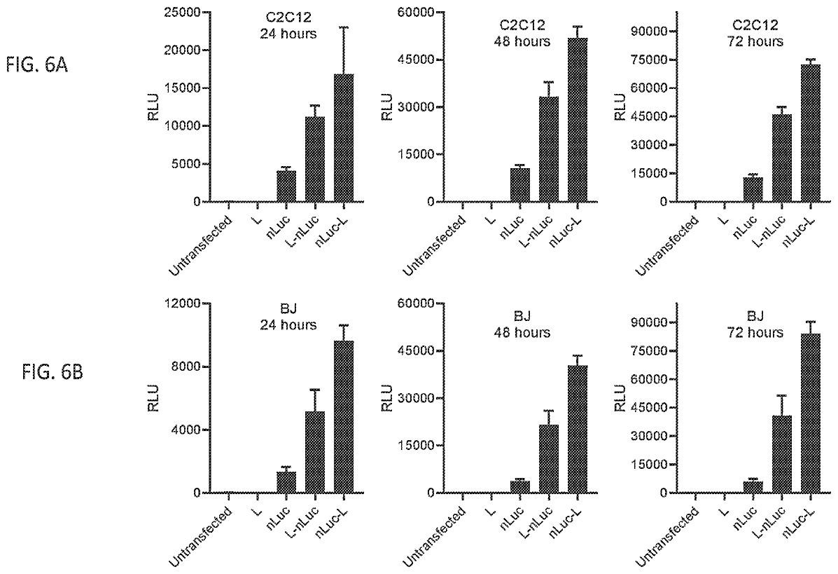

On Day 0, C2C12 and BJ Fibroblast cells were seeded in 96-well culture microplates in 200 μl DMEM (C2C12) or 200 μl MEM (BJ fibroblast) containing 10% FBS. On Day 1, the cells were transfected with the RNA constructs encoded by FIGS. 5A and 5B, using Lipofectamine™ MessengerMAX™ Transfection Reagent (Invitrogen) according to manufacturer's instructions. On the following days, the transfected cells and cell culture supernatants were assayed for the activity of NanoLuc luciferase 24 hours, 48 hours and 72 hours post transfection. FIGS. 6A and 6B show luciferase activity as a direct measurement of the expressed enzyme in mouse C2C12 and human BJ cells. Surprisingly, the co-expression of the L protein (NCT inhibitor) led to a more than 10 fold increase of expressed luciferase. This demonstrated the ability of the L protein to protect the expression of the gene of interest (GOI) against internal stress and inflammation signals induced by mRNA transfection.

To further investigate the role of the L protein in protecting protein expression against stress and inflammation signals, exogenous IFN-beta (a stress pathway stimulator) was added to the cells. Once a threshold concentration of external IFN-beta is reached, protein expression is known to be inhibited. To measure cell sensitivity to external IFN-beta with and without the L protein, C2C12 cells were transfected with mRNA encoding either the L protein alone (L), nLuc alone, or the two configurations of a single mRNA encoding L and nLuc as shown in FIGS. 5A (L-nLuc) and 5B (nLuc-L). 6 hours post transfection, a 10 fold dilution series of mouse recombinant IFN-beta was added to the C2C12 cells. luciferase activity was then measured 48 hours after IFN-beta stimulation. The approximate EC50 of IFN-beta required to reduce luciferase protein expression is measured as the concentration at which luciferase expression (as indicated by activity and normalized to no IFN-beta) was reduced by 50% (dotted line).

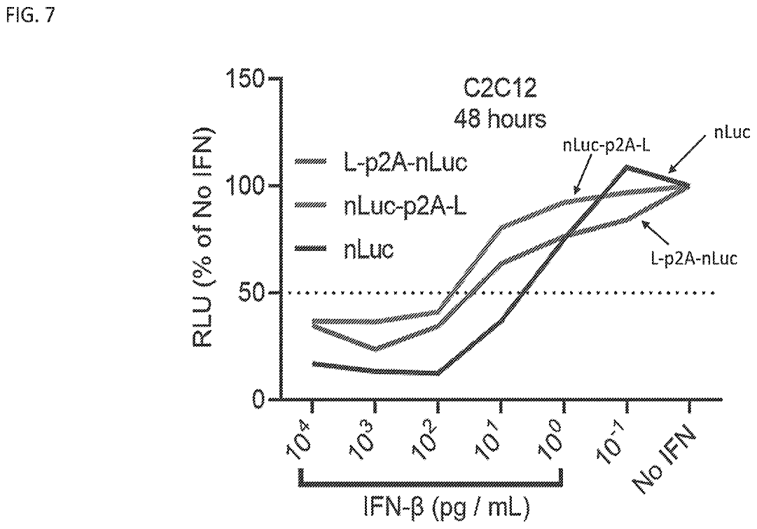

FIG. 7 shows decreased luciferase expression in the presence of IFN-beta. The approximate EC₅₀ of IFN-beta required to decrease luciferase protein expression for each construct is shown. As demonstrated by the increased approximate EC₅₀ values of IFN-beta, the presence of the L protein protected cells from external IFN-beta stimulation 10-fold and 12 fold (for L-nLuc and nLuc-L, respectively). Thus, the L protein protects protein expression from the external stress stimulation of IFN-beta.

The EC₅₀ value of IFN sensitivity is calculated in Prism (GraphPad) using the log (inhibitor) vs. response—variable slope four parameters nonlinear regression.

The secretion of IFN-beta was used as a marker for inflammation. For this, human BJ cells, which produce IFN-beta, were transfected with mRNA encoded by the constructs shown in FIGS. 5A and 5B. Supernatants were collected 24-72 hours post transfection and cleared by centrifugation at 500 g for 5 minutes. Clarified supernatants were then analysed for the presence of IFN-beta by ELISA (R&D Systems) according to the manufacturer's instructions.

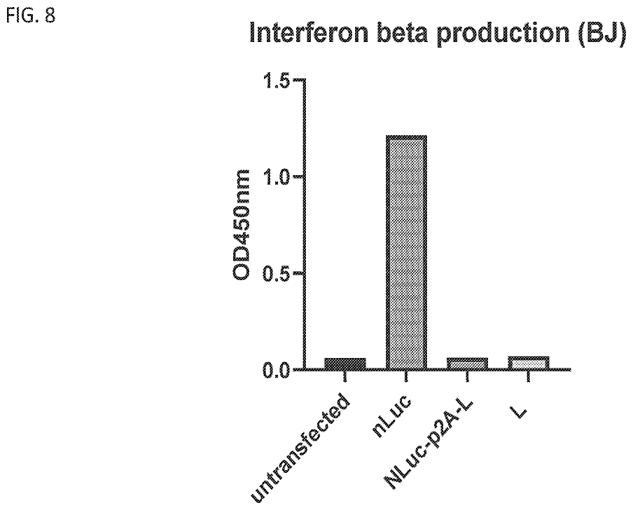

FIG. 8 shows the IFN-beta levels produced by the BJ cells after transfection with the mRNA constructs. Notably, the presence of the L protein prevented the BJ cells from producing INF-beta in response to transfection of mRNA. In the presence of L protein mRNA, RNA-intrinsic stress induced with 50% natural uridine containing pro-inflammatory mRNA, is not propagated by the cell, as exemplified by eliminating the cell's ability to upregulate IFN-beta expression and secretion as part of the stress response.

### Example 3: In Vitro Expression of Viral Protein (Spike Covid19) from RNA

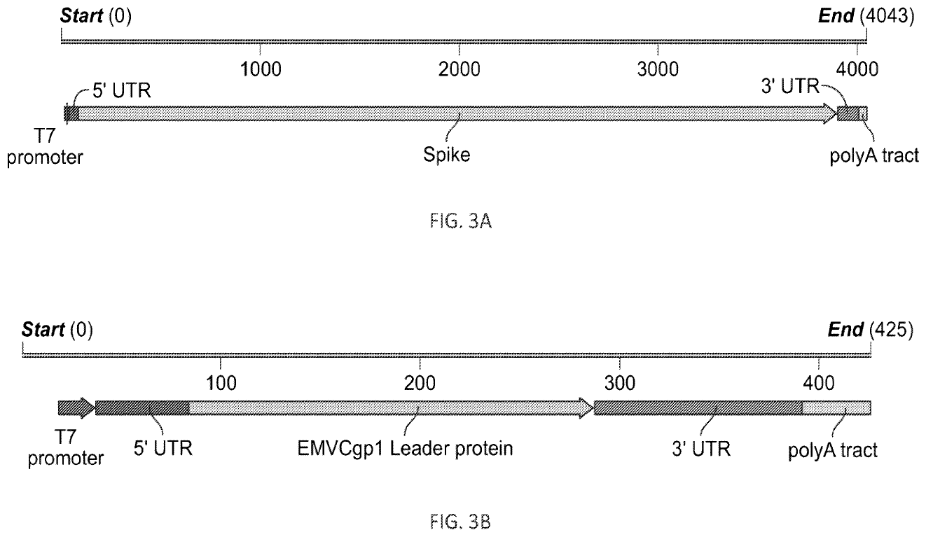

To test whether Sars-COV 2 Spike as a viral protein was expressed in HEK293T, Vero E6, C2C12 and/or BJ Fibroblast cells the following experiment is performed. HEK293T, Vero E6, C2C12 and/or BJ Fibroblast cells are transfected with RNA expressing Sars-CoV2 Spike alone as a control (FIG. 3A) and in parallel co-transfected with two RNA constructs, one expressing Sars-CoV2 Spike protein and the other expressing the NCT inhibitory protein (FIGS. 3A and 3B) from IVT reactions, as described next.

On Day 0, HEK293T, Vero E6, C2C12 and/or BJ Fibroblast cells are seeded in 24-well cell culture microplates in 500 μl DMEM (HEK293T, Vero E6, C2C12) or 500 μl MEM (BJ Fibroblast) containing 10% FBS. On Day 1, the cells are transfected with respective RNA-Spike alone or in combination with RNA-L from IVT reactions using Lipofectamine™ MessengerMAX™ Transfection Reagent (Invitrogen) according to manufacturer's instructions. On following days, the transfected cells and cell culture supernatants are assayed for the activity of NanoLuc luciferase.

Additionally, to measure the Interferon sensitivity with and without the NCT inhibitor protein, cells are treated with IFN-beta 6-24 h post transfection. For this mouse (for C2C12) or human (for HEK293T, Vero E6, BJ Fibroblast cells) recombinant IFN-beta (Fisher Scientific) was 5-fold serially diluted and added to cells 6 hours or 24 hours post transfection with RNA encoding Sars-CoV2 Spike protein alone or in combination with the NCT inhibitory protein. The ratio of Sars-CoV2 Spike expression is normalized to an untreated sample. The EC50 value of IFN sensitivity is calculated in Prism (GraphPad) using the log (inhibitor) vs. response-variable slope four parameters nonlinear regression.

For interferon producing cell lines (C2C12, BJ Fibroblast) the secretion of interferons as marker for inflammation is measured. For this cell supernatants were collected 24-72 hours post transfection and cleared by centrifuged at 500 g for 5 mins. Clarified supernatants are then analysed for the presence of IFN-beta by ELISA (R&D Systems) according to the manufacturer's instructions.

Additionally, ELISA assay is used to demonstrate the secretion of expressed Spike protein. High-binding 96-well plates are coated with cell culture supernatants isolated on Day 3 (after transfection) using 75 μl of cell culture supernatant per well and incubated at +4° C. overnight. The next day, the coated wells are washed using the PBST buffer and blocked using 200 ul of EZ Block reagent per well for 2 h at +37° C. The wells are washed 3× with PBST and incubated with a primary antibody (rabbit anti-RBD, diluted 1:500 in EZ Block, 75 ul per well) for 1 h at room temperature. The wells are then washed 3× with PBST and incubated with the goat anti-rabbit HRP secondary antibody diluted 1:1000 in EZ Block reagent, 75 ul per well, for 1 h at room temperature. The wells are then washed 5× using PBST and 75 ul of TMB substrate is added to each well and incubated 30 minutes at room temperature, followed by the addition of 75 μl of Stop Solution, and measuring the absorbance at 450 nm using a plate reader.

### Example 4: In Vitro Expression of GBA

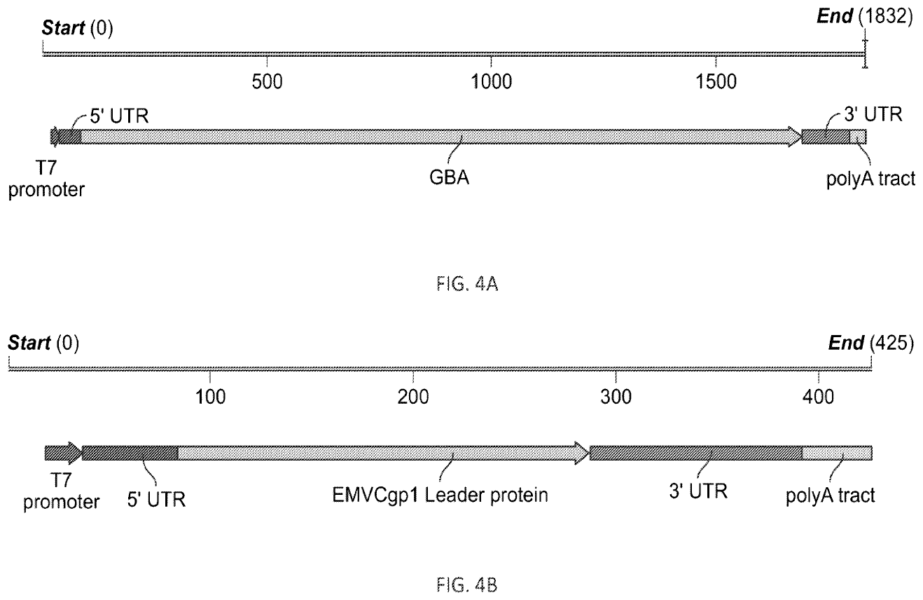

To test whether GBA as a secreted protein is expressed in HEK293T, Vero E6, C2C12 and/or BJ Fibroblast cells the following experiment is performed. HEK293T, Vero E6, C2C12 and/or BJ Fibroblast cells are transfected with RNA expressing GBA alone as a control (FIG. 4A) and in parallel co-transfected with two RNA constructs, one expressing GBA protein and the other expressing the NCT inhibitory protein (FIGS. 4A and 4B) from IVT reactions, as described next. On Day 0, HEK293T, Vero E6, C2C12 and/or BJ Fibroblast cells are seeded in 24-well cell culture microplates in 500 μl DMEM (HEK293T, Vero E6, C2C12) or 500 μl MEM (BJ Fibroblast) containing 10% FBS. On Day 1, the cells are transfected with respective RNA-Luc alone or in combination with RNA-L from IVT reactions using Lipofectamine™ MessengerMAX™ Transfection Reagent (Invitrogen) according to manufacturer's instructions. On following days, the transfected cells and cell culture supernatants are assayed for the activity of GBA.

Additionally, to measure the Interferon sensitivity with and without the NCT inhibitor protein, cells are treated with IFN-beta 6-24 h post transfection. For this mouse (for C2C12 cells) or human (for HEK293T, Vero E6, BJ Fibroblast cells) recombinant IFN-beta (Fisher Scientific) is 5-fold serially diluted and added to cells 6 hours or 24 hours post transfection with RNA encoding GBA protein alone or in combination with the NCT inhibitory protein. The ratio of GBA expression is normalized to an untreated sample. The EC50 value of IFN sensitivity is calculated in Prism (GraphPad) using the log (inhibitor) vs. response—variable slope four parameters nonlinear regression.

For interferon producing cell lines (C2C12, BJ Fibroblast) the secretion of interferons as marker for inflammation is measured. For this cell supernatants are collected 24-72 hours post transfection and cleared by centrifuged at 500 g for 5 mins. Clarified supernatants are then analysed for the presence of IFN-beta by ELISA (R&D Systems) according to the manufacturer's instructions.

For the GBA activity assay, the cells are seeded and transfected with respective RNA as described above. On Day 3, cell culture supernatant is collected, cleared from cell debris by centrifugation, and kept for assaying secreted GBA activity. Adherent cells are detached from the plate using 500 μl PBS and pelleted by centrifugation. The cell pellet is lysed using 1×GBA Assay Buffer (0.1 M sodium citrate, 0.2 M sodium phosphate, 0.25% Triton X-100, 0.25% sodium taurocholate, 1.25 mM EDTA, 5 mM DTT), pre-equilibrated to 37° C. prior for lysis. The protein concentration of the cell lysate is determined using the Pierce A660 protein assay per the manufacturer's instructions, after which the cell lysate was diluted to the final protein concentration of 125 μg/ml using 1×GBA Assay Buffer. 40 μl of pre-diluted cell lysate was pipetted to individual wells of a clear 96-well plate in duplicate. 20 μl of cell culture supernatants, as collected earlier, were added to individual wells of a clear 96-well plate in duplicate, and 20 μl of 2×GBA Assay Buffer was added to each well that contained cell culture supernatant. Thereafter, 20 μl of Assay Substrate (6 mM 4-MU-beta-D-glucopuranoside prepared in 1×GBA Assay Buffer) is added to each well that contained cell lysate or cell culture supernatant. In adjacent wells, a calibration curve using 4-methyl-umbelliferone is prepared in 1×GBA Assay Buffer. The samples are incubated in the presence of Assay Substrate at 37° C. for 30 minutes-4 hours, followed by the addition of 100 μl of Stop Solution (0.5 M Glycine, 0.3 M NaOH at pH10). Thereafter, the fluorescence of each sample and standard is measured at Ex/Em wavelength of 360/445 nm using a Biotek Synergy LX plate reader. GBA activity in cell lysate samples is calculated using the following equation: Activity=[B/(T×V×P)]×D=pmol/min/mg=μU/mg, where B is converted 4-MU amount as calculated using the standard curve (pmol), T is the reaction time (min), V is the sample volume (ml), P is the initial protein sample concentration, and D is the sample dilution factor (if applicable). GBA activity in cell culture supernatants is calculated using the following equation: Activity=[B/(T×V)]×D=pmol/min/ml=μU/ml, where B is converted 4-MU amount as calculated using the standard curve (pmol), T is the reaction time (min), V is the sample volume (ml), and D is the sample dilution factor (if applicable).

### Example 5: In Vivo Expression of Luciferase from RNA

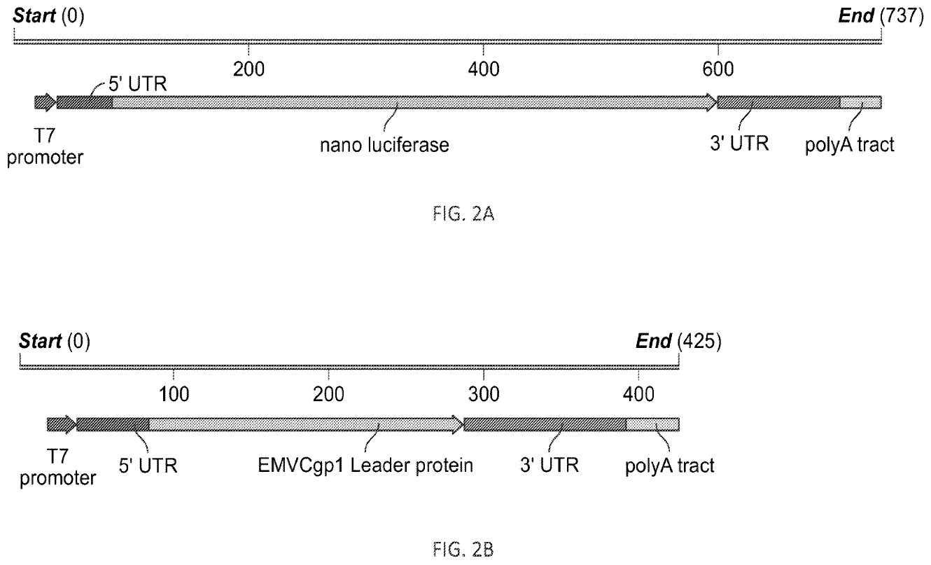

To evaluate the expression of nano luciferase in vivo, groups of 5 mice each of 6-8 weeks old female C57BL/6 mice are injected intramuscularly with RNA complexed with lipid nano-particle formulation in PBS (137 mM NaCl, 2.7 mM KCl, 10 mM Na2HPO4, 1.8 mM KH2PO4 pH 7.4), of (a) the RNA encoding luciferase shown in FIG. 2A or (b) the RNA encoding luciferase sequential with a NCT inhibitory protein. For baseline measurements, 4 mice are injected intramuscularly with PBS only. The volume of the dose is administered using a syringe/needle.

Animals are randomized in groups and bodyweight was recorded on day 1 and then bi-weekly until the end of the study. Adverse events (RM, SD, RD) are recorded according to good laboratory standards. Any individual animal with a single observation of >30% body weight loss or three consecutive measurements of >25% body weight loss was euthanized.

Whole blood is collected by submandibular vein and processed to collect serum for analysis. Blood was collected on Day 0 (prior to dosing), Day 3, Day 7, Day 14, Day 21, Day 28 and Day 42. Serum was analyzed for luciferase concentration by NanoLuc Assay.

For the NanoLuc assay 50 μl of diluted blood serum was loaded to opaque white 96-well microplates. 50 μl of fresh 1× Nano-Glo assay reagent in the respective assay buffer (Promega) was added to each well, incubated 2-5 minutes, followed by luminescence measurement using a Biotex Synergy LX plate reader.

### Example 6: Immunogenicity of Sars-CoV2 Spike from RNA In Vivo

To evaluate the expression of Sars-CoV2 Spike in vivo, groups of 5 mice each of 6-8 weeks old female C57BL/6 mice are used. The groups are injected intramuscularly (prime) on day 0 and boosted in the same manner on day 28 with RNA complexed with lipid nano-particle formulation in PBS (137 mM NaCl, 2.7 mM KCl, 10 mM Na2HPO4, 1.8 mM KH2PO4 pH 7.4), of (a) the RNA encoding Sars-CoV2 Spike shown in FIG. 3A or (b) the RNA encoding Sars-CoV2 Spike sequential with an NCT inhibitory protein. For baseline measurements, 4 mice are injected intradermally with PBS only. The volume of the dose was administered using a syringe/needle.

Animals are randomized in groups and bodyweight was recorded on day 1 and then bi-weekly until the end of the study. Adverse events (RM, SD, RD) were recorded according to good laboratory standards. Any individual animal with a single observation of >30% body weight loss or three consecutive measurements of >25% body weight loss was euthanized.

Whole blood is collected by submandibular vein and processed to collect serum for analysis. Blood was collected on Day 0 (prior to dosing), Day 3, Day 14, Day 28 and Day 42. Serum was analyzed for anti-Sars-CoV2 Spike antibodies (the elicited humoral immune response) by ELISA Assay enzyme linked immunosorbent assay (ELISA) is performed using purified SARS-COV-2 Spike RBD protein as coating material. High-binding 96-well plates were coated with 75 μl of a 2 ug/ml SARS-COV-2-Spike RBD (Creative Diagnostics® DAGC149 Recombinant SARS-COV-2 Spike Protein Receptor Binding Domain [His]) and plates are incubated over night at 4° C. After incubation, plates are washed twice with 0.05% Twen-20 in PBS and wells were blocked using EZ block (2 h at 37 C). Plates are washed twice with 0.05% Tween-20 in PBS and serum collected from the mice added to the wells (1:500 dilution for binding antibody detection, 1:100-1:7812500 for Endpoint Titer measurement). Serum is incubated for 1 h at RT before washing thrice with 0.05% Twen-20 in PBS and adding 75 μl of secondary antibody (Goat-Anti-rabbit, HRP-conjugate, 1:4,000 dilution) and incubating for 1 h at RT. Wells are thoroughly washed (5× with 0.05% Twen-20 in PBS) and binding was developed using 75 μl 3,3′,5,5′-tetramethylbenzidine (TMB) substrate (Surmodisc Inc TMB One Component HRP Microwell Substrate). The reaction is carried out for 30 minutes before stopping with 75 μl Stop Solution (Surmodisc Inc 450 NM LIQ STOP REAGENT) and the Absorbance at 450 nm is measured using a Biotex Synergy LX plate reader. Endpoint titers are calculated in Prism (GraphPad) using the log (inhibitor) vs. response-variable slope four parameters nonlinear regression from interpolated values from four-fold over background.

### Example 7: The Effect of L Protein Expression on Reporter Gene Expression when Cells are Exogenously Stimulated with Poly I:C or IFN-Beta

Poly I:C and IFN-beta, activators or innate immune signaling pathways, reduce reporter gene expression in a dose dependent manner. The EMCV L protein as an NCT inhibitor protein, protects reporter gene expression from being downregulated.

mRNA once inside the cell may encounter host or administration dependent induced inflammation which can inhibit the expression and subsequently the therapeutic effect of an encoded gene of interest (GOI). The effect of the protein L was evaluated when added in trans to human skin fibroblast cells transfected with mRNA encoding a reporter GOI (Firefly luciferase) and stimulated with Poly I:C, a synthetic double stranded RNA analogue acting as a potent immunostimulant.

Human skin fibroblast cells (BJ cells, ATCC) were grown as described in Example 1. On day 0, the BJ cells were seeded into 96-well cell culture plates at 1e4 cells/well in complete DMEM (10% heat inactivated FBS+1× Antibiotic-Antimycotic). Cells were incubated overnight at 37° C. and 5% CO2. On day 1, the cells were transfected with the indicated RNA constructs using the Mirus TransIT mRNA Transfection Kit (Mirus) as described in Example 1. Within 5 minutes from adding the transfection complexes to the cells, Poly I:C or IFN-beta was added to the cells at indicated final concentrations. The reporter mRNA (Trilink), encoding for Firefly luciferase, featured CleanCap mRNA cap structure, and 100% of uridines in its sequence had been substituted with 5-methoxyuridines. This type of modified mRNA was used because it is a weak activator of innate immune signaling pathways in recipient cells, thus allowing us to focus predominantly on the effects of L protein mRNA in the context of exogenously applied inflammatory stimulus. L protein-encoding mRNA and nLuc filler mRNA were in vitro transcribed using the HiScribe™ T7 mRNA Kit with CleanCap Reagent AG (NEB) according to manufacturer's instructions, as described in Example 1, both containing 100% N1-Methyl-pseudouridines to reduce their capacity to natively activate pro-inflammatory cell signaling pathways. For the transfections, 10 ng of the reporter mRNA was mixed with an indicated amount of L protein-coding mRNA and the filler nLuc mRNA to make sure that all transfections were carried out using equal amount of mRNA. 2 days after transfection, Firefly luciferase expression from the reporter gene was measured using the Bio-Glo Luciferase Assay (Promega) as per manufacturer's instructions.

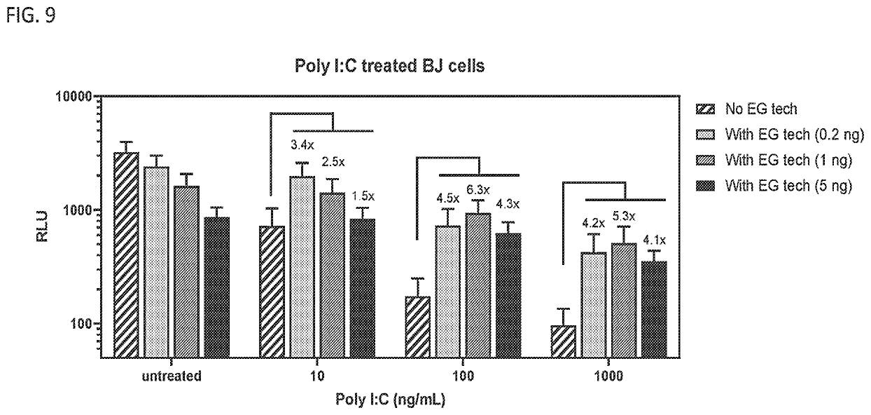

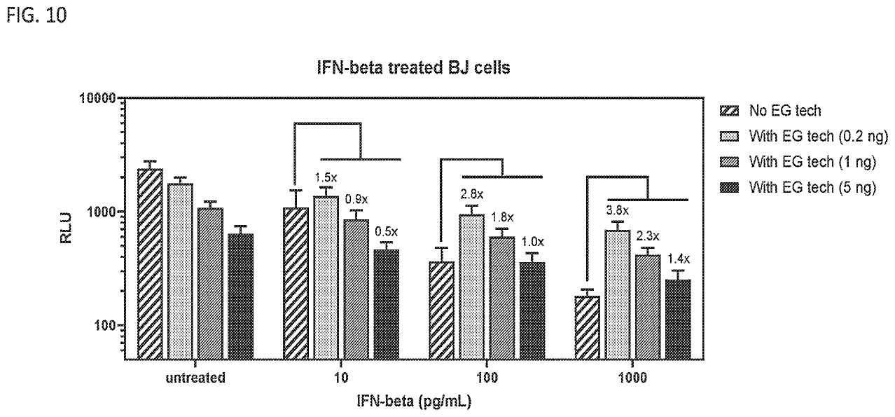

As shown in FIGS. 9 and 10, Poly I:C and IFN-beta, respectively, as strong activators of innate immune cell signaling pathways, inhibit Firefly luciferase expression in a dose dependent manner (diagonally striped bars). However, in the presence of the L NCT inhibitor protein-encoding mRNA (gray and black bars), the effects of Poly I:C and IFN-beta (FIG. 9 and FIG. 10, respectively) to reduce Firefly luciferase is greatly reduced. Thus, L NCT inhibitor protein protects GOI expression in the presence of excessive and inhibitory levels of inflammation modelled by addition of Poly I:C and IFN-beta. As expected from its anticipated mechanism of action to inhibit nucleocytoplasmic transport, the NCT inhibitor protein L encoding mRNA inhibited overall reporter gene expression to a certain extent when no pro-inflammatory signal was present because inhibition of that pathway has been linked to downregulation of protein translation to some extent in general. Notably, however, in the presence of Poly I:C and IFN-beta, L NCT inhibitor protein-encoding mRNA helped to rescue reporter gene expression (FIG. 9 and FIG. 10). As a general observation, the fold increase in the rescue of reporter gene expression was greater at higher concentrations of pro-inflammatory stimuli, both in case of Poly I:C and IFN-beta. Interestingly, in the case of Poly I:C as the stimulus, 1 ng of L NCT inhibitor mRNA displayed the strongest effects in the rescue of reporter gene expression, whereas in case of IFN-beta, the strongest effects were observed at 0.2 ng of L NCT inhibitor mRNA. This suggests that depending on the main source and nature of the pro-inflammatory stimulus, different amounts of the L NCT inhibitor mRNA might be needed (and, more importantly, can be used) to rescue gene-of-interest expression induced by activated innate immune cell signaling pathways as needed.

### Example 8: The Effect of L Protein on Reporter Gene Expression when Cells are Endogenously Stimulated with an Intracellularly Delivered Pro-Inflammatory mRNA Transgene (i.e., mRNA that Contains Natural Uridines)

In vitro transcribed mRNA containing natural nucleosides is known to activate immune signaling pathways, which in turn can lead to the downregulation of intracellularly delivered mRNA expression (as shown in Example 7). mRNA containing N1-methyl-pseudouridines is known to induce immune activation at low levels, which is generally not related with intracellularly delivered mRNA expression downregulation. Consistent with the latter observations, L NCT inhibitor protein-encoding mRNA enhances GOI expression from strongly pro-inflammatory mRNA containing natural uridine nucleosides, while not form the non-inflammatory mRNA containing N1-methyl-pseudouridines.

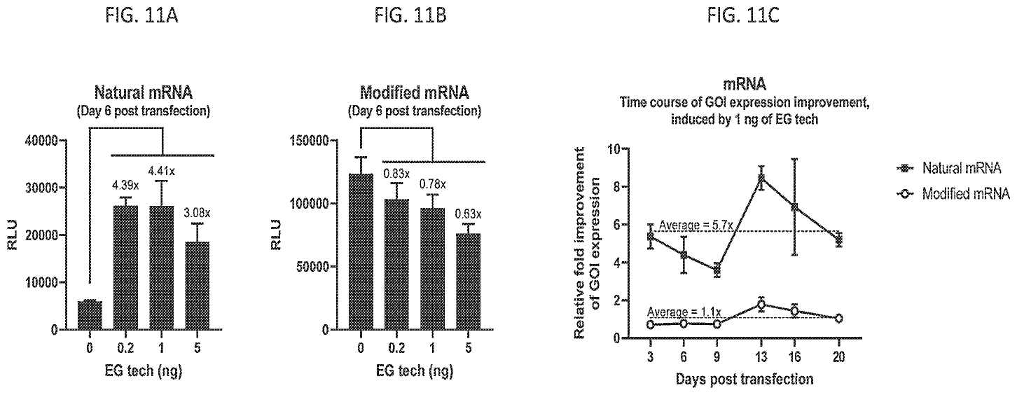

L NCT inhibitor protein-coding mRNA was produced in house using the HiScribe T7 mRNA Kit with CleanCap Reagent AG Kit (NEB) with 100% ml-pseudouridine (Trilink), and polyA-tailing (NEB) according to the manufacturer's instructions. Nano luciferase expressing mRNA was synthesized in the same manner, but with or without substitution of synthetic nucleosides, as indicated in the FIGS. 11A-11C. mRNA synthesis was conducted as described in Example 1 above.

For the transfections, 10 ng of the reporter mRNA was mixed with an indicated amount of L NCT inhibitor protein-coding mRNA and an irrelevant filler mRNA to make sure that all transfections were carried out using equal amount of mRNA. 2 days after transfection, Nano-luciferase expression from the reporter gene was measured using the Nano-Glo Luciferase Assay (Promega) as per manufacturer's instructions.

First, it was observed that on Day 6 after mRNA transfection, L NCT inhibitor protein-encoding mRNA increased the expression of the reporter gene 3.1-4.4-fold, depending on the amount of L NCT inhibitor protein-encoding mRNA co-transfected with the reporter gene, but only when the reporter gene-encoding mRNA was proinflammatory (i.e., containing only natural nucleosides) (FIG. 11A). When pseudo-uridine containing reporter gene-encoding non-inflammatory mRNA was transfected to the cells, L NCT inhibitor protein did not increase transgene expression, as expected (FIG. 11B).

To further analyze the observed phenomenon of increasing the gene expression from inherently inflammatory mRNA, a time course experiment was conducted. BJ cells were co-transfected with pro-inflammatory natural mRNA or non-inflammatory N1-Methyl-pseudouridine-modified mRNA as above and 1 ng of L NCT inhibitor protein-encoding mRNA, and measured luciferase expression on Day 3, 6, 9, 13, 16 and 20 post transfections. Similarly to the earlier mentioned results, transgene expression was improved at all time points in case when the reporter gene-encoding mRNA was proinflammatory (i.e., containing only natural nucleosides), on an average of 5.7-fold across all time points (FIG. 11C). However, when transfecting BJ cells with the non-inflammatory pseudouridine-containing reporter mRNA, L NCT inhibitor protein affected the expression from the reporter mRNA only marginally at all time points (FIG. 11C), which is in line with the results reported earlier.

These data demonstrate advantages of using L NCT inhibitor protein-encoding mRNA to increase gene expression in applications that require the use of natural or partially natural mRNA molecules.

### Example 9: The Effect of L Protein on Reporter Gene Expression when Cells are Endogenously Stimulated with an Intracellularly Delivered Pro-Inflammatory Self-Amplifying RNA (saRNA) Transgene

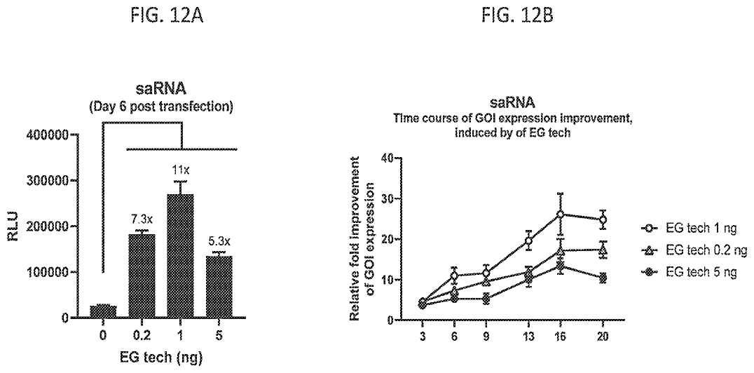

mRNA once inside the cell may encounter host or administration dependent induced inflammation which can inhibit the expression and subsequently the therapeutic effect of an encoded gene of interest (GOI). One therapeutic modality that induces excess inflammation is self-amplifying mRNA (saRNA). The role of the EGT-404 on reporter gene expression encoded on an saRNA cassette derived from Venezuelan equine encephalitis virus (VEEV) in human fibroblasts was evaluated. When delivered from an independent mRNA molecule, L NCT inhibitor protein-encoding mRNA enhanced saRNA in a dose dependent manner at all time points measured (FIG. 12A and FIG. 12B).

Human skin fibroblast cells (BJ cells) were cultured and transfected as in Examples 7 and 8. L NCT inhibitor protein-encoding mRNA was produced using the HiScribe T7 mRNA Kit with CleanCap Reagent AG Kit (NEB) with 100% ml-pseudouridine (Trilink), and polyA-tailing (NEB) according to the manufacturer's instructions. The reporter gene (nano-luciferase) encoding VEEV saRNA replicon was purchased from Aldevron. All transfections were made using 100 ng of saRNA, and the indicated amount of L NCT inhibitor protein-encoding mRNA. An irrelevant mRNA was added to the mix as a filler mRNA in the appropriate volume to maintain constant total mass of mRNA. At indicated time points, the expression of the nano-luciferase was measured using the Nano-Glo Luciferase Assay System (Promega) as per manufacturer's instructions.

saRNA-encoded nano-luciferase expression was first analyzed on Day 6 post transfection. The L NCT inhibitor protein enhanced luciferase expression in a concentration dependent manner, up to ˜11-fold as compared to the condition where L NCT inhibitor protein-encoding mRNA was not used (FIG. 12A), the optimal amount of L NCT inhibitor mRNA being I ng. The relative effect of L NCT inhibitor mRNA to improve saRNA reporter gene expression was increased over time, up to 26-fold on Day 16 and 20 post transfections, as compared to the condition where L NCT inhibitor protein-encoding mRNA was not used (FIG. 12B). At each time point, the optimal L NCT inhibitor mRNA amount was 1 ng, similarly to what was observed in Day 6. These results suggest that L NCT inhibitor mRNA helps to increase transgene expression level and expression duration when using self-amplifying RNA, which during its self-replication process is a known strong inducer of innate immune signaling pathways in cells. This demonstrates and is in line with the results of Examples 8-8, that L NCT inhibitor protein-encoding mRNA increased transgene expression when innate immune signaling pathways were activated by exogeneous or endogenous stimuli.

### Example 10: Alternative Mechanisms of Nucleocytoplasmic Transport Inhibition to Enhance Protein Expression

Nucleocytoplasmic transport (NCT) inhibition can also be achieved via alternative mechanisms than as described in Examples 1-9, e.g., with non-viral proteins which bind to NCT pathway proteins, or with RNAi to knock down the expression of NCT pathway genes. For example, overexpressing dominant-negative mutants of NCT pathway proteins, e.g., Ran Q69L mutant or MxB K131A protein can block NCT. Similarly, myristoylated peptides derived from the ERK and SMAD proteins inhibit Nup93-dependent NCT of transcription factors and RNA. In this example, methods for knock down of NCT proteins, e.g., Ran, NTF2, MxB, Xpo7, and Nup93, with siRNA are described.

As described in Example 7, Poly I:C and IFN-beta are known activators of innate immune signaling pathways and reduce the expression of delivered mRNA molecules. Inhibition of these signaling pathways with an NCT inhibitor protein allowed for increased target protein expression. As described herein, siRNA inhibition of NCT pathway proteins can also inhibit Poly I:C and IFN-beta signaling to increase target protein expression.

Methods for siRNA Knock Down of NCT Inhibitor Proteins

Briefly, on Day 0, BJ cells are seeded into 96-well cell culture plates at 1E4 cells/well in complete DMEM (i.e., containing 10% heat-inactivated FBS+1× Antibiotic-Antimycotic solution). The cells are incubated overnight at 37° C. and 5% CO2. On Day 1, the cells are co-transfected with a reporter mRNA and NCT inhibitory RNA. Transfections are conducted using the Mirus TransIT mRNA Transfection Kit (Mirus) as described in Example 1, using 10 ng of the reporter mRNA and an appropriate amount of NCT inhibitory RNA. Within 5 minutes of adding the transfection complexes to the cells, Poly I:C or IFN-beta is added to the cells at indicated final concentrations.

The reporter mRNA encodes for Firefly luciferase, featuring CleanCap mRNA cap structure, and 100% of the uridines in the sequence are substituted with N1-methylpseudouridine. Luciferase mRNA is co-transfected with any of the following NCT inhibitory RNAs (a) mRNA encoding for anti-Xpo7 nanobody, (b) mRNA encoding for anti-NFT2 antibody, (c) mRNA encoding for Ran Q69L mutant protein, (d) mRNA encoding for MxB K131A mutant protein, (c) mRNA encoding for myristoylated ERK-derived phosphomimetic peptide, (c) mRNA encoding for myristoylated SMAD-derived phosphomimetic peptide, (f) siRNA targeted to Ran mRNA, (g) siRNA targeted to NTF2 mRNA, (h) siRNA targeted to MxB mRNA, (i) siRNA targeted to Xpo7 mRNA, or (j) siRNA targeted to Nup93 mRNA. NCT inhibitory mRNAs are in vitro transcribed using the HiScribe™ T7 mRNA Kit with CleanCap Reagent AG (NEB) according to the manufacturer's instructions, as described in Example 1, containing 100% N1-Methyl-pseudouridines. NCT inhibitory siRNAs are commercially obtained as chemically synthesized sequences (IDT Technologies).

On Day 3 (i.e., 48 hours after the transfection), Firefly luciferase expression from the reporter gene is measured using the Bio-Glo Luciferase Assay (Promega) as per the manufacturer's instructions. The effect of NCT inhibitory RNA on the enhancement of Luciferase expression at any given Poly I:C and IFN-beta concentration by calculating the fold difference of Luciferase expression in the presence and the absence of the respective NCT inhibitory RNA.

### Example 11: Mutant Forms of L Protein Regulate Target Protein Expression Levels

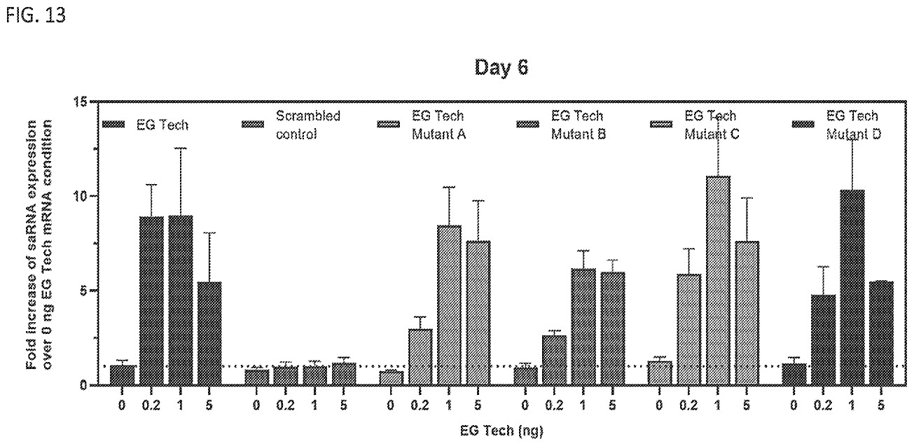

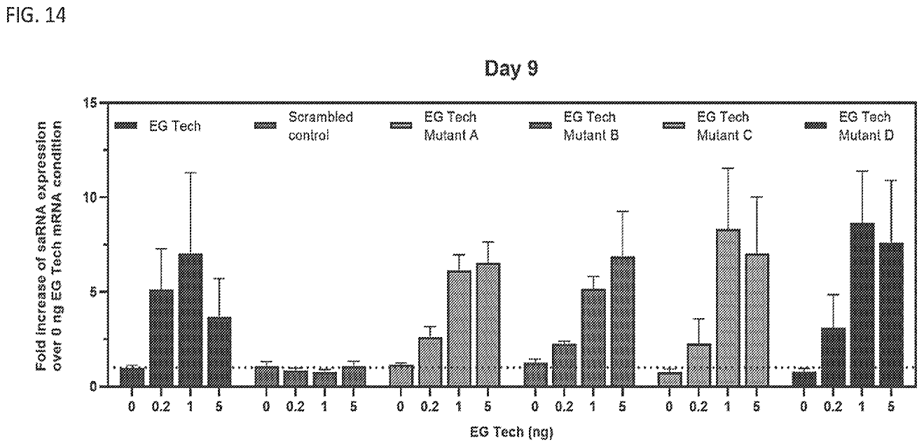

Co-expression of an L protein mRNA and saRNA expression of the reporter gene luciferase was performed as described in Example 9. Here, mutated forms of the EMCV L protein (SEQ ID NOS: 65, 66, 67, 68) differentially regulated saRNA based reporter gene expression. The comparable wild type EMCV L protein sequence is SEQ ID NO: 69. Reporter gene expression was also regulated by the concentration of transfected L protein mutants (FIGS. 13 and 14).

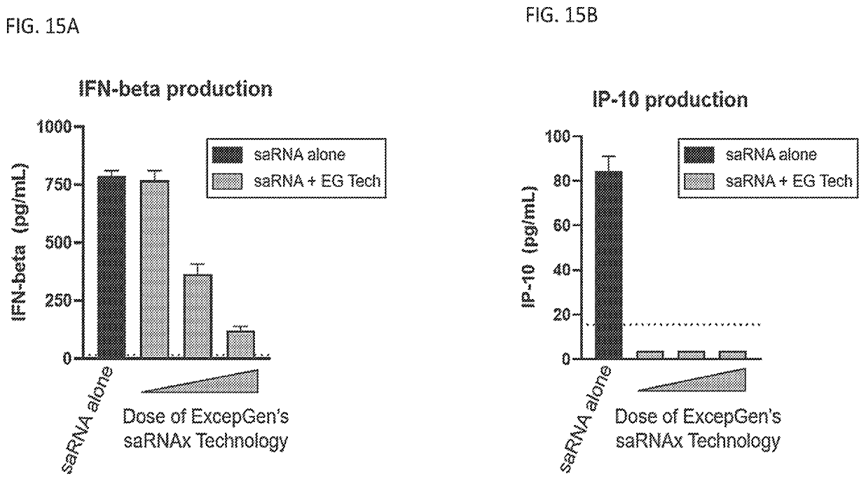

The same system, L protein mRNA and saRNA-based expression of reporter gene luciferase, was used to test the expression of associated inflammation mediators. Expression of IP-10 (FIG. 15B) and IFN-beta (FIG. 15A) both decreased with the introduction of the aforementioned L protein saRNA system, as measured 6 days post transfection. Data shown are from N=3 experiments.

### Example 12. The Effect of L Protein on Reporter Gene Expression In Vivo when the Reporter Gene is Expressed Using Self-Amplifying RNA (saRNA)

To demonstrate the beneficial effects of L protein on saRNA-encoded reporter gene expression in vivo, the following experiment was performed. L protein encoding mRNA (or the respective scrambled negative control sequence) was produced in-house, as described in Example 9. The saRNA vector expressing Nanoluciferase was purchased from Aldevron, as described in Example 9. Nanoluciferase saRNA and L protein mRNA (or Nanoluciferase saRNA and the respective scrambled negative control mRNA) were mixed, and then formulated into lipid nanoparticles (LNPs) containing SM-102, 1,2-DSPC, cholesterol and DMG-PEG (2000) lipids (SM-102 LNPs) and stored in 20 mM Tris pH 7.5 8.7% sucrose buffer. C57BL6 mice (female and 10-12 weeks old) were treated with SM-102 LNPs, with total doses of 2 μg/mouse, and N=4 mice per group, by intramuscular injection. On indicated days, Nanoluciferase expression was measured by in vivo imaging using IVIS as follows. Nanolucifase substrate fluorofurimazine (FFz) was prepared per manufacturer's instructions (Promega) and administered to mice by intraperitoneal (i.p.) injections using 50 μl/mouse. IVIS imaging was performed 10 min after FFz i.p. injection.

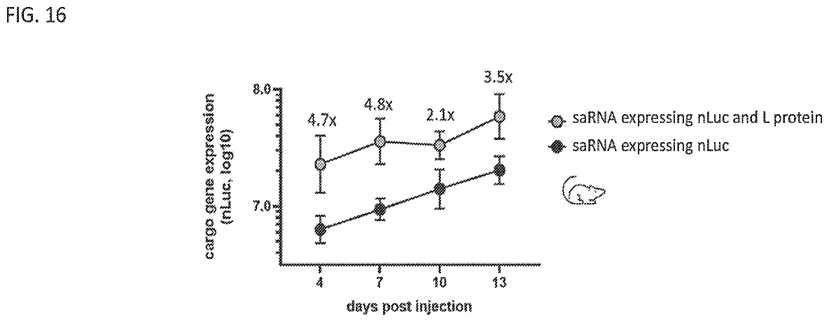

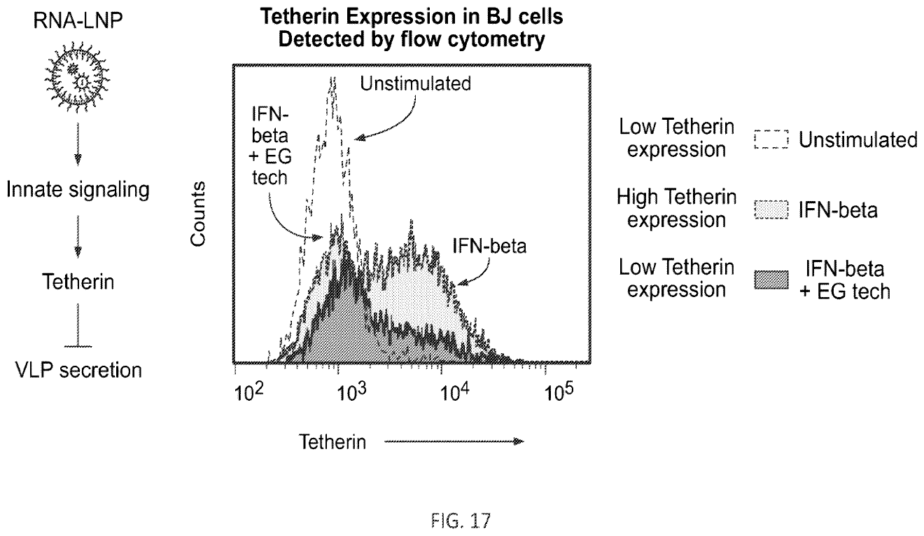

FIG. 16 shows the effects of L protein expression on gene of interest expression with self-amplifying RNA (saRNA) in C57BL6 mice, transfected with lipid nanoparticles carrying the L protein and saRNA system. Mice were injected with 2 μg of SM-102 LNPs, and 4 mice were used per experimental group. As shown, saRNA based luciferase expression in vivo, was from between 2-4 fold higher in the presence of L protein mRNA.

### Example 13. The Effect of mRNA Expression of the L Protein on mRNA Expression of Therapeutically Relevant Proteins

In this example the effect of L protein (EG Tech) mRNA on the expression of therapeutically relevant gene mRNA vectors, when co-delivered to target cells, was investigated. The following therapeutically relevant gene mRNA vectors were studied: G6PC1 and SERPINA1. The G6PC1 gene is mutated in Glycogen Storage Disease Type I (GSDI), also known as the von Gierke Disease, which results in the body's ability to break down glycogen, which leads to hypoglycemia, hepatomegaly and nephromegaly. Expressing functional G6PC1 in a gene therapy setting can have therapeutic benefits in GSDI. SERPINA1 mutations lead to the development of Alpha-1 Antitrypsin Deficiency (A1AD) disease, which leads to lung and liver damage of affected individuals. Expressing functional SERPINA1 in a gene therapy setting can have therapeutic benefits in A1AD.

To be able to evaluate the expression G6PC1 and SERPINA1, mRNA vectors were designed in which the therapeutically relevant genes of interest (GOIs) were fused to the NanoLuc reporter protein via a linker peptide (FIGS. 18A and 18B). The coding sequences of these GOIs were flanked by hBG 5′ UTR and hBG 3′UTR. mRNA expression vectors contained the Cap1 mRNA cap structure, N1-methylpseudouridine modifications and a 120-nt polyA tail. GOI mRNA expression vectors were produced using in vitro transcription (IVT) by a commercial 3rd party provider. L protein (EG Tech) mRNA also contained the Cap1 mRNA cap structure and N1-methylpseudouridine modifications and were produced using in vitro transcription (IVT) by a commercial 3rd party provider.

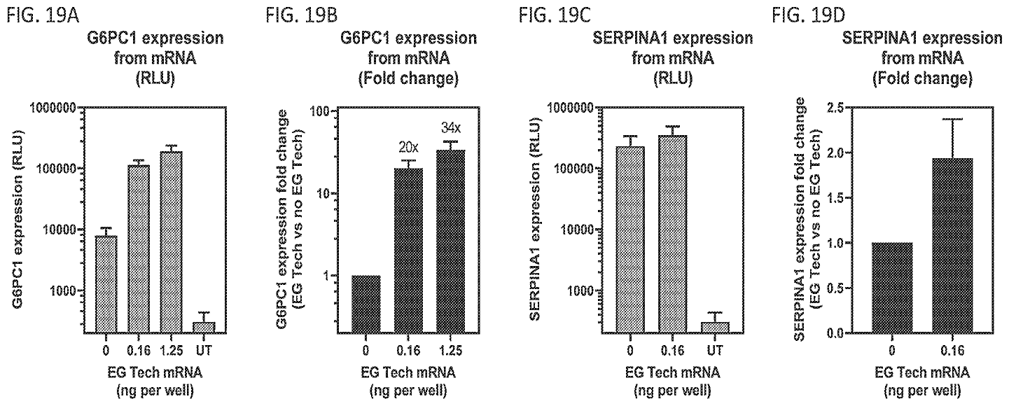

Mouse fibroblast NIH/3T3 cells were obtained from ATCC and grown according to the cell bank's instructions. Briefly, the cells were grown at 5% CO2 37° C. humidified environment using high glucose and GlutaMAX supplemented Dubelcco's Modified Eagle Medium (DMEM), which contained 10% fetal bovine serum (FBS) and 1× antimycotic/antibiotic solution. On Day 0, NIH/3T3 cells were seeded onto 96-well plates. On Day 1, the cells were transfected with 10 ng GOI mRNA per well, or with a mixture of 10 ng GOI mRNA and 0.16 ng EG Tech mRNA per well, or with a mixture of 10 ng GOI mRNA and 1.25 ng L protein (EG Tech) mRNA per well, using the MIRUS TransIT mRNA Transfection Kit according to the manufacturer's instructions. On Day 3, i.e., 48 hours after transfections, cell supernatants were removed, and the expression of therapeutically relevant GOIs (G6PC1 and SERPINA1) were determined in cell lysates by measuring the fused NanoLuc reporter activity using the Promega Nano-Glo Luciferase Assay System according to the manufacturer's instructions. Each experiment was performed using three technical replicates. A total of five independent experiments (biological replicates) were performed. The results are presented in FIGS. 19A-19D.

The expression level of G6PC1 mRNA vector in NIH/3T3 cells was moderate (<10,000 RLU) in the absence of L protein (EG Tech) mRNA; however, when co-transfecting G6PC1 mRNA with L protein (EG Tech) mRNA, the expression of G6PC1 is increased (>100,000 RLU), FIG. 19A. This corresponded to 20-fold or 34-fold increase in G6PC1 expression, depending on the amount L protein (EG Tech) used for the co-transfection, FIG. 19B. Even though the basal expression level of the SERPINA1 mRNA vector was relatively high in the absence of L protein (EG Tech) mRNA (>100,000 RLU), FIG. 19C, co-transfection of L protein (EG Tech) mRNA led to a further ˜2-fold increase in SERPINA1 expression (FIG. 19D). These results indicated that mRNA expression of the L protein can have a significant benefit in increasing gene expression of therapeutically relevant GOI mRNA vectors, expanding the applicability area of the L protein into gene replacement therapies and gene therapies in general.

### Example 14: The Effect of mRNA Expression of the L Protein on Therapeutically Relevant Plasmid DNA Expression of Target Proteins

In this example the effect of L protein (EG Tech) mRNA on the expression of a therapeutically relevant gene in a pDNA vector, when co-delivered to target cells, was evaluated. The therapeutically relevant gene, GBA (lysosomal acid glucosylceramidase), is important for turnover of cellular membranes, degradation of complex lipids and cholesterol processing. Mutations in that gene can lead to the development of a lysosomal storage disorder called Gaucher's Disease in which the accumulation of glucoserebroside lipid accumulates in abnormal levels in different organs, which can lead to a wide range of dysfunctions. Gene replacement therapies using a fully functional GBA gene can have a therapeutic benefit.

To evaluate the capacity of L protein (EG Tech) mRNA to express the GBA protein from a plasmid DNA (pDNA) expression vector a pDNA expression vector was first designed, where GBA was fused to the NanoLuc reporter protein via a peptide linker, whereas the expression of GBA-NanoLuc fusion protein was driven by the CMV promoter (FIG. 20A). The GBA pDNA was transfected to cells alongside L protein (EG Tech) mRNA as follows.

Mouse fibroblast NIH/3T3 cells were obtained from ATCC and grown according to the cell bank's instructions. Briefly, the cells were grown at 5% CO2 37° C. humidified environment using high glucose and GlutaMAX supplemented Dubelcco's Modified Eagle Medium (DMEM), which contained 10% fetal bovine serum (FBS) and 1× antimycotic/antibiotic solution. On Day 0, NIH/3T3 cells were seeded onto 96-well plates. On Day 1, the cells were transfected with 0.3 ng L protein (EG Tech) mRNA per well using the MIRUS TransIT mRNA Transfection Kit according to the manufacturer's instructions. Immediately after adding the L protein (EG Tech) mRNA complexes to the cells, the cells were further transfected with GBA-NanoLuc pDNA (50 ng per well) using the MIRUS TransIT-Lenti transfection reagent according to the manufacturer's instructions.

Because Gaucher Disease is related to elevated inflammation in affected tissues, and since elevated inflammation is known to suppress expression from pDNA, in some settings IFN-beta (1000 pg/ml) or Poly I:C (1000 ng/ml) were added to the cells 4 hours after transfection to model the inflammatory environment and to study the impact of L protein (EG Tech) mRNA in GOI expression from a pDNA vector in those settings.

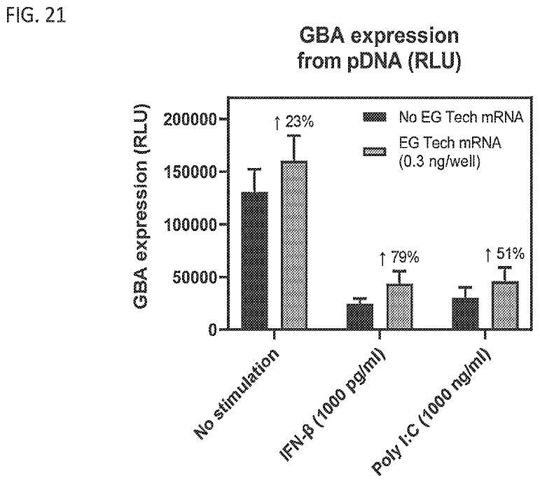

On Day 3, i.e., 48 hours after transfections, cell supernatants were removed, and the expression of the GBA protein was determined in cell lysates by measuring the fused NanoLuc reporter activity using the Promega Nano-Glo Luciferase Assay System according to the manufacturer's instructions. The results are presented in FIG. 21.

It was observed that the expression of GBA from a pDNA vector in non-stimulated cells was increased by 23% when 0.3 ng per well L protein (EG Tech) mRNA was used FIG. 21, left bars). The stimulation of cells with pro-inflammatory model molecules IFN-beta and Poly I:C led to approx. 80% reduction in GBA expression. Notably, in IFN-beta and Poly I:C stimulated cells, L protein (EG Tech) mRNA was able to rescue GBA expression from a pDNA vector by 79% and 51%, respectively (FIG. 21, middle bars and right bars). These results demonstrated that L protein (EG Tech) mRNA can enhance gene expression from pDNA vectors both in baseline conditions and in a strongly pro-inflammatory environment as well, which is particularly important for diseases with a string inflammatory component.

### Example 15: The Effect of mRNA Expression of the L Protein on Plasmid DNA Induced Cytokine Induction

The delivery of cytosolic plasmid DNA (pDNA) delivery can lead to the activation of cellular response pathways (e.g., the cGAS-STING and other pathways) that in turn can lead to the activation of excessive inflammatory pathways in the cells, resulting in lowered GOI expression and/or the induction of excessive inflammation in transduced cells. The latter can limit the applicability of therapeutic application of DNA vectors as excessive inflammation can be associated with unfavorable reactogenicity and other side effect profiles. Thus, in this example, the effect of L protein (EG Tech) mRNA to suppress the activation of pro-inflammatory mediators, induced by intracellularly delivered pDNA with or without additional pro-inflammatory stimuli was investigated.

GBA-expressing pDNA (as used earlier and depicted in (FIG. 20A)) was transfected to cells alongside L protein (EG Tech) mRNA as follows. Mouse fibroblast NIH/3T3 cells were obtained from ATCC and grown according to the cell bank's instructions. Briefly, the cells were grown at 5% CO2 37° C. humidified environment using high glucose and GlutaMAX supplemented Dubelcco's Modified Eagle Medium (DMEM), which contained 10% fetal bovine serum (FBS) and 1× antimycotic/antibiotic solution. On Day 0, NIH/3T3 cells were seeded onto 96-well plates. On Day 1, the cells were transfected with 0.3 ng or 1.25 ng L protein (EG Tech) mRNA per well using the MIRUS TransIT mRNA Transfection Kit according to the manufacturer's instructions. Immediately after adding the L protein (EG Tech) mRNA complexes to the cells, the cells were further transfected with GBA-NanoLuc pDNA (50 ng per well) using the MIRUS TransIT-Lenti transfection reagent according to the manufacturer's instructions. On Day 3, i.e., 48 hours after transfections, cell supernatants were removed, cleared from cell debris by centrifugation, and analyzed for the secretion of CCL2, an inflammatory molecule that is induced by cytosolic double stranded DNA via the cGAS-STING pathway, using the Biolegend LegendPlex Mouse Anti-Virus Response Panel according to the manufacturer's instructions.

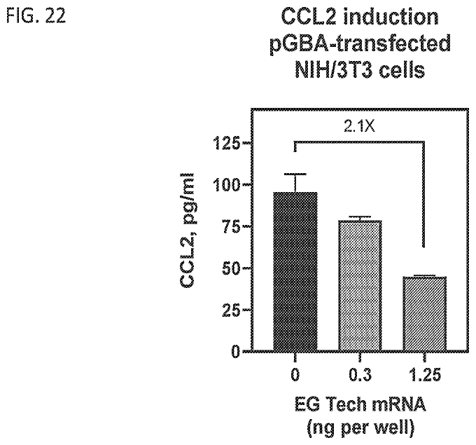

The induction of CCL2 in GBA-expressing pDNA-transfected cells was reduced when the pDNA was transfected alongside L protein (EG Tech) mRNA (FIG. 22). These results demonstrated that L protein (EG Tech) mRNA can reduce the activation of excessive inflammatory pathways in pDNA transfected cells, showing further promise in advancing pDNA technology applicability in vivo.

### Example 16: The Effect of mRNA Expression of the L Protein on Induced Inflammation in Plasmid DNA Transfected Cells

In this example the analysis of the effect of L protein (EG Tech) mRNA was expanded to help to suppress the induction of excessive inflammation in pDNA transfected cells. In addition to the GBA-expressing pDNA, an additional therapeutically relevant pDNA expression vector was designed where the NanoLuc reporter protein sequence was fused with the coding sequence of erythropoietin (EPO), downstream of the CMV promoter (FIG. 20B).

NIH/3T3 cells were grown and transfected with L protein (EG Tech) mRNA and GBA and EPO encoding pDNA as in the previous example, but to study the effect of L protein (EG Tech) mRNA to suppress pDNA-induced inflammation in even higher pro-inflammatory conditions, the cells were further stimulated with 1000 ng/ml Poly I:C. This experimental setup allowed us to study the effect of L protein (EG Tech) mRNA in the suppression of excessive pro-inflammatory molecules when the inflammatory signaling is induced by pDNA (e.g., the cGAS-STING pathway) and other orthogonal pathways. 48 hours after transfections, cell supernatants were removed, cleared from cell debris by centrifugation, and analyzed for the secretion of CCL2, CCL5, CXCL10, IFN-alpha and IFN-beta, using the Biolegend LegendPlex Mouse Anti-Virus Response Panel according to the manufacturer's instructions.

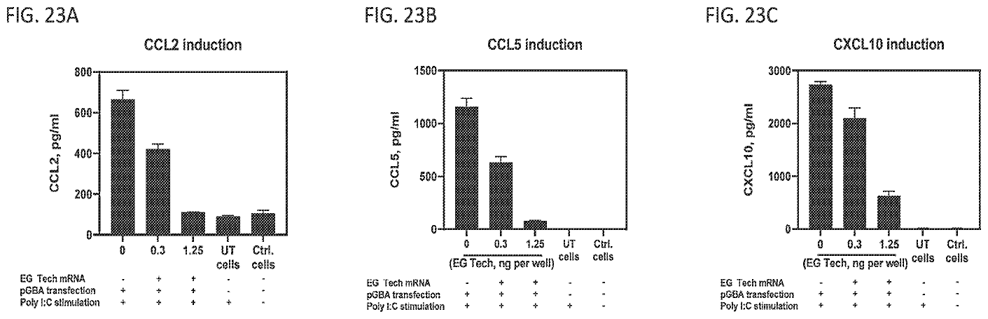

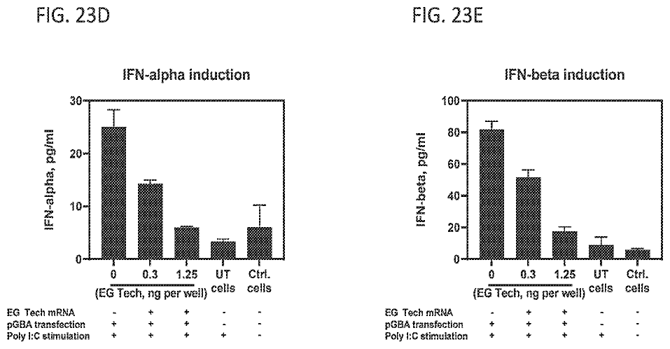

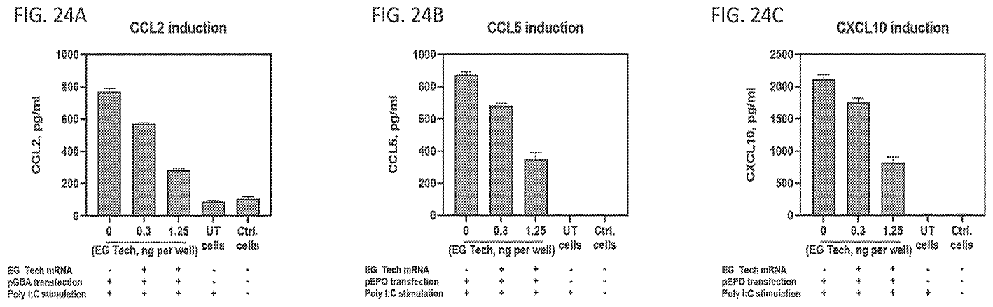

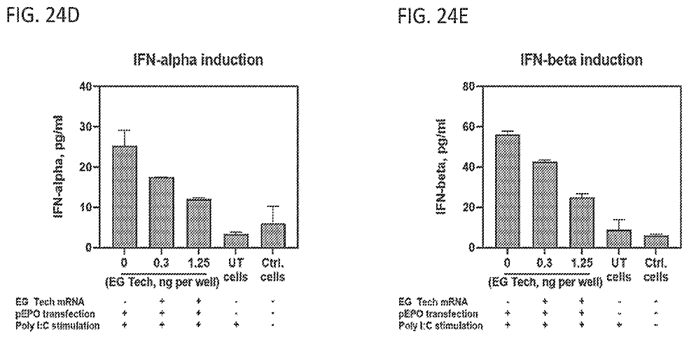

It was observed that the transfection of 3T3 with both the GBA-encoding pDNA (FIGS. 23A-23E) and EPO-encoding pDNA (FIGS. 24A-24E) and additionally stimulating the cells with Poly I:C induced a high level of secretion of inflammation related molecules CCL2, CCL5, CXCL5, IFN-alpha and IFN-beta. The additive effects of pDNA-induced and Poly I:C induced CCL2 were clearly evident when comparing the results with FIG. 22. Importantly, in the presence of L protein (EG Tech) mRNA, the levels of CCL2, CCL5, CXCL5, IFN-alpha and IFN-beta were reduced to the baseline levels of untransfected and Poly I:C non-stimulated cells. These results further underscore the desirability of using L protein (EG Tech) mRNA in DNA-based therapeutic approaches in which pro-inflammatory signals from multiple sources must be suppressed simultaneously to achieve the desired therapeutic effect.

### Example 17: The Effect of the L Protein on saRNA Expression of a Reporter Protein

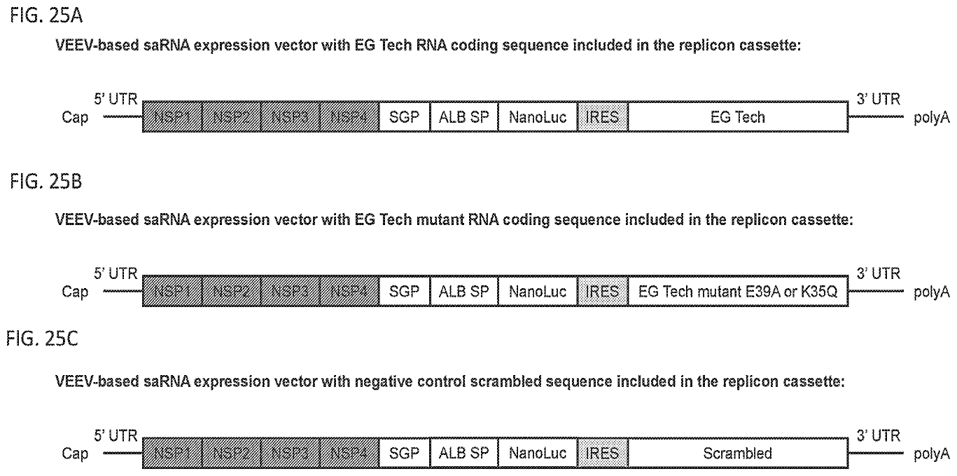

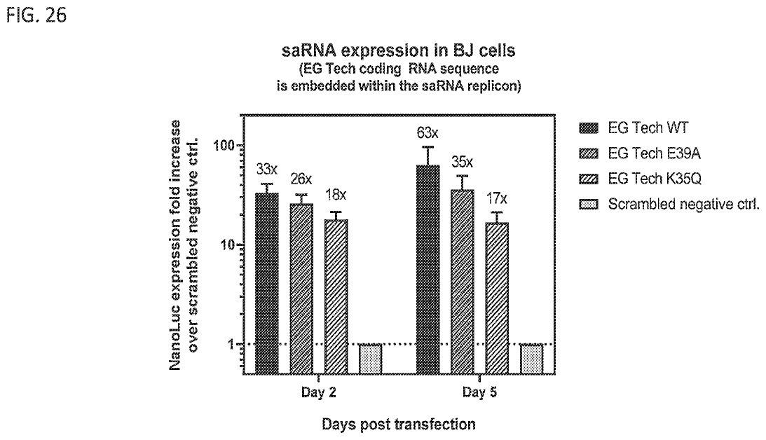

To evaluate the effect L protein (EG Tech) RNA to enhance GOI expression from saRNA vectors, constructs were designed in which the L protein (EG Tech)-coding, a L protein (EG Tech) mutant-coding RNA sequence, or a scrambled negative control sequences were included into the replicon cassette of the self-amplifying (saRNA) vector (FIGS. 25A-25C, respectively), in contrast to some of the earlier examples herein, in which the L protein (EG Tech) coding mRNA was co-transfected in trans alongside with the VEEV-based saRNA vector. The vector used in the current example was constructed by cloning the secreted Nanoluciferase (NanoLuc) followed by IRES-driven L protein (EG Tech) expression cassette into a commercially available VEEV-based saRNA vector (Simplicon SCR724, Millipore Sigma). L protein (EG Tech) WT encodes for the wild type protein sequence, L protein (EG Tech) mutants encodes for a version of the L protein (EG Tech) protein with a denoted amino acid mutation, and the scrambled negative control corresponds to the RNA sequence in which early stop codons are introduced to make sure a functional L protein (EG Tech) protein cannot be expressed. Different amino acid mutants of L protein (EG Tech) protein are used to modulate the activity of L protein (EG Tech) protein to modulate NCT.

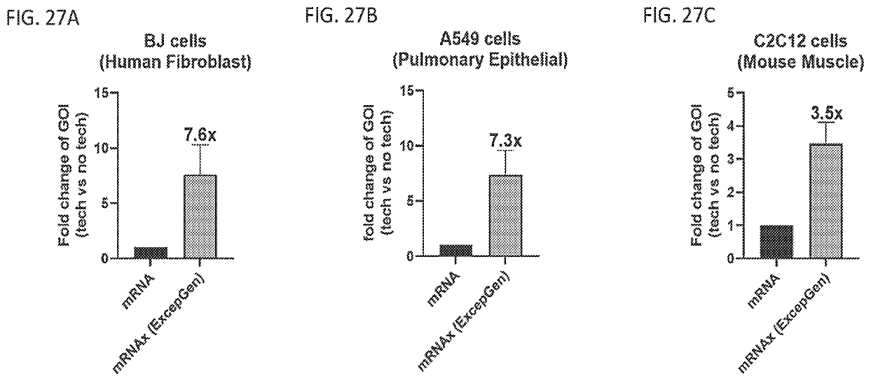

BJ cells were grown and transfected in with the designed saRNA construct as in earlier examples (Example 11) using 200 ng saRNA per well. Two days and five days after the transfection, Fluc expression in the transfected cells was measured using the Promega Nano-Glo Luciferase Assay System according to the manufacturer's instructions and normalized to the scrambled negative control sequence condition. The results are presented in FIGS. 27A-27C. It was observed that when L protein (EG Tech) coding RNA was included in the VEEV-based saRNA replicon, it increased NanoLuc expression from the saRNA 33×, 26× or 18× on Day 2 after transfection, depending on whether a WT, E39A (SEQ ID NO: 138) or K35Q L protein (SEQ ID NO: 148) (EG Tech) protein mutant was used, respectively. By Day 5 after transfection, the effects of L protein (EG Tech) in enhancing GOI expression grew stronger, increasing NanoLuc expression 63×, 35× or 17×, depending on whether a WT or a mutant version of L protein (EG Tech) was used, respectively.

In summary, these results show that the effect of L protein (EG Tech) RNA is universal and that it can be used to enhance GOI expression in different configurations: including using L protein (EG Tech) mRNA to enhance the expression of GOI mRNA when L protein (EG Tech) mRNA and GOI mRNA are co-transfected, when L protein (EG Tech) mRNA and GOI mRNA are part of a bicistronic mRNA expression vector, when L protein (EG Tech) mRNA is co-transfected with an saRNA expression vector, and when the L protein (EG Tech) RNA is an integral part of saRNA replicon.

### Example 18: The Effect of mRNA Expression of the L Protein on Natural Nucleoside Inflammatory Signals

To demonstrate further universality of L protein (EG Tech) mRNA to enhance GOI expression and to reduce the activation of pro-inflammatory signals, L protein (EG Tech) was tested in different cell lines side-by-side. The selected cell lines were BJ human fibroblasts, A549 human pulmonary epithelial cells and C2C12 mouse muscle cells. The experiment was performed as described in Example 2. Briefly, the cells were transfected with single mRNA constructs encoding both luciferase and the L protein (NCT inhibitor) protein separated by a p2A element. These mRNA constructs contained the ARCA cap and a 50%-50% mixture of natural uridines and pseudo-uridines. Due to the use of natural uridines in the mRNA, these mRNA constructs are inherently pro-inflammatory as they can activate innate immune signaling pathways in transfected cells, as known in the art. 48 hours after transfection, luciferase activity was measured using the Promega NanoGlo Luciferase Assay System. The activation of innate immune signaling pathways were measured using IFN-beta ELISA (R&D Biosystems) and the Biolegend LegendPlex Human Antivirus Panel according to manufacturer's instructions.

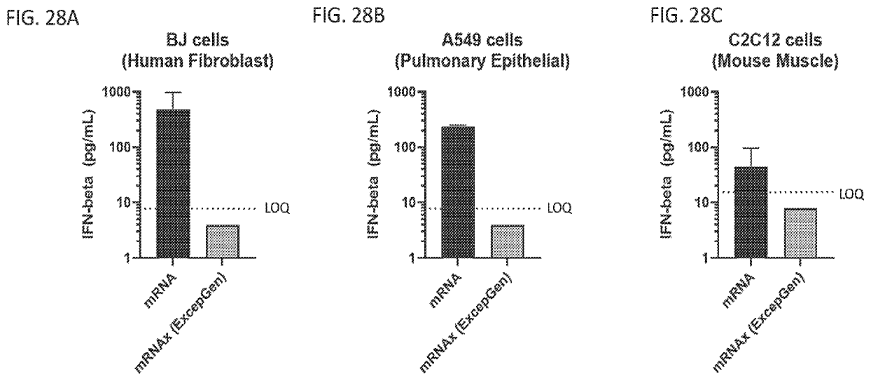

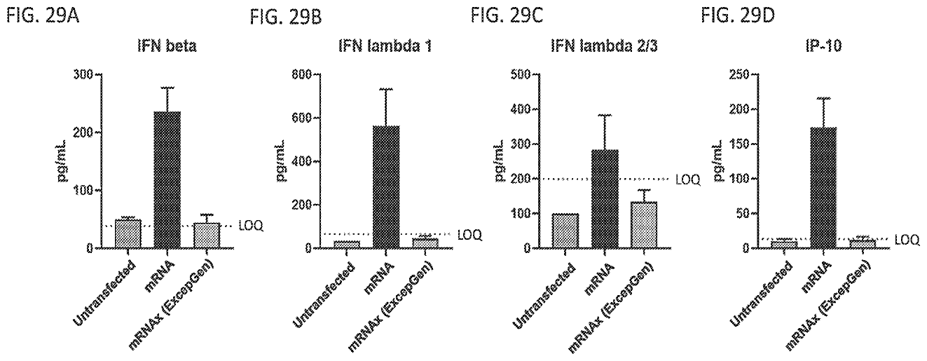

The results showed that the inclusion of L protein (EG Tech) RNA sequence in the mRNA expression vectors increased GOI expression 3.5×-7.6×, depending on the cell line used (FIGS. 27A-27C). Moreover, the use of L protein (EG Tech) virtually eliminated the induction of IFN-beta expression by the ARCA-capped and 50% natural uridine-containing mRNA in all tested cell lines, as measured by IFN-beta ELISA (FIGS. 28A-28C). Moreover, as assessed by the LegendPlex Human Antivirus Panel in BJ cells, the induction of several other proinflammatory modulators was eliminated as well (FIGS. 29A-29D).

These results demonstrate the applicability of L protein (EG Tech) to improve GOI expression while suppressing the activation of undesired inflammatory pathways across cell types.

1.-29. (canceled)

30. A method of reducing cell stress signals elicited by exposure to exogenous polynucleotides, the method comprising:
introducing a polynucleotide encoding a target protein into a cell; and
introducing a ribonucleic acid (RNA) encoding a nucleocytoplasmic transport (NCT) inhibitor protein into the cell, wherein expression of the NCT inhibitor protein reduces a cellular stress response associated with the introduction of the polynucleotide into the cell, thereby enhancing expression of the target protein.

31. The method of claim 30, wherein the cellular stress response comprises induction of inflammatory signaling.

32. The method of claim 30, wherein the cellular stress response comprises expression of interferon-beta.

33. The method of claim 30, wherein the polynucleotide comprises an RNA encoding the target protein.

34. The method of claim 33, wherein at least one of the RNA encoding the target protein or the RNA encoding the NCT inhibitor protein comprises at least one unmodified uridine nucleotide.

35. The method of claim 33, wherein at least one of the RNA encoding the target protein or the RNA encoding the NCT inhibitor protein is a messenger RNA (mRNA).

36. The method of claim 33, wherein at least one of the RNA encoding the target protein or the RNA encoding the NCT inhibitor protein is a self-amplifying RNA (saRNA).

37. The method of claim 33, wherein the RNA encoding the target protein and the RNA encoding the NCT inhibitor protein are part of the same RNA molecule.

38. The method of claim 33, wherein the RNA encoding the target protein and the RNA encoding the NCT inhibitor protein are part of separate RNA molecules.

39. The method of claim 30, wherein the polynucleotide comprises a deoxyribonucleic acid (DNA) encoding the target protein.

40. The method of claim 30, wherein the NCT inhibitor protein comprises a picornavirus leader (L) protein or a functional variant thereof.

41. The method of claim 40, wherein the picornavirus L protein is an Encephalomyocarditis virus (EMCV) L protein or a functional variant thereof.

42. The method of claim 30, wherein NCT inhibitor protein comprises an amino acid sequence having at least 70% identity to any one of SEQ ID NO: 65-SEQ ID NO: 69.

43. The method of claim 30, wherein the target protein comprises a vaccine antigen.

44. The method of claim 30, wherein at least one of the polynucleotide or the RNA is introduced into the cell via a delivery vehicle.

45. The method of claim 44, wherein the delivery vehicle comprises a lipid nanoparticle, a polymeric nanoparticle, a metal nanoparticle, or a viral vector.

46. The method of claim 30, wherein the RNA is introduced into the cell before or after the polynucleotide is introduced into the cell.

47. The method of claim 30, wherein the RNA is introduced into the cell together with the polynucleotide.

48. The method of claim 30, further comprising administering the polynucleotide and the RNA into a subject comprising the cell.

49. The method of claim 48, wherein the polynucleotide and the RNA are administered orally, subcutaneously, intravenously, intranasally, intradermally, transdermally, intraperitoneally, intramuscularly, intrapulmonary, vaginally, rectally, or intraocularly.
